(19) United States
(12) Patent Application Publication
(10) Pub. No.: US 2024/0148891 A1
(43) Pub. Date: May 9, 2024

(54) MUSCLE TARGETING COMPLEXES AND USES THEREOF FOR TREATING MYOTONIC DYSTROPHY

(71) Applicant: Dyne Therapeutics, Inc., Waltham, MA (US)

(72) Inventors: Romesh R. Subramanian, Framingham, MA (US); Mohammed T. QATANANI, Waltham, MA (US); Timothy WEEDEN, Waltham, MA (US); Cody A. DESJARDINS, Waltham, MA (US)

(73) Assignee: Dyne Therapeutics, Inc., Waltham, MA (US)

(21) Appl. No.: 17/769,467

(22) PCT Filed: Oct. 16, 2020
(86) PCT No.: PCT/US2020/055926

§ 371 Date: Apr. 15, 2022

Related U.S. Application Data
(60) Provisional application No. 62/923,421, filed on Oct. 18, 2019.

(51) Int. Cl. A61K 47/68; A61P 21/00

(52) U.S. Cl. CPC A61K 47/6849; A61P 21/00

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload inhibits expression or activity of a DMPK allele comprising a disease-associated-repeat. In some embodiments, the molecular payload is an oligonucleotide, such as an antisense oligonucleotide or RNAi oligonucleotide.

### RELATED APPLICATION

This application claims the benefit under 35 U.S.C. § 119(e) of U.S. Provisional Application Ser. No. 62/923,421, entitled “MUSCLE TARGETING COMPLEXES AND USES THEREOF FOR TREATING MYOTONIC DYSTROPHY,” filed on Oct. 18, 2019, the entire contents of which are incorporated herein by reference.

### FIELD OF THE INVENTION

The present application relates to targeting complexes for delivering molecular payloads (e.g., oligonucleotides) to cells and uses thereof, particularly uses relating to treatment of disease.

### REFERENCE TO THE SEQUENCE LISTING

The present application is being filed along with a Sequence Listing in electronic format. The Sequence Listing is provided as a file entitled D082470022WO00-SEQ.txt created on Oct. 16, 2020, which is 154 kilobytes in size. The information in electronic format of the sequence listing is incorporated herein by reference in its entirety.

### BACKGROUND

Myotonic dystrophy (DM) is a dominantly inherited genetic disease that is characterized by myotonia, muscle loss or degeneration, diminished muscle function, insulin resistance, cardiac arrhythmia, smooth muscle dysfunction, and neurological abnormalities. DM is the most common form of adult-onset muscular dystrophy, with a worldwide incidence of about 1 in 8000 people worldwide. Two types of the disease, myotonic dystrophy type 1 (DM1) and myotonic dystrophy type 2 (DM2), have been described. DM1, the more common form of the disease, results from a repeat expansion of a CTG trinucleotide repeat in the 3′ non-coding region of DMPK on chromosome 19; DM2 results from a repeat expansion of a CCTG tetranucleotide repeat in the first intron of ZNF9 on chromosome 3. In DM1 patients, the repeat expansion of a CTG trinucleotide repeat, which may comprise greater than ˜50 to ˜3,000+ total repeats, leads to generation of toxic RNA repeats capable of forming hairpin structures that bind essential intracellular proteins, e.g. muscleblind-like proteins, with high affinity resulting in protein sequestration and the loss-of-function phenotypes that are characteristic of the disease. Apart from supportive care and treatments to address the symptoms of the disease, no effective therapeutic for DM1 is currently available.

### SUMMARY

According to some aspects, the present disclosure provide methods of treating a subject having an expansion of a disease-associated-repeat of a DMPK allele that is associated with myotonic dystrophy, the method comprising administering a muscle-targeting complex to the subject, wherein the complex comprises a muscle-targeting agent covalently linked to a molecular payload configured for inhibiting expression or activity of a DMPK allele, and wherein the administration results in inhibition of DMPK expression in muscle tissues by at least 30% (e.g., 40-60%), relative to a control, for a duration of time in the range of 4-8, 5-10, 8-12, 10-14, or 8-16 weeks following administration of the complex.

Also provided are methods of inhibiting DMPK expression in a subject, the method comprising administering a muscle-targeting complex to the subject, wherein the complex comprises a muscle-targeting agent covalently linked to a molecular payload configured for inhibiting expression of a DMPK allele, and wherein the administration results in inhibition of DMPK expression in muscle tissues by at least 30% (e.g., 40-60%), relative to a control, for a duration of time in the range of 4-8, 5-10, 8-12, 10-14, or 8-16 weeks following administration of the complex.

In some embodiments, the complex is administered at a molecular payload dose of 1-15 mg/kg. In some embodiments, the complex is administered at a molecular payload dose of 10 mg/kg. In some embodiments, the complex is administered to the subject in a single dose.

In some embodiments, the muscle-targeting agent is a muscle-targeting antibody. In some embodiments, the muscle-targeting antibody is a transferrin receptor antibody. In some embodiments, the muscle-targeting antibody binds to an epitope of a sequence in the range of C89 to F760 of SEQ ID NO: 1-3.

In some embodiments, the molecular payload is an oligonucleotide. In some embodiments, the oligonucleotide is a single stranded antisense oligonucleotide. In some embodiments, the oligonucleotide is a gapmer.

In some embodiments, the oligonucleotide comprises at least 15 consecutive nucleotides of a sequence comprising any one of SEQ ID NOs: 45-280. In some embodiments, the oligonucleotide comprises a sequence comprising any one of SEQ ID NOs: 45-280, optionally wherein the oligonucleotide comprises a sequence comprising any one of SEQ ID NO: 56, 59, 69, 71, 77, 79, 85, 87, 92, 93, 98, 100, 109, 112, 115, 119, 145, or 161. In some embodiments, the oligonucleotide comprises a region of complementarity to at least 15 consecutive nucleotides of any one of SEQ ID NO: 281-516.

In some embodiments, the oligonucleotide comprises one or more modified nucleotides. In some embodiments, the one or more modified nucleotides is selected from the group consisting of: 5′-methyl-cytosine, 2′-O-methyl, 2′-fluoro (2′-F), 2′-O-methoxyethyl (2′-MOE), 2′,4′-constrained 2′-O-ethyl (cEt), locked nucleic acid (LNA) nucleotides. In some embodiments, the oligonucleotide comprises at least one modified internucleotide linkage. In some embodiments, the at least one modified internucleotide linkage is a phosphorothioate linkage. In some embodiments, the phosphorothioate linkage is in the Rp stereochemical conformation and/or in the Sp stereochemical conformation.

In some embodiments, the muscle-targeting agent is covalently linked to the molecular payload via a linker. In some embodiments, the linker is a cleavable linker. In some embodiments, linker comprises a valine-citrulline dipeptide sequence.

In some embodiments, the subject is a human subject having myotonic dystrophy, type 1 (DM1).

Other aspects of the present disclosure provide oligonucleotides (e.g., single stranded antisense oligonucleotides such as gapmers) complementary to a non-repeat region in a DMPK allele comprising the disease-associated-repeat expansion, wherein the oligonucleotide comprises a region of complementarity to at least 15 consecutive nucleotides of any one of SEQ ID NO: 281-516, one or more modified nucleotides selected from the group consisting of: 5′-methyl-cytosine, 2′-O-methyl, 2′-fluoro, 2′-O-methoxyethyl (2′-MOE), 2′,4′-constrained 2′-O-ethyl (cEt), locked nucleic acid (LNA) nucleotides, and one or more phosphorothioate internucleotide linkages, optionally wherein the oligonucleotide is a gapmer.

Further provided herein are complexes comprising an anti-transferrin receptor antibody covalently linked to the oligonucleotides described herein via a cleavable linker, In some embodiments, the transferrin receptor antibody binds to an epitope of a sequence in the range of C89 to F760 of SEQ ID NO: 1-3. In some embodiments, the cleavable linker comprises a valine-citrulline dipeptide sequence.

In some aspects, methods of treating myotonic dystrophy, type 1 (DM1) are provided. In some embodiments, the method comprising administering to a subject in need thereof an effective amount of the complex described herein.

In some embodiments, the administration results in inhibition of DMPK expression in muscle tissues by 40-60% in the subject, relative to a control, for a duration of time in the range of 4-8, 5-10, 8-12, 10-14, or 8-16 weeks following administration of the complex. In some embodiments, the complex is administered at a molecular payload dose of 1-15 mg/kg. In some embodiments, the complex is administered at a molecular payload dose of 10 mg/kg.

### BRIEF DESCRIPTION OF THE DRAWINGS

FIG. 1 depicts a non-limiting schematic showing the effect of transfecting Hepa 1-6 cells with an antisense oligonucleotide that targets DMPK (DTX-P-060) on expression levels of DMPK relative to a vehicle transfection;

FIG. 2A depicts a non-limiting schematic showing an HIL-HPLC trace obtained during purification of a muscle targeting complex comprising an anti-transferrin receptor antibody covalently linked to a DMPK antisense oligonucleotide.

FIG. 2B depicts a non-limiting image of an SDS-PAGE analysis of a muscle targeting complex.

FIG. 3 depicts a non-limiting schematic showing the ability of a muscle targeting complex (DTX-C-008) comprising DTX-P-060 to reduce expression levels of DMPK.

FIGS. 4A-4E depict non-limiting schematics showing the ability of a muscle targeting complex (DTX-C-008) comprising DTX-P-060 to reduce expression levels of DMPK in mouse muscle tissues in vivo, relative to a vehicle experiment. (N=3 C57B1/6 WT mice)

FIGS. 5A-5B depict non-limiting schematics showing the tissue selectivity of a muscle targeting complex (DTX-C-008) comprising DTX-P-060. The muscle targeting complex (DTX-C-008) comprising DTX-P-060 does not reduce expression levels of DMPK in mouse brain or spleen tissues in vivo, relative to a vehicle experiment. (N=3 C57B1/6 WT mice)

FIGS. 6A-6F depict non-limiting schematics showing the ability of a muscle targeting complex (DTX-C-008) comprising DTX-P-060 to reduce expression levels of DMPK in mouse muscle tissues in vivo, relative to a vehicle experiment. (N=5 C57B1/6 WT mice)

FIGS. 7A-7L depict non-limiting schematics showing the ability of a muscle targeting complex (DTX-C-012) comprising DTX-P-060 to reduce expression levels of DMPK in cynomolgus monkey muscle tissues in vivo, relative to a vehicle experiment and compared to a naked DMPK ASO (DTX-P-060). (N=3 male cynomolgus monkeys)

FIGS. 8A-8B depict non-limiting schematics showing the ability of a muscle targeting complex (DTX-C-012) comprising DTX-P-060 to reduce expression levels of DMPK in cynomolgus monkey smooth muscle tissues in vivo, relative to a vehicle experiment and compared to a naked DMPK ASO (DTX-P-060). (N=3 male cynomolgus monkeys)

FIGS. 9A-9D depict non-limiting schematics showing the tissue selectivity of a muscle targeting complex (DTX-C-012) comprising DTX-P-060. The muscle targeting complex comprising DMPK-ASO does not reduce expression levels of DMPK in cynomolgus monkey liver, kidney, brain, or spleen tissues in vivo, relative to a vehicle experiment. (N=3 male cynomolgus monkeys)

FIG. 10 shows normalized DMPK mRNA tissue expression levels across several tissue types in cynomolgus monkeys. (N=3 male cynomolgus monkeys) FIGS. 11A-11B depict non-limiting schematics showing the ability of a muscle

targeting complex (DTX-C-008) comprising DTX-P-060 to reduce expression levels of DMPK in mouse muscle tissues in vivo for up to 28 days after dosing with DTX-C-008, relative to a vehicle experiment and compared to a naked DMPK ASO (DTX-P-060).

FIG. 12 shows that a single dose of a muscle targeting complex (DTX-C-012) comprising DTX-P-060 is safe and tolerated in cynomolgus monkeys. (N=3 male cynomolgus monkeys)

FIGS. 13A-13B depict non-limiting schematics showing the ability of a muscle targeting complex (DTX-C-008) comprising DTX-P-060 to reduce expression levels of DMPK in mouse muscle tissues in vivo for up to twelve weeks after dosing with DTX-C-008, relative to a vehicle experiment; and compared to a control complex (DTX-C-007) and naked DMPK ASO (DTX-P-060). (N=5 C57B1/6 WT mice)

### DETAILED DESCRIPTION OF INVENTION

Aspects of the disclosure relate to a recognition that while certain molecular payloads (e.g., oligonucleotides, peptides, small molecules) can have beneficial effects in muscle cells, it has proven challenging to effectively target such cells. As described herein, the present disclosure provides complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having a rare muscle disease. For example, in some embodiments, complexes are provided for targeting a DMPK allele that comprises an expanded disease-associated-repeat to treat subjects having DM1. In some embodiments, complexes provided herein may comprise oligonucleotides that inhibit expression of a DMPK allele comprising an expanded disease-associated-repeat. As another example, complexes may comprise oligonucleotides that interfere with the binding of a disease-associated DMPK mRNA to a muscleblind-like protein (e.g., MBNL1, 2, and/or 3), thereby reducing a toxic effect of a disease-associated DMPK allele. In some embodiments, synthetic nucleic acid payloads (e.g., DNA or RNA payloads) may be used that express one or more proteins that reduce a toxic effect of a disease-associated DMPK allele. In some embodiments, complexes may comprise molecular payloads of synthetic cDNAs and/or synthetic mRNAs, e.g., that express one or more muscleblind-like-proteins (e.g., MBNL1, 2, and/or 3) or fragments thereof. In some embodiments, complexes may comprise molecular payloads such as guide molecules (e.g., guide RNAs) that are capable of targeting nucleic acid programmable nucleases (e.g., Cas9) to a sequence at or near a disease-associated repeat sequence of DMPK. In some embodiments, such nucleic programmable nucleases could be used to cleave part or all of a disease-associated repeat sequence from a DMPK gene.

Further aspects of the disclosure, including a description of defined terms, are provided below.

### I. Definitions

Administering: As used herein, the terms “administering” or “administration” means to provide a complex to a subject in a manner that is physiologically and/or pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond.

In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferring receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Disease-associated-repeat: As used herein, the term “disease-associated-repeat” refers to a repeated nucleotide sequence at a genomic location for which the number of units of the repeated nucleotide sequence is correlated with and/or directly or indirectly contributes to, or causes, genetic disease. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, embodiments, the disease-associated-repeat comprises CAG repeats, CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease.

DMPK: As used herein, the term “DMPK” refers to a gene that encodes myotonin-protein kinase (also known as myotonic dystrophy protein kinase or dystrophia myotonica protein kinase), a serine/threonine protein kinase. Substrates for this enzyme may include myogenin, the beta-subunit of the L-type calcium channels, and phospholemman. In some embodiments, DMPK may be a human (Gene ID: 1760), non-human primate (e.g., Gene ID: 456139, Gene ID: 715328), or rodent gene (e.g., Gene ID: 13400). In humans, a CTG repeat expansion in the 3′ non-coding, untranslated region of DMPK is associated with myotonic dystrophy type I (DM1). In addition, multiple human transcript variants (e.g., as annotated under GenBank RefSeq Accession Numbers: NM 001081563.2, NM 004409.4, NM_001081560.2, NM_001081562.2, NM_001288764.1, NM_001288765.1, and NM_001288766.1) have been characterized that encode different protein isoforms.

DMPK allele: As used herein, the term “DMPK allele” refers to any one of alternative forms (e.g., wild-type or mutant forms) of a DMPK gene. In some embodiments, a DMPK allele may encode for wild-type myotonin-protein kinase that retains its normal and typical functions. In some embodiments, a DMPK allele may comprise one or more disease-associated-repeat expansions. In some embodiments, normal subjects have two DMPK alleles comprising in the range of 5 to 37 repeat units. In some embodiments, the number of CTG repeat units in subjects having DM1 is in the range of ˜50 to −3,000+ with higher numbers of repeats leading to an increased severity of disease. In some embodiments, mildly affected DM1 subjects have at least one DMPK allele having in the range of 50 to 150 repeat units. In some embodiments, subjects with classic DM1 have at least one DMPK allele having in the range of 100 to 1,000 or more repeat units. In some embodiments, subjects having DM1 with congenital onset may have at least one DMPK allele comprising more than 2,000 repeat units.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Internucleotide linkage: An “internucleotide linkage,” as used herein, refers to the linkage between two nucleosides/nucleotides in an oligonucleotide. An internucleotide linkage may be a natural internucleotide linkage (e.g., a phosphodiester bond) or a modified internucleotide linkage (e.g., a phosphorothioate linkage).

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Myotonic dystrophy (DM): As used herein, the term “Myotonic dystrophy (DM)” refers to a genetic disease caused by mutations in the DMPK gene or CNBP (ZNF9) gene that is characterized by muscle loss, muscle weakening, and muscle function. Two types of the disease, myotonic dystrophy type 1 (DM1) and myotonic dystrophy type 2 (DM2), have been described. DM1 is associated with an expansion of a CTG trinucleotide repeat in the 3′ non-coding region of DMPK. DM2 is associated with an expansion of a CCTG tetranucleotide repeat in the first intron of ZNF9. In both DM1 and DM2, the nucleotide expansions lead to toxic RNA repeats capable of forming hairpin structures that bind critical intracellular proteins, e.g., muscleblind-like proteins, with high affinity. Myotonic dystrophy, the genetic basis for the disease, and related symptoms are described in the art (see, e.g. Thornton, C. A., “Myotonic Dystrophy” Neurol Clin. (2014), 32(3): 705-719.; and Konieczny et al. “Myotonic dystrophy: candidate small molecule therapeutics” Drug Discovery Today (2017), 22:11.) In some embodiments, subjects are born with a variation of DM1 called congenital myotonic dystrophy. Symptoms of congenital myotonic dystrophy are present from birth and include weakness of all muscles, breathing problems, clubfeet, developmental delays and intellectual disabilities. DM1 is associated with Online Mendelian Inheritance in Man (OMIM) Entry #160900. DM2 is associated with OMIM Entry #602668.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise all phosphodiester internucleotide linkages. In some embodiments, an oligonucleotide may comprise one or more modified internucleotide linkage. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of a oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a disease-associated-repeat expansion, e.g., in a DMPK allele.

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

### II. Complexes

Provided herein are complexes that comprise a targeting agent, e.g. an antibody, covalently linked to a molecular payload. In some embodiments, a complex comprises a muscle-targeting antibody covalently linked to a oligonucleotide. A complex may comprise an antibody that specifically binds a single antigenic site or that binds to at least two antigenic sites that may exist on the same or different antigens.

A complex may be used to modulate the activity or function of at least one gene, protein, and/or nucleic acid. In some embodiments, the molecular payload present with a complex is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a disease-associated repeat in muscle cells.

In some embodiments, a complex comprises a muscle-targeting agent, e.g. an anti-transferrin receptor antibody, covalently linked to a molecular payload, e.g. an antisense oligonucleotide that targets a disease-associated repeat, e.g. DMPK allele.

A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure. For example, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-transferrin receptor antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin Ilb” Mol Immunol. 2003 March, 39(13):78309; the entire contents of each of which are incorporated herein by reference.

a. Anti-Transferrin Receptor Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to as an anti-transferrin receptor antibody. Antibodies that bind, e.g. specifically bind, to a transferrin receptor may be internalized into the cell, e.g. through receptor-mediated endocytosis, upon binding to a transferrin receptor.

It should be appreciated that anti-transferrin receptor antibodies may be produced, synthesized, and/or derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-transferrin antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

Any appropriate anti-transferrin receptor antibodies may be used in the complexes disclosed herein. Examples of anti-transferrin receptor antibodies, including associated references and binding epitopes are listed in Table 1. In some embodiments, the anti-transferrin receptor antibody comprises the complementarity determining regions (CDR-H1, CDR-H2, CDR-H3, CDR-L1, CDR-L2, and CDR-L3) of any of the anti-transferrin receptor antibodies provided herein, e.g., anti-transferrin receptor antibodies listed in Table 1.

TABLE 1
List of anti-transferrin receptor antibody clones, including
associated references and binding epitope information.

| Antibody | | |
|---|---|---|
| Clone Name | Reference(s) | Epitope/Notes |
| OKT9 | U.S. Pat. No. 4,364,934, filed Dec. 4, | Apical domain of TfR |
| | 1979, entitled “MONOCLONAL | (residues 305-366 of |
| | ANTIBODY TO A HUMAN EARLY | human TfR sequence |
| | THYMOCYTE ANTIGEN AND | XM_052730.3, |
| | METHODS FOR PREPARING SAME” | available in GenBank) |
| | Schneider C. et al. “Structural features of | |
| | the cell surface receptor for transferrin that | |
| | is recognized by the monoclonal antibody | |
| | OKT9.” J Biol Chem. 1982, 257: 14, | |
| | 8516-8522. | |
| (From JCR) | WO 2015/098989, filed Dec. 24, 2014, | Apical domain |
| Clone M11 | “Novel anti-Transferrin receptor antibody | (residues 230-244 and |
| Clone M23 | that passes through blood-brain barrier” | 326-347 of TfR) and |
| Clone M27 | U.S. Pat. No. 9,994,641, filed Dec. 24, | protease-like domain |
| Clone B84 | 2014, “Novel anti-Transferrin receptor | (residues 461-473) |
| | antibody that passes through blood-brain | |
| | barrier” | |
| (From Genentech) | WO 2016/081643, filed May 26, 2016, | Apical domain and |
| 7A4, 8A2, 15D2, | entitled “ANTI-TRANSFERRIN | non-apical regions |
| 10D11, 7B10, | RECEPTOR ANTIBODIES AND | |
| 15G11, 16G5, | METHODS OF USE” | |
| 13C3, 16G4, | U.S. Pat. No. 9,708,406, filed | |
| 16F6, 7G7, 4C2, | May 20, 2014, “Anti-transferrin receptor | |
| 1B12, and 13D4 | antibodies and methods of use” | |
| (From Armagen) | Lee et al. “Targeting Rat Anti-Mouse | |
| 8D3 | Transferrin Receptor Monoclonal | |
| | Antibodies through Blood-Brain Barrier in | |
| | Mouse” 2000, J Pharmacol. Exp. Ther., | |
| | 292: 1048-1052. | |
| | U.S. Patent Application 2010/077498, filed | |
| | Sep. 11, 2008, entitled “COMPOSITIONS | |
| | AND METHODS FOR BLOOD-BRAIN | |
| | BARRIER DELIVERY IN THE MOUSE” | |
| OX26 | Haobam, B. et al. 2014. Rab17-mediated | |
| | recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of the | |
| | human transferrin receptor in plant | |
| | cells: effects of tyrphostin A23 and | |
| | brefeldin A. Plant J 48: 757-70 (2006). | |
| 1A1B2, 66IG10, | Commercially available anti-transferrin | Novus Biologicals |
| MEM-189, | receptor antibodies. | 8100 Southpark Way, |
| JF0956, | | A-8 Littleton CO |
| 29806, 1A1B2, | | 80120 |
| TFRC/1818, | | |
| 1E6, 66Ig10, | | |
| TFRC/1059, Q1/71, | | |
| 23D10, 13E4, | | |
| TFRC/1149, | | |
| ER-MP21, | | |
| YTA74.4, BU54, | | |
| 2B6, RI7 217 | | |
| (From INSERM) | U.S. Patent Application 2011/0311544A1, | Does not compete |
| BA120g | filed Jun. 15, 2005, entitled “ANTI-CD71 | with OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti-transferrin | BioXcell |
| 5E9C11, OKT9 | receptor antibodies. | 10 Technology Dr., |
| (BE0023 clone) | | Suite 2B |
| | | West Lebanon, NH |
| | | 03784-1671 USA |
| BK19.9, B3/25, | Gatter, K. C. et al. “Transferrin | |
| T56/14 and T58/1 | receptors in human tissues: their | |
| | distribution and possible clinical | |
| | relevance.” J Clin Pathol. 1983 | |
| | May; 36(5): 539-45. | |

In some embodiments, the muscle-targeting agent is an anti-transferrin receptor antibody. In some embodiment, an anti-transferrin receptor antibody specifically binds to a transferrin protein having an amino acid sequence as disclosed herein. In some embodiments, an anti-transferrin receptor antibody may specifically bind to any extracellular epitope of a transferrin receptor or an epitope that becomes exposed to an antibody, including the apical domain, the transferrin binding domain, and the protease-like domain. In some embodiments, an anti-transferrin receptor antibody binds to an amino acid segment of a human or non-human primate transferrin receptor, as provided in SEQ ID Nos. 1-3 in the range of amino acids C89 to F760. In some embodiments, an anti-transferrin receptor antibody specifically binds with binding affinity of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. Anti-transferrin receptor antibodies used herein may be capable of competing for binding with other anti-transferrin receptor antibodies, e.g. OKT9, 8D3, that bind to transferrin receptor with 10⁻³ M, 10⁻⁴ M, 10⁻⁵ M, 10⁻⁶ M, 10⁻⁷ M, or less.

An example human transferrin receptor amino acid sequence, corresponding to NCBI sequence NP_003225.2 (transferrin receptor protein 1 isoform 1, Homo sapiens) is as follows:

(SEQ ID NO: 1)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMESDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLOWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

An example non-human primate transferrin receptor amino acid sequence, corresponding to NCBI sequence NP_001244232.1 (transferrin receptor protein 1, Macaca mulatta) is as follows:

(SEQ ID NO: 2)
MMDQARSAFSNLFGGEPLSYTRFSLARQVDGDNSHVEMKLGVDEEENTD
NNTKPNGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGQFVIKLTHDTELNLDYERYNSQLLLFLRDLNQYRADVKEMGL
SLOWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 3)
MMDQARSAFSNLFGGEPLSYTRFSLARQVDGDNSHVEMKLGVDEEENTD
NNTKANGTKPKRCGGNICYGTIAVIIFFLIGFMIGYLGYCKGVEPKTEC
ERLAGTESPAREEPEEDFPAAPRLYWDDLKRKLSEKLDTTDFTSTIKLL
NENLYVPREAGSQKDENLALYIENQFREFKLSKVWRDQHFVKIQVKDSA
QNSVIIVDKNGGLVYLVENPGGYVAYSKAATVTGKLVHANFGTKKDFED
LDSPVNGSIVIVRAGKITFAEKVANAESLNAIGVLIYMDQTKFPIVKAD
LSFFGHAHLGTGDPYTPGFPSFNHTQFPPSQSSGLPNIPVQTISRAAAE
KLFGNMEGDCPSDWKTDSTCKMVTSENKSVKLTVSNVLKETKILNIFGV
IKGFVEPDHYVVVGAQRDAWGPGAAKSSVGTALLLKLAQMFSDMVLKDG
FQPSRSIIFASWSAGDFGSVGATEWLEGYLSSLHLKAFTYINLDKAVLG
TSNFKVSASPLLYTLIEKTMQDVKHPVTGRSLYQDSNWASKVEKLTLDN
AAFPFLAYSGIPAVSFCFCEDTDYPYLGTTMDTYKELVERIPELNKVAR
AAAEVAGOFVIKLTHDTELNLDYERYNSQLLLFLRDLNQYRADVKEMGL
SLOWLYSARGDFFRATSRLTTDFRNAEKRDKFVMKKLNDRVMRVEYYFL
SPYVSPKESPFRHVFWGSGSHTLSALLESLKLRRQNNSAFNETLFRNQL
ALATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 4)
MMDQARSAFSNLFGGEPLSYTRFSLARQVDGDNSHVEMKLAADEEENAD
NNMKASVRKPKRFNGRLCFAAIALVIFFLIGFMSGYLGYCKRVEQKEEC
VKLAETEETDKSETMETEDVPTSSRLYWADLKTLLSEKLNSIEFADTIK
QLSQNTYTPREAGSQKDESLAYYIENQFHEFKFSKVWRDEHYVKIQVKS
SIGQNMVTIVQSNGNLDPVESPEGYVAFSKPTEVSGKLVHANFGTKKDF
EELSYSVNGSLVIVRAGEITFAEKVANAQSFNAIGVLIYMDKNKFPVVE
ADLALFGHAHLGTGDPYTPGFPSFNHTQFPPSQSSGLPNIPVQTISRAA
AEKLFGKMEGSCPARWNIDSSCKLELSQNQNVKLIVKNVLKERRILNIF
GVIKGYEEPDRYVVVGAQRDALGAGVAAKSSVGTGLLLKLAQVFSDMIS
KDGFRPSRSIIFASWTAGDFGAVGATEWLEGYLSSLHLKAFTYINLDKV
VLGTSNFKVSASPLLYTLMGKIMQDVKHPVDGKSLYRDSNWISKVEKLS
FDNAAYPFLAYSGIPAVSFCFCEDADYPYLGTRLDTYEALTQKVPQLNQ
MVRTAAEVAGQLIIKLTHDVELNLDYEMYNSKLLSFMKDLNQFKTDIRD
MGLSLOWLYSARGDYFRATSRLTTDFHNAEKTNRFVMREINDRIMKVEY
HFLSPYVSPRESPFRHIFWGSGSHTLSALVENLKLRQKNITAFNETLFR
NQLALATWTIQGVANALSGDIWNIDNEF

In some embodiments, an anti-transferrin receptor antibody binds to an amino acid segment of the receptor as follows:

(SEQ ID NO: 5)
FVKIQVKDSAQNSVIIVDKNGRLVYLVENPGGYVAYSKAATVTGKLVHA
NFGTKKDFEDLYTPVNGSIVIVRAGKITFAEKVANAESLNAIGVLIYMD
QTKFPIVNAELSFFGHAHLGTGDPYTPGFPSFNHTQFPPSRSSGLPNIP
VQTISRAAAEKLFGNMEGDCPSDWKTDSTCRMVTSESKNVKLTVSNVLK
E

and does not inhibit the binding interactions between transferrin receptors and transferrin and/or human hemochromatosis protein (also known as HFE).

Appropriate methodologies may be used to obtain and/or produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

Some aspects of the disclosure provide proteins that bind to transferrin receptor (e.g., an extracellular portion of the transferrin receptor). In some embodiments, transferrin receptor antibodies provided herein bind specifically to transferrin receptor (e.g., human transferrin receptor). Transferrin receptors are internalizing cell surface receptors that transport transferrin across the cellular membrane and participate in the regulation and homeostasis of intracellular iron levels. In some embodiments, transferrin receptor antibodies provided herein bind specifically to transferrin receptor from human, non-human primates, mouse, rat, etc. In some embodiments, transferrin receptor antibodies provided herein bind to human transferrin receptor. In some embodiments, transferrin receptor antibodies provided herein specifically bind to human transferrin receptor. In some embodiments, transferrin receptor antibodies provided herein bind to an apical domain of human transferrin receptor. In some embodiments, transferrin receptor antibodies provided herein specifically bind to an apical domain of human transferrin receptor.

In some embodiments, transferrin receptor antibodies of the present disclosure include one or more of the CDR-H (e.g., CDR-H1, CDR-H2, and CDR-H3) amino acid sequences from any one of the anti-transferrin receptor antibodies selected from Table 1. In some embodiments, transferrin receptor antibodies include the CDR-H1, CDR-H2, and CDR-H3 as provided for any one of the anti-transferrin receptor antibodies selected from Table 1. In some embodiments, anti-transferrin receptor antibodies include the CDR-L1, CDR-L2, and CDR-L3 as provided for any one of the anti-transferrin receptor antibodies selected from Table 1. In some embodiments, anti-transferrin antibodies include the CDR-H1, CDR-H2, CDR-H3, CDR-L1, CDR-L2, and CDR-L3 as provided for any one of the anti-transferrin receptor antibodies selected from Table 1. The disclosure also includes any nucleic acid sequence that encodes a molecule comprising a CDR-H1, CDR-H2, CDR-H3, CDR-L1, CDR-L2, or CDR-L3 as provided for any one of the anti-transferrin receptor antibodies selected from Table 1. In some embodiments, antibody heavy and light chain CDR3 domains may play a particularly important role in the binding specificity/affinity of an antibody for an antigen. Accordingly, anti-transferrin receptor antibodies of the disclosure may include at least the heavy and/or light chain CDR3s of any one of the anti-transferrin receptor antibodies selected from Table 1.

In some examples, any of the anti-transferrin receptor antibodies of the disclosure have one or more CDR (e.g., CDR-H or CDR-L) sequences substantially similar to any of the CDR-H1, CDR-H2, CDR-H3, CDR-L1, CDR-L2, and/or CDR-L3 sequences from one of the anti-transferrin receptor antibodies selected from Table 1. In some embodiments, the position of one or more CDRs along the VH (e.g., CDR-H1, CDR-H2, or CDR-H3) and/or VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or CDR-H3 described herein may be one, two, three, four, five or more amino acids shorter than one or more of the CDRs described herein (e.g., CDRS from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or CDR-H3 described herein may be one, two, three, four, five or more amino acids longer than one or more of the CDRs described herein (e.g., CDRS from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 1) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRS from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRS from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-transferrin receptor antibodies of the disclosure have one or more CDR (e.g., CDR-H or CDR-L) sequences substantially similar to any one of the anti-transferrin receptor antibodies selected from Table 1. For example, the antibodies may include one or more CDR sequence(s) from any of the anti-transferrin receptor antibodies selected from Table 1 containing up to 5, 4, 3, 2, or 1 amino acid residue variations as compared to the corresponding CDR region in any one of the CDRs provided herein (e.g., CDRs from any of the anti-transferrin receptor antibodies selected from Table 1) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide transferrin receptor antibodies that comprise one or more of the heavy chain variable (VH) and/or light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-transferrin receptor antibodies selected from Table 1. In some embodiments, any of the VL domains provided herein include one or more of the CDR-L sequences (e.g., CDR-L1, CDR-L2, and CDR-L3) provided herein, for example, any of the CDR-L sequences provided in any one of the anti-transferrin receptor antibodies selected from Table 1.

In some embodiments, anti-transferrin receptor antibodies of the disclosure include any antibody that includes a heavy chain variable domain and/or a light chain variable domain of any anti-transferrin receptor antibody, such as any one of the anti-transferrin receptor antibodies selected from Table 1. In some embodiments, anti-transferrin receptor antibodies of the disclosure include any antibody that includes the heavy chain variable and light chain variable pairs of any anti-transferrin receptor antibody, such as any one of the anti-transferrin receptor antibodies selected from Table 1.

Aspects of the disclosure provide anti-transferrin receptor antibodies having a heavy chain variable (VH) and/or a light chain variable (VL) domain amino acid sequence homologous to any of those described herein. In some embodiments, the anti-transferrin receptor antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-transferrin receptor antibody, such as any one of the anti-transferrin receptor antibodies selected from Table 1. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-transferrin receptor antibodies provided herein comprise a heavy chain variable sequence and a light chain variable sequence that comprises a framework sequence that is at least 75%, 80%, 85%, 90%, 95%, 98%, or 99% identical to the framework sequence of any anti-transferrin receptor antibody, such as any one of the anti-transferrin receptor antibodies selected from Table 1.

In some embodiments, an anti-transferrin receptor antibody, which specifically binds to transferrin receptor (e.g., human transferrin receptor), comprises a light chain variable VL domain comprising any of the CDR-L domains (CDR-L1, CDR-L2, and CDR-L3), or CDR-L domain variants provided herein, of any of the anti-transferrin receptor antibodies selected from Table 1. In some embodiments, an anti-transferrin receptor antibody, which specifically binds to transferrin receptor (e.g., human transferrin receptor), comprises a light chain variable VL domain comprising the CDR-L1, the CDR-L2, and the CDR-L3 of any anti-transferrin receptor antibody, such as any one of the anti-transferrin receptor antibodies selected from Table 1. In some embodiments, the anti-transferrin receptor antibody comprises a light chain variable (VL) region sequence comprising one, two, three or four of the framework regions of the light chain variable region sequence of any anti-transferrin receptor antibody, such as any one of the anti-transferrin receptor antibodies selected from Table 1. In some embodiments, the anti-transferrin receptor antibody comprises one, two, three or four of the framework regions of a light chain variable region sequence which is at least 75%, 80%, 85%, 90%, 95%, or 100% identical to one, two, three or four of the framework regions of the light chain variable region sequence of any anti-transferrin receptor antibody, such as any one of the anti-transferrin receptor antibodies selected from Table 1. In some embodiments, the light chain variable framework region that is derived from said amino acid sequence consists of said amino acid sequence but for the presence of up to 10 amino acid substitutions, deletions, and/or insertions, preferably up to 10 amino acid substitutions. In some embodiments, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region.

In some embodiments, an anti-transferrin receptor antibody that specifically binds to transferrin receptor comprises the CDR-L1, the CDR-L2, and the CDR-L3 of any anti-transferrin receptor antibody, such as any one of the anti-transferrin receptor antibodies selected from Table 1. In some embodiments, the antibody further comprises one, two, three or all four VL framework regions derived from the VL of a human or primate antibody. The primate or human light chain framework region of the antibody selected for use with the light chain CDR sequences described herein, can have, for example, at least 70% (e.g., at least 75%, 80%, 85%, 90%, 95%, 98%, or at least 99%) identity with a light chain framework region of a non-human parent antibody. The primate or human antibody selected can have the same or substantially the same number of amino acids in its light chain complementarity determining regions to that of the light chain complementarity determining regions of any of the antibodies provided herein, e.g., any of the anti-transferrin receptor antibodies selected from Table 1. In some embodiments, the primate or human light chain framework region amino acid residues are from a natural primate or human antibody light chain framework region having at least 75% identity, at least 80% identity, at least 85% identity, at least 90% identity, at least 95% identity, at least 98% identity, at least 99% (or more) identity with the light chain framework regions of any anti-transferrin receptor antibody, such as any one of the anti-transferrin receptor antibodies selected from Table 1. In some embodiments, an anti-transferrin receptor antibody further comprises one, two, three or all four VL framework regions derived from a human light chain variable kappa subfamily. In some embodiments, an anti-transferrin receptor antibody further comprises one, two, three or all four VL framework regions derived from a human light chain variable lambda subfamily.

In some embodiments, any of the anti-transferrin receptor antibodies provided herein comprise a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. It should be appreciated that any of the light chain constant regions provided herein may be variants of any of the light chain constant regions provided herein. In some embodiments, the light chain constant region comprises an amino acid sequence that is at least 75%, 80%, 85%, 90%, 95%, 98%, or 99% identical to any of the light chain constant regions of any anti-transferrin receptor antibody, such as any one of the anti-transferrin receptor antibodies selected from Table 1.

In some embodiments, the anti-transferrin receptor antibody is any anti-transferrin receptor antibody, such as any one of the anti-transferrin receptor antibodies selected from Table 1.

In some embodiments, an anti-transferrin receptor antibody comprises a VL domain comprising the amino acid sequence of any anti-transferrin receptor antibody, such as any one of the anti-transferrin receptor antibodies selected from Table 1, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In some embodiments, an anti-transferrin receptor antibody comprises any of the VL domains, or VL domain variants, and any of the VH domains, or VH domain variants, wherein the VL and VH domains, or variants thereof, are from the same antibody clone, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between 5 μM and 500 nM, e.g., between 50 μM and 100 nM, e.g., between 500 μM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 μM or lower, 50 μM or lower, or 5 μM or lower). The affinity and binding kinetics of the anti-transferrin receptor antibody can be tested using any suitable method including but not limited to biosensor technology (e.g., OCTET or BIACORE).

In some embodiments, an antibody of the disclosure can bind to a target antigen (e.g., transferrin receptor) with relatively high affinity, e.g., with a K_{D} less than 10⁻⁶ M, 10⁻⁷ M, 10⁻⁸ M, 10⁻⁹ M, 10⁻¹⁰ M, 10⁻¹¹ M or lower. For example, anti-transferrin receptor antibodies can bind to a transferrin receptor protein (e.g., human transferrin receptor) with an affinity between μM and 500 nM, e.g., between 50 μM and 100 nM, e.g., between 500 μM and 50 nM. The disclosure also includes antibodies that compete with any of the antibodies described herein for binding to a transferrin receptor protein (e.g., human transferrin receptor) and that have an affinity of 50 nM or lower (e.g., 20 nM or lower, 10 nM or lower, 500 μM or lower, 50 μM or lower, or 5 μM or lower). The affinity and binding kinetics of the anti-transferrin receptor antibody can be tested using any suitable method including but not limited to biosensor technology (e.g., OCTET or BIACORE).

In some embodiments, the muscle-targeting agent is a transferrin receptor antibody (e.g., an antibody and variants thereof as described in International Application Publication WO 2016/081643, incorporated herein by reference).

In some embodiments, the heavy chain and light chain CDRs of an example antibody according to different definition systems are provided in Table 2. The different definition systems, e.g., the Kabat definition, the Chothia definition, and/or the contact definition have been described. See, e.g., (e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

TABLE 2
Heavy chain and light chain CDRs of a transferrin receptor antibody

| CDRs | Kabat | Chothia | Contact |
|---|---|---|---|
| CDR-H1 | SYWMH | GYTFTSY | TSYWMH |
| | (SEQ ID NO: 17) | (SEQ ID NO: 23) | (SEQ ID NO: 25) |
| CDR-H2 | EINPTNGRTNYIEKFKS | NPTNGR | WIGEINPTNGRTN |
| | (SEQ ID NO: 18) | (SEQ ID NO: 24) | (SEQ ID NO: 26) |
| CDR-H3 | GTRAYHY | GTRAYHY | ARGTRA |
| | (SEQ ID NO: 19) | (SEQ ID NO: 19) | (SEQ ID NO: 27) |
| CDR-L1 | RASDNLYSNLA | RASDNLYSNLA | YSNLAWY |
| | (SEQ ID NO: 20) | (SEQ ID NO: 20) | (SEQ ID NO: 28) |
| CDR-L2 | DATNLAD | DATNLAD | LLVYDATNLA |
| | (SEQ ID NO: 21) | (SEQ ID NO: 21) | (SEQ ID NO: 29) |
| CDR-L3 | QHFWGTPLT | QHFWGTPLT | QHFWGTPL |
| | (SEQ ID NO: 22) | (SEQ ID NO: 22) | (SEQ ID NO: 30) |

The heavy chain variable domain (VH) and light chain variable domain sequences are for also provided:

VH
(SEQ ID NO: 33)
QVQLQQPGAELVKPGASVKLSCKASGYTFTSYWMHWVKQRPGQGLEWIG
EINPINGRTNYIEKFKSKATLTVDKSSSTAYMQLSSLTSEDSAVYYCAR
GTRAYHYWGQGTSVTVSS
VL
(SEQ ID NO: 34)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELK

In some embodiments, the transferrin receptor antibody of the present disclosure comprises a CDR-H1, a CDR-H2, and a CDR-H3 that are the same as the CDR-H1, CDR-H2, and CDR-H3 shown in Table 2. Alternatively or in addition, the transferrin receptor antibody of the present disclosure comprises a CDR-L1, a CDR-L2, and a CDR-L3 that are the same as the CDR-L1, CDR-L2, and CDR-L3 shown in Table 2.

In some embodiments, the transferrin receptor antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1, CDR-H2, and CDR-H3 as shown in Table 2. “Collectively” means that the total number of amino acid variations in all of the three heavy chain CDRs is within the defined range. Alternatively or in addition, the transferrin receptor antibody of the present disclosure may comprise a CDR-L1, a CDR-L2, and a CDR-L3, which collectively contains no more than 5 amino acid variations (e.g., no more than 5, 4, 3, 2 or 1 amino acid variation) as compared with the CDR-L1, CDR-L2, and CDR-L3 as shown in Table 2.

In some embodiments, the transferrin receptor antibody of the present disclosure comprises a CDR-H1, a CDR-H2, and a CDR-H3, at least one of which contains no more than 3 amino acid variations (e.g., no more than 3, 2, or 1 amino acid variation) as compared with the counterpart heavy chain CDR as shown in Table 2. Alternatively or in addition, the transferrin receptor antibody of the present disclosure may comprise CDR-L1, a CDR-L2, and a CDR-L3, at least one of which contains no more than 3 amino acid variations (e.g., no more than 3, 2, or 1 amino acid variation) as compared with the counterpart light chain CDR as shown in Table 2.

In some embodiments, the transferrin receptor antibody of the present disclosure comprises a CDR-L3, which contains no more than 3 amino acid variations (e.g., no more than 3, 2, or 1 amino acid variation) as compared with the CDR-L3 as shown in Table 2. In some embodiments, the transferrin receptor antibody of the present disclosure comprises a CDR-L3 containing one amino acid variation as compared with the CDR-L3 as shown in Table 2. In some embodiments, the transferrin receptor antibody of the present disclosure comprises a CDR-L3 of QHFAGTPLT (SEQ ID NO: 31 according to the Kabat and Chothia definition system) or QHFAGTPL (SEQ ID NO: 32 according to the Contact definition system). In some embodiments, the transferrin receptor antibody of the present disclosure comprises a CDR-H1, a CDR-H2, a CDR-H3, a CDR-L1 and a CDR-L2 that are the same as the CDR-H1, CDR-H2, and CDR-H3 shown in Table 2, and comprises a CDR-L3 of QHFAGTPLT (SEQ ID NO: 31 according to the Kabat and Chothia definition system) or QHFAGTPL (SEQ ID NO: 32 according to the Contact definition system).

In some embodiments, the transferrin receptor antibody of the present disclosure comprises heavy chain CDRs that collectively are at least 80% (e.g., 80%, 85%, 90%, 95%, or 98%) identical to the heavy chain CDRs as shown in Table 2. Alternatively or in addition, the transferrin receptor antibody of the present disclosure comprises light chain CDRs that collectively are at least 80% (e.g., 80%, 85%, 90%, 95%, or 98%) identical to the light chain CDRs as shown in Table 2.

In some embodiments, the transferrin receptor antibody of the present disclosure comprises a VH comprising the amino acid sequence of SEQ ID NO: 33. Alternatively or in addition, the transferrin receptor antibody of the present disclosure comprises a VL comprising the amino acid sequence of SEQ ID NO: 34.

In some embodiments, the transferrin receptor antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 33. Alternatively or in addition, the transferrin receptor antibody of the present disclosure comprises a VL containing no more than amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 34.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 33. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 34.

In some embodiments, the transferrin receptor antibody of the present disclosure is a humanized antibody (e.g., a humanized variant containing one or more CDRs of Table 2). In some embodiments, the transferrin receptor antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 2, and comprises a humanized heavy chain variable region and/or a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 2) into the IGKV1-NL1*01 and IGHV1-3*01 human variable domains. In some embodiments, the transferrin receptor antibody of the present disclosure is a humanized variant comprising one or more amino acid substitutions at positions 9, 13, 17, 18, 40, 45, and 70 as compared with the VL as set forth in SEQ ID NO: 34, and/or one or more amino acid substitutions at positions 1, 5, 7, 11, 12, 20, 38, 40, 44, 66, 75, 81, 83, 87, and 108 as compared with the VH as set forth in SEQ ID NO: 33. In some embodiments, the transferrin receptor antibody of the present disclosure is a humanized variant comprising amino acid substitutions at all of positions 9, 13, 17, 18, 40, 45, and 70 as compared with the VL as set forth in SEQ ID NO: 34, and/or amino acid substitutions at all of positions 1, 5, 7, 11, 12, 20, 38, 40, 44, 66, 75, 81, 83, 87, and 108 as compared with the VH as set forth in SEQ ID NO: 33.

In some embodiments, the transferrin receptor antibody of the present disclosure is a humanized antibody and contains the residues at positions 43 and 48 of the VL as set forth in SEQ ID NO: 34. Alternatively or in addition, the transferrin receptor antibody of the present disclosure is a humanized antibody and contains the residues at positions 48, 67, 69, 71, and 73 of the VH as set forth in SEQ ID NO: 33.

The VH and VL amino acid sequences of an example humanized antibody that may be used in accordance with the present disclosure are provided:

Humanized VH
(SEQ ID NO: 35)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSS
Humanized VL
(SEQ ID NO: 36)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIK

In some embodiments, the transferrin receptor antibody of the present disclosure comprises a VH comprising the amino acid sequence of SEQ ID NO: 35. Alternatively or in addition, the transferrin receptor antibody of the present disclosure comprises a VL comprising the amino acid sequence of SEQ ID NO: 36.

In some embodiments, the transferrin receptor antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 35. Alternatively or in addition, the transferrin receptor antibody of the present disclosure comprises a VL containing no more than amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 36.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 35. Alternatively or in addition, the transferrin receptor antibody of the present disclosure comprises a VL comprising an amino acid sequence that is at least 80% (e.g., 80%, 85%, 90%, 95%, or 98%) identical to the VL as set forth in SEQ ID NO: 36.

In some embodiments, the transferrin receptor antibody of the present disclosure is a humanized variant comprising amino acid substitutions at one or more of positions 43 and 48 as compared with the VL as set forth in SEQ ID NO: 34, and/or amino acid substitutions at one or more of positions 48, 67, 69, 71, and 73 as compared with the VH as set forth in SEQ ID NO: 33. In some embodiments, the transferrin receptor antibody of the present disclosure is a humanized variant comprising a S43A and/or a V48L mutation as compared with the VL as set forth in SEQ ID NO: 34, and/or one or more of A67V, L69I, V71R, and K73T mutations as compared with the VH as set forth in SEQ ID NO: 33

In some embodiments, the transferrin receptor antibody of the present disclosure is a humanized variant comprising amino acid substitutions at one or more of positions 9, 13, 17, 18, 40, 43, 48, 45, and 70 as compared with the VL as set forth in SEQ ID NO: 34, and/or amino acid substitutions at one or more of positions 1, 5, 7, 11, 12, 20, 38, 40, 44, 48, 66, 67, 69, 71, 73, 75, 81, 83, 87, and 108 as compared with the VH as set forth in SEQ ID NO: 33.

In some embodiments, the transferrin receptor antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the transferrin receptor antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the heavy chain of any of the transferrin receptor antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An exemplary human IgG1 constant region is given below:

(SEQ ID NO: 37)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the transferrin receptor antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 38)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php., both of which are incorporated by reference herein.

Exemplary heavy chain and light chain amino acid sequences of the transferrin receptor antibodies described are provided below:

Heavy Chain (VH + human IgG1 constant region)
(SEQ ID NO: 39)
QVQLQQPGAELVKPGASVKLSCKASGYTFTSYWMHWVKQRPGQGLEWIG
EINPINGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (VL + kappa light chain)
(SEQ ID NO: 40)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Heavy Chain (humanized VH + human IgG1 constant
region)
(SEQ ID NO: 41)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK
Light Chain (humanized VL + kappa light chain)
(SEQ ID NO: 42)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the transferrin receptor antibody described herein comprises a heavy chain comprising an amino acid sequence that is at least 80% (e.g., 80%, 85%, 90%, 95%, or 98%) identical to SEQ ID NO: 39. Alternatively or in addition, the transferrin receptor antibody described herein comprises a light chain comprising an amino acid sequence that is at least 80% (e.g., 80%, 85%, 90%, 95%, or 98%) identical to SEQ ID NO: 40. In some embodiments, the transferrin receptor antibody described herein comprises a heavy chain comprising the amino acid sequence of SEQ ID NO: 39. Alternatively or in addition, the transferrin receptor antibody described herein comprises a light chain comprising the amino acid sequence of SEQ ID NO: 40.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 40.

In some embodiments, the transferrin receptor antibody described herein comprises a heavy chain comprising an amino acid sequence that is at least 80% (e.g., 80%, 85%, 90%, 95%, or 98%) identical to SEQ ID NO: 41. Alternatively or in addition, the transferrin receptor antibody described herein comprises a light chain comprising an amino acid sequence that is at least 80% (e.g., 80%, 85%, 90%, 95%, or 98%) identical to SEQ ID NO: 42. In some embodiments, the transferrin receptor antibody described herein comprises a heavy chain comprising the amino acid sequence of SEQ ID NO: 41. Alternatively or in addition, the transferrin receptor antibody described herein comprises a light chain comprising the amino acid sequence of SEQ ID NO: 42.

In some embodiments, the transferrin receptor antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain of a humanized sequence as set forth in SEQ ID NO: 39. Alternatively or in addition, the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain of a humanized sequence as set forth in SEQ ID NO: 40.

In some embodiments, the transferrin receptor antibody is an antigen binding fragment (FAB) of an intact antibody (full-length antibody). Antigen binding fragment of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Exemplary FAB s amino acid sequences of the transferrin receptor antibodies described herein are provided below:

Heavy Chain FAB (VH + a portion of human IgG1
constant region)
(SEQ ID NO: 43)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP
Heavy Chain FAB (humanized VH + a portion of
human IgG1 constant region)
(SEQ ID NO: 44)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP

The transferrin receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the transferrin receptor antibody described herein is a scFv. In some embodiments, the transferrin receptor antibody described herein is a scFv-Fab (e.g., scFv fused to a portion of a constant region). In some embodiments, the transferrin receptor antibody described herein is a scFv fused to a constant region (e.g., human IgG1 constant region as set forth in SEQ ID NO: 39).

b. Other Muscle-Targeting Antibodies

In some embodiments, the muscle-targeting antibody is an antibody that specifically binds hemojuvelin, caveolin-3, Duchenne muscular dystrophy peptide, myosin Iib, or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fey receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 6) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 6). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 7) was identified and this muscle-targeting peptide showed improved binding to C2C12 cells relative to the ASSLNIA (SEQ ID NO: 6) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 8) appeared most frequently. Accordingly, in some embodiments, the muscle-targeting agent comprises the amino acid sequence TARGEHKEEELI (SEQ ID NO: 8).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 9), CSERSMNFC (SEQ ID NO: 10), CPKTRRVPC (SEQ ID NO: 11), WLSEAGPVVTVRALRGTGSW (SEQ ID NO: 12), ASSLNIA (SEQ ID NO: 6), CMQHSMRVC (SEQ ID NO: 13), and DDTRHWG (SEQ ID NO: 14). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10-15 kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LATS transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

B. Molecular Payloads

Some aspects of the disclosure provide molecular payloads, e.g., for modulating a biological outcome, e.g., the transcription of a DNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of muscle-targeting agents may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a DMPK allele comprising a disease-associated-repeat expansion. Exemplary molecular payloads are described in further detail herein, however, it should be appreciated that the exemplary molecular payloads provided herein are not meant to be limiting.

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

Examples of oligonucleotides useful for targeting DMPK are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein in their entireties.

Examples of oligonucleotides for promoting DMPK gene editing include US

Patent Application Publication 20170088819A1, published on Mar. 3, 2017, entitled “Genetic Correction Of Myotonic Dystrophy Type 1”; and International Patent Application Publication WO18002812A1, published on Apr. 1, 2018, entitled “Materials And Methods For Treatment Of Myotonic Dystrophy Type 1 (DM1) And Other Related Disorders,” the contents of each of which are incorporated herein in their entireties.

In some embodiments, oligonucleotides may have a region of complementarity to a sequence set forth as follows, which is an example human DMPK gene sequence (Gene ID 1760; NM_001081560.2):

(SEQ ID NO: 15)
AGGGGGGCTGGACCAAGGGGTGGGGAGAAGGGGAGGAGGCCTCGGCCGG
CCGCAGAGAGAAGTGGCCAGAGAGGCCCAGGGGACAGCCAGGGACAGGC
AGACATGCAGCCAGGGCTCCAGGGCCTGGACAGGGGCTGCCAGGCCCTG
TGACAGGAGGACCCCGAGCCCCCGGCCCGGGGAGGGGCCATGGTGCTGC
CTGTCCAACATGTCAGCCGAGGTGCGGCTGAGGCGGCTCCAGCAGCTGG
TGTTGGACCCGGGCTTCCTGGGGCTGGAGCCCCTGCTCGACCTTCTCCT
GGGCGTCCACCAGGAGCTGGGCGCCTCCGAACTGGCCCAGGACAAGTAC
GTGGCCGACTTCTTGCAGTGGGCGGAGCCCATCGTGGTGAGGCTTAAGG
AGGTCCGACTGCAGAGGGACGACTTCGAGATTCTGAAGGTGATCGGACG
CGGGGCGTTCAGCGAGGTAGCGGTAGTGAAGATGAAGCAGACGGGCCAG
GTGTATGCCATGAAGATCATGAACAAGTGGGACATGCTGAAGAGGGGCG
AGGTGTCGTGCTTCCGTGAGGAGAGGGACGTGTTGGTGAATGGGGACCG
GCGGTGGATCACGCAGCTGCACTTCGCCTTCCAGGATGAGAACTACCTG
TACCTGGTCATGGAGTATTACGTGGGGGGGACCTGCTGACACTGCTGAG
CAAGTTTGGGGAGCGGATTCCGGCCGAGATGGCGCGCTTCTACCTGGCG
GAGATTGTCATGGCCATAGACTCGGTGCACCGGCTTGGCTACGTGCACA
GGGACATCAAACCCGACAACATCCTGCTGGACCGCTGTGGCCACATCCG
CCTGGCCGACTTCGGCTCTTGCCTCAAGCTGCGGGCAGATGGAACGGTG
CGGTCGCTGGTGGCTGTGGGCACCCCAGACTACCTGTCCCCCGAGATCC
TGCAGGCTGTGGGCGGTGGGCCTGGGACAGGCAGCTACGGGCCCGAGTG
TGACTGGTGGGCGCTGGGTGTATTCGCCTATGAAATGTTCTATGGGCAG
ACGCCCTTCTACGCGGATTCCACGGCGGAGACCTATGGCAAGATCGTCC
ACTACAAGGAGCACCTCTCTCTGCCGCTGGTGGACGAAGGGGTCCCTGA
GGAGGCTCGAGACTTCATTCAGCGGTTGCTGTGTCCCCCGGAGACACGG
CTGGGCCGGGGTGGAGCAGGCGACTTCCGGACACATCCCTTCTTCTTTG
GCCTCGACTGGGATGGTCTCCGGGACAGCGTGCCCCCCTTTACACCGGA
TTTCGAAGGTGCCACCGACACATGCAACTTCGACTTGGTGGAGGACGGG
CTCACTGCCATGGAGACACTGTCGGACATTCGGGAAGGTGCGCCGCTAG
GGGTCCACCTGCCTTTTGTGGGCTACTCCTACTCCTGCATGGCCCTCAG
GGACAGTGAGGTCCCAGGCCCCACACCCATGGAACTGGAGGCCGAGCAG
CTGCTTGAGCCACACGTGCAAGCGCCCAGCCTGGAGCCCTCGGTGTCCC
CACAGGATGAAACAGCTGAAGTGGCAGTTCCAGCGGCTGTCCCTGCGGC
AGAGGCTGAGGCCGAGGTGACGCTGCGGGAGCTCCAGGAAGCCCTGGAG
GAGGAGGTGCTCACCCGGCAGAGCCTGAGCCGGGAGATGGAGGCCATCC
GCACGGACAACCAGAACTTCGCCAGTCAACTACGCGAGGCAGAGGCTCG
GAACCGGGACCTAGAGGCACACGTCCGGCAGTTGCAGGAGCGGATGGAG
TTGCTGCAGGCAGAGGGAGCCACAGCTGTCACGGGGGTCCCCAGTCCCC
GGGCCACGGATCCACCTTCCCATCTAGATGGCCCCCCGGCCGTGGCTGT
GGGCCAGTGCCCGCTGGTGGGGCCAGGCCCCATGCACCGCCGCCACCTG
CTGCTCCCTGCCAGGGTCCCTAGGCCTGGCCTATCGGAGGCGCTTTCCC
TGCTCCTGTTCGCCGTTGTTCTGTCTCGTGCCGCCGCCCTGGGCTGCAT
TGGGTTGGTGGCCCACGCCGGCCAACTCACCGCAGTCTGGCGCCGCCCA
GGAGCCGCCCGCGCTCCCTGAACCCTAGAACTGTCTTCGACTCCGGGGC
CCCGTTGGAAGACTGAGTGCCCGGGGCACGGCACAGAAGCCGCGCCCAC
CGCCTGCCAGTTCACAACCGCTCCGAGCGTGGGTCTCCGCCCAGCTCCA
GTCCTGTGATCCGGGCCCGCCCCCTAGCGGCCGGGGAGGGAGGGGCCGG
GTCCGCGGCCGGCGAACGGGGCTCGAAGGGTCCTTGTAGCCGGGAATGC
TGCTGCTGCTGCTGCTGCTGCTGCTGCTGCTGCTGCTGCTGCTGCTGCT
GCTGCTGCTGGGGGGATCACAGACCATTTCTTTCTTTCGGCCAGGCTGA
GGCCCTGACGTGGATGGGCAAACTGCAGGCCTGGGAAGGCAGCAAGCCG
GGCCGTCCGTGTTCCATCCTCCACGCACCCCCACCTATCGTTGGTTCGC
AAAGTGCAAAGCTTTCTTGTGCATGACGCCCTGCTCTGGGGAGCGTCTG
GCGCGATCTCTGCCTGCTTACTCGGGAAATTTGCTTTTGCCAAACCCGC
TTTTTCGGGGATCCCGCGCCCCCCTCCTCACTTGCGCTGCTCTCGGAGC
CCCAGCCGGCTCCGCCCGCTTCGGCGGTTTGGATATTTATTGACCTCGT
CCTCCGACTCGCTGACAGGCTACAGGACCCCCAACAACCCCAATCCACG
TTTTGGATGCACTGAGACCCCGACATTCCTCGGTATTTATTGTCTGTCC
CCACCTAGGACCCCCACCCCCGACCCTCGCGAATAAAAGGCCCTCCATC
TGCCCAAAGCTCTGGA.

In some embodiments, oligonucleotides may have a region of complementarity to a sequence set forth as follows, which is an example mouse DMPK gene sequence (Gene ID 13400; NM_001190490.1).

(SEQ ID NO: 16)
GAACTGGCCAGAGAGACCCAAGGGATAGTCAGGGACGGGCAGACATGCA
GCTAGGGTTCTGGGGCCTGGACAGGGGCAGCCAGGCCCTGTGACGGGAA
GACCCCGAGCTCCGGCCCGGGGAGGGGCCATGGTGTTGCCTGCCCAACA
TGTCAGCCGAAGTGCGGCTGAGGCAGCTCCAGCAGCTGGTGCTGGACCC
AGGCTTCCTGGGACTGGAGCCCCTGCTCGACCTTCTCCTGGGCGTCCAC
CAGGAGCTGGGTGCCTCTCACCTAGCCCAGGACAAGTATGTGGCCGACT
TCTTGCAGTGGGTGGAGCCCATTGCAGCAAGGCTTAAGGAGGTCCGACT
GCAGAGGGATGATTTTGAGATTTTGAAGGTGATCGGGCGTGGGGCGTTC
AGCGAGGTAGCGGTGGTGAAGATGAAACAGACGGGCCAAGTGTATGCCA
TGAAGATTATGAATAAGTGGGACATGCTGAAGAGAGGCGAGGTGTCGTG
CTTCCGGGAAGAAAGGGATGTATTAGTGAAAGGGGACCGGCGCTGGATC
ACACAGCTGCACTTTGCCTTCCAGGATGAGAACTACCTGTACCTGGTCA
TGGAATACTACGTGGGCGGGGACCTGCTAACGCTGCTGAGCAAGTTTGG
GGAGCGGATCCCCGCCGAGATGGCTCGCTTCTACCTGGCCGAGATTGTC
ATGGCCATAGACTCCGTGCACCGGCTGGGCTACGTGCACAGGGACATCA
AACCAGATAACATTCTGCTGGACCGATGTGGGCACATTCGCCTGGCAGA
CTTCGGCTCCTGCCTCAAACTGCAGCCTGATGGAATGGTGAGGTCGCTG
GTGGCTGTGGGCACCCCGGACTACCTGTCTCCTGAGATTCTGCAGGCCG
TTGGTGGAGGGCCTGGGGCAGGCAGCTACGGGCCAGAGTGTGACTGGTG
GGCACTGGGCGTGTTCGCCTATGAGATGTTCTATGGGCAGACCCCCTTC
TACGCGGACTCCACAGCCGAGACATATGCCAAGATTGTGCACTACAGGG
AACACTTGTCGCTGCCGCTGGCAGACACAGTTGTCCCCGAGGAAGCTCA
GGACCTCATTCGTGGGCTGCTGTGTCCTGCTGAGATAAGGCTAGGTCGA
GGTGGGGCAGACTTCGAGGGTGCCACGGACACATGCAATTTCGATGTGG
TGGAGGACCGGCTCACTGCCATGGTGAGCGGGGGCGGGGAGACGCTGTC
AGACATGCAGGAAGACATGCCCCTTGGGGTGCGCCTGCCCTTCGTGGGC
TACTCCTACTGCTGCATGGCCTTCAGAGACAATCAGGTCCCGGACCCCA
CCCCTATGGAACTAGAGGCCCTGCAGTTGCCTGTGTCAGACTTGCAAGG
GCTTGACTTGCAGCCCCCAGTGTCCCCACCGGATCAAGTGGCTGAAGAG
GCTGACCTAGTGGCTGTCCCTGCCCCTGTGGCTGAGGCAGAGACCACGG
TAACGCTGCAGCAGCTCCAGGAAGCCCTGGAAGAAGAGGTTCTCACCCG
GCAGAGCCTGAGCCGCGAGCTGGAGGCCATCCGGACCGCCAACCAGAAC
TTCTCCAGCCAACTACAGGAGGCCGAGGTCCGAAACCGAGACCTGGAGG
CGCATGTTCGGCAGCTACAGGAACGGATGGAGATGCTGCAGGCCCCAGG
AGCCGCAGCCATCACGGGGGTCCCCAGTCCCCGGGCCACGGATCCACCT
TCCCATCTAGATGGCCCCCCGGCCGTGGCTGTGGGCCAGTGCCCGCTGG
TGGGGCCAGGCCCCATGCACCGCCGTCACCTGCTGCTCCCTGCCAGGAT
CCCTAGGCCTGGCCTATCCGAGGCGCGTTGCCTGCTCCTGTTCGCCGCT
GCTCTGGCTGCTGCCGCCACACTGGGCTGCACTGGGTTGGTGGCCTATA
CCGGCGGTCTCACCCCAGTCTGGTGTTTCCCGGGAGCCACCTTCGCCCC
CTGAACCCTAAGACTCCAAGCCATCTTTCATTTAGGCCTCCTAGGAAGG
TCGAGCGACCAGGGAGCGACCCAAAGCGTCTCTGTGCCCATCGCGCCCC
CCCCCCCCCCCCACCGCTCCGCTCCACACTTCTGTGAGCCTGGGTCCCC
ACCCAGCTCCGCTCCTGTGATCCAGGCCTGCCACCTGGCGGCCGGGGAG
GGAGGAACAGGGCTCGTGCCCAGCACCCCTGGTTCCTGCAGAGCTGGTA
GCCACCGCTGCTGCAGCAGCTGGGCATTCGCCGACCTTGCTTTACTCAG
CCCCGACGTGGATGGGCAAACTGCTCAGCTCATCCGATTTCACTTTTTC
ACTCTCCCAGCCATCAGTTACAAGCCATAAGCATGAGCCCCCTATTTCC
AGGGACATCCCATTCCCATAGTGATGGATCAGCAAGACCTCTGCCAGCA
CACACGGAGTCTTTGGCTTCGGACAGCCTCACTCCTGGGGGTTGCTGCA
ACTCCTTCCCCGTGTACACGTCTGCACTCTAACAACGGAGCCACAGCTG
CACTCCCCCCTCCCCCAAAGCAGTGTGGGTATTTATTGATCTTGTTATC
TGACTCACTGACAGACTCCGGGACCCACGTTTTAGATGCATTGAGACTC
GACATTCCTCGGTATTTATTGTCTGTCCCCACCTACGACCTCCACTCCC
GACCCTTGCGAATAAAATACTTCTGGTCTGCCCTAAA.

In some embodiments, an oligonucleotide may have a region of complementarity to DMPK gene sequences of multiple species, e.g., selected from human, mouse and non-human species.

In some embodiments, the oligonucleotide may have region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46.; and Machuca-Tzili L. et al. “Clinical and molecular aspects of the myotonic dystrophies: a review.” Muscle Nerve. 2005 July; 32(1):1-18.; the contents of each of which are incorporated herein by reference in their entireties.

In some embodiments, the oligonucleotide may target lncRNA or mRNA, e.g., for degradation. In some embodiments, the oligonucleotide may target, e.g., for degradation, a nucleic acid encoding a protein involved in a mismatch repair pathway, e.g., MSH2, MutLalpha, MutSbeta, MutLalpha. Non-limiting examples of proteins involved in mismatch repair pathways, for which mRNAs encoding such proteins may be targeted by oligonucleotides described herein, are described in Iyer, R. R. et al., “DNA triplet repeat expansion and mismatch repair” Annu Rev Biochem. 2015; 84:199-226.; and Schmidt M. H. and Pearson C. E., “Disease-associated repeat instability and mismatch repair” DNA Repair (Amst). 2016 February; 38:117-26.

In some embodiments, an oligonucleotide provided herein is an antisense oligonucleotide targeting DMPK. In some embodiments, the oligonucleotide targeting is any one of the antisense oligonucleotides (e.g., a Gapmer) targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference). In some embodiments, the DMPK targeting oligonucleotide targets a region of the DMPK gene sequence as set forth in Genbank accession No. NM_001081560.2 (SEQ ID NO: 15) or as set forth in Genbank accession No. NG_009784.1.

In some embodiments, the DMPK targeting oligonucleotide comprises a nucleotide sequence comprising a region complementary to a target region that is at least 10 continuous nucleotides (e.g., at least 10, at least 12, at least 14, at least 16, or more continuous nucleotides) in SEQ ID NO: 15.

In some embodiments, the DMPK targeting oligonucleotide comprise a gapmer motif. “Gapmer” means a chimeric antisense compound in which an internal region having a plurality of nucleotides that support RNase H cleavage is positioned between external regions having one or more nucleotides, wherein the nucleotides comprising the internal region are chemically distinct from the nucleotide or nucleotides comprising the external regions. The internal region can be referred to as a “gap segment” and the external regions can be referred to as “wing segments.” In some embodiments, the DMPK targeting oligonucleotide comprises one or more modified nucleotides, and/or one or more modified internucleotide linkages In some embodiments, the internucleotide linkage is a phosphorothioate linkage In some embodiments, the oligonucleotide comprises a full phosphorothioate backbone. In some embodiments, the oligonucleotide is a DNA gapmer with cET ends (e.g., 3-10-3; cET-DNA-cET). In some embodiments, the DMPK targeting oligonucleotide comprises one or more 6′-(S)—CH₃ biocyclic nucleotides, one or more β-D-2′-deoxyribonucleotides, and/or one or more 5-methylcytosine nucleotides.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an target nucleic acid. In some embodiments a complementary nucleotide sequence need not be 100% complementary to that of its target to be specifically hybridizable or specific for a target nucleic acid.

In some embodiments, an oligonucleotide comprises region of complementarity to a target nucleic acid that is in the range of 8 to 15, 8 to 30, 8 to 40, or 10 to 50, or 5 to 50, or to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, an oligonucleotide comprises at least 10, 11, 12, 13, 14, 15, 16, 17, 18, 19, or 20 consecutive nucleotides of a sequence comprising any one of SEQ ID NO: 45-280. In some embodiments, an oligonucleotide comprises a sequence comprising any one of SEQ ID NO: 45-280. In some embodiments, an oligonucleotide comprises a sequence that shares at least 70%, 75%, 80%, 85%, 90%, 95%, or 97% sequence identity with at least 12 or at least 15 consecutive nucleotides of any one of SEQ ID NO: 45-280.

In some embodiments, an oligonucleotide comprises a sequence that targets a DMPK sequence comprising any one of SEQ ID NO: 281-516. In some embodiments, an oligonucleotide comprises at least 10, 11, 12, 13, 14, 15, 16, 17, 18, 19, or 20 nucleotides (e.g., consecutive nucleotides) that are complementary to a DMPK sequence comprising any one of SEQ ID NO: 281-516. In some embodiments, an oligonucleotide comprises a sequence that is at least 70%, 75%, 80%, 85%, 90%, 95%, or 97% complementary with at least 12 or at least 15 consecutive nucleotides of any one of SEQ ID NO: 281-516.

In some embodiments, the oligonucleotide is complementary (e.g., at least 85% at least 90%, at least 95%, or 100%) to a target sequence of any one of the oligonucleotides provided herein (e.g., the oligonucleotides listed in Table 4). In some embodiments, such target sequence is 100% complementary to the oligonucleotide listed in Table 4.

In some embodiments, any one or more of the thymine bases (T's) in any of the oligonucleotides provided herein (e.g., the oligonucleotides listed in Table 4) may optionally be uracil bases (U's), and/or any one or more of the U's may optionally be T's.

b. Oligonucleotide Modifications:

The oligonucleotides described herein may be modified, e.g., comprise a modified sugar moiety, a modified internucleotide linkage, a modified nucleotide and/or combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleotide linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

c. Modified Nucleotides

In some embodiments, an oligonucleotide include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA).

In some embodiments, an oligonucleotide can include at least one 2′-O-methyl-modified nucleotide, and in some embodiments, all of the nucleotides include a 2′-O-methyl modification. In some embodiments, an oligonucleotide comprises modified nucleotides in which the ribose ring comprises a bridge moiety connecting two atoms in the ring, e.g., connecting the 2′-O atom to the 4′-C atom. In some embodiments, the oligonucleotides are “locked,” e.g., comprise modified nucleotides in which the ribose ring is “locked” by a methylene bridge connecting the 2′-O atom and the 4′-C atom. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety.

Other modifications that may be used in the oligonucleotides disclosed herein include ethylene-bridged nucleic acids (ENAs). ENAs include, but are not limited to, 2′-0,4′-C-ethylene-bridged nucleic acids. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties.

In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. In some embodiments, the oligonucleotide comprises a modified nucleotide disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA.

In some embodiments, the oligonucleotide may have at least one modified nucleotide that results in an increase in Tm of the oligonucleotide in a range of 1° C., 2° C., 3° C., 4° C., or 5° C. compared with an oligonucleotide that does not have the at least one modified nucleotide. The oligonucleotide may have a plurality of modified nucleotides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleotide.

The oligonucleotide may comprise alternating nucleotides of different kinds. For example, an oligonucleotide may comprise alternating deoxyribonucleotides or ribonucleotides and 2′-fluoro-deoxyribonucleotides. An oligonucleotide may comprise alternating deoxyribonucleotides or ribonucleotides and 2′-O-methyl nucleotides. An oligonucleotide may comprise alternating 2′-fluoro nucleotides and 2′-O-methyl nucleotides. An oligonucleotide may comprise alternating bridged nucleotides and 2′-fluoro or 2′-O-methyl nucleotides.

d. Internucleotide Linkages/Backbones

In some embodiments, oligonucleotide may contain a phosphorothioate or other modified internucleotide linkage. In some embodiments, the oligonucleotide comprises phosphorothioate internucleotide linkages. In some embodiments, the oligonucleotide comprises phosphorothioate internucleotide linkages between at least two nucleotides. In some embodiments, the oligonucleotide comprises phosphorothioate internucleotide linkages between all nucleotides. For example, in some embodiments, oligonucleotides comprise modified internucleotide linkages at the first, second, and/or third internucleotide linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleotide linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, the oligonucleotide is a gapmer. A gapmer oligonucleotide generally has the formula 5′-X—Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., one to six modified nucleotides. Examples of modified nucleotides include, but are not limited to, 2′ MOE or 2′OMe or Locked Nucleic Acid bases (LNA). The flanking sequences X and Z may be of one to twenty nucleotides, one to eight nucleotides or one to five nucleotides in length, in some embodiments. The flanking sequences X and Z may be of similar length or of dissimilar lengths. The gap-segment Y may be a nucleotide sequence of five to twenty nucleotides, size to twelve nucleotides or six to ten nucleotides in length, in some embodiments.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleotides. In some embodiments, one or both flanking regions each independently comprise one or more phosphorothioate internucleotide linkages (e.g., phosphorothioate internucleotide linkages or other linkages) between at least two, at least three, at least four, at least five or more nucleotides. In some embodiments, the gap region and two flanking regions each independently comprise modified internucleotide linkages (e.g., phosphorothioate internucleotide linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

i. Mixmers

In some embodiments, an oligonucleotide described herein may be a mixmer or comprise a mixmer sequence pattern. In general, mixmers are oligonucleotides that comprise both naturally and non-naturally occurring nucleotides or comprise two different types of non-naturally occurring nucleotides typically in an alternating pattern. Mixmers generally have higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule. Such oligonucleotides that are incapable of recruiting RNAse H have been described, for example, see WO2007/112754 or WO2007/112753.

In some embodiments, the mixmer comprises or consists of a repeating pattern of nucleotide analogues and naturally occurring nucleotides, or one type of nucleotide analogue and a second type of nucleotide analogue. However, a mixmer need not comprise a repeating pattern and may instead comprise any arrangement of modified nucleotides and naturally occurring nucleotides or any arrangement of one type of modified nucleotide and a second type of modified nucleotide. The repeating pattern, may, for instance be every second or every third nucleotide is a modified nucleotide, such as LNA, and the remaining nucleotides are naturally occurring nucleotides, such as DNA, or are a 2′ substituted nucleotide analogue such as 2′MOE or 2′ fluoro analogues, or any other modified nucleotide described herein. It is recognized that the repeating pattern of modified nucleotide, such as LNA units, may be combined with modified nucleotide at fixed positions—e.g. at the 5′ or 3′ termini.

In some embodiments, a mixmer does not comprise a region of more than 5, more than 4, more than 3, or more than 2 consecutive naturally occurring nucleotides, such as DNA nucleotides. In some embodiments, the mixmer comprises at least a region consisting of at least two consecutive modified nucleotide, such as at least two consecutive LNAs. In some embodiments, the mixmer comprises at least a region consisting of at least three consecutive modified nucleotide units, such as at least three consecutive LNAs.

In some embodiments, the mixmer does not comprise a region of more than 7, more than 6, more than 5, more than 4, more than 3, or more than 2 consecutive nucleotide analogues, such as LNAs. In some embodiments, LNA units may be replaced with other nucleotide analogues, such as those referred to herein.

Mixmers may be designed to comprise a mixture of affinity enhancing modified nucleotides, such as in non-limiting example LNA nucleotides and 2′-O-methyl nucleotides. In some embodiments, a mixmer comprises modified internucleotide linkages (e.g., phosphorothioate internucleotide linkages or other linkages) between at least two, at least three, at least four, at least five or more nucleotides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7,687,617.

j. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791).

The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

l. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′, 5′-phosphate diester to a 2′, 3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences may be synthesized conventionally, for example, by using RNA polymerases such as T7 or SP6.

n. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid-programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn 1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via a oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

ii. Small Molecules:

Any suitable small molecule may be used as a molecular payload, as described herein. In some embodiments, the small molecule is as described in US Patent Application Publication 2016052914A1, published on Feb. 25, 2016, entitled “Compounds And Methods For Myotonic Dystrophy Therapy”. Further examples of small molecule payloads are provided in Lopez-Morato M, et al., Small Molecules Which Improve Pathogenesis of Myotonic Dystrophy Type 1, (Review) Front. Neurol., 18 May 2018. For example, in some embodiments, the small molecule is an MBNL1 upregulator such as phenylbuthazone, ketoprofen, ISOX, or vorinostat. In some embodiments, the small molecule is an H-Ras pathway inhibitor such as manumycin A. In some embodiments, the small molecule is a protein kinase modulator such as Ro-318220, C16, C51, Metformin, AICAR, lithium chloride, TDZD-8 or Bio. In some embodiments, the small molecule is a plant alkaloid such as harmine. In some embodiments, the small molecule is a transcription inhibitor such as pentamidine, propamidine, heptamidiine or actinomycin D. In some embodiments, the small molecule is an inhibitor of Glycogen synthase kinase 3 beta (GSK3B), for example, as disclosed in Jones K, et al., GSK3(3 mediates muscle pathology in myotonic dystrophy. J Clin Invest. 2012 December; 122(12):4461-72; and Wei C, et al., GSK3(3 is a new therapeutic target for myotonic dystrophy type 1. Rare Dis. 2013; 1: e26555; and Palomo V, et al., Subtly Modulating Glycogen Synthase Kinase 3 (3: Allosteric Inhibitor Development and Their Potential for the Treatment of Chronic Diseases. J Med Chem. 2017 Jun. 22; 60(12):4983-5001, the contents of each of which are incorporated herein by reference in their entireties. In some embodiments, the small molecule is a substituted pyrido[2,3-d]pyrimidines and pentamidine-like compound, as disclosed in Gonzalez A L, et al., In silico discovery of substituted pyrido[2,3-d]pyrimidines and pentamidine-like compounds with biological activity in myotonic dystrophy models. PLoS One. 2017 Jun. 5; 12(6):e0178931, the contents of which are incorporated herein by reference in its entirety. In some embodiments, the small molecule is an MBNL1 modulator, for example, as disclosed in: Zhange F, et al., A flow cytometry-based screen identifies MBNL1 modulators that rescue splicing defects in myotonic dystrophy type I. Hum Mol Genet. 2017 Aug. 15; 26(16):3056-3068, the contents of which are incorporated herein by reference in its entirety.

iii. Peptides

Any suitable peptide or protein may be used as a molecular payload, as described herein. A peptide or protein payload may correspond to a sequence of a protein that preferentially binds to a nucleic acid, e.g. a disease-associated repeat, or a protein, e.g. MBNL1, found in muscle cells. In some embodiments, peptides or proteins may be produced, synthesized, and/or derivatized using several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.).

In some embodiments, the peptide is as described in US Patent Application 2018/0021449, published on Jan. 25, 2018, “Antisense conjugates for decreasing expression of DMPK”. In some embodiments, the peptide is as described in Garcia-Lopez et al., “In vivo discovery of a peptide that prevents CUG—RNA hairpin formation and reverses RNA toxicity in myotonic dystrophy models”, PNAS Jul. 19, 2011. 108 (29) 11866-11871. In some embodiments, the peptide or protein may target, e.g., bind to, a disease-associated repeat, e.g. a RNA CUG repeat expansion.

In some embodiments, the peptide or protein comprises a fragment of an MBNL protein, e.g., MBNL1. In some embodiments, the peptide or protein comprises at least one zinc finger. In some embodiments, the peptide or protein may comprise about 2-25 amino acids, about 2-20 amino acids, about 2-15 amino acids, about 2-10 amino acids, or about 2-5 amino acids. The peptide or protein may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, the peptide may be linear; in other embodiments, the peptide may be cyclic, e.g. bicyclic.

iv. Nucleic Acid Constructs

Any suitable gene expression construct may be used as a molecular payload, as described herein. In some embodiments, a gene expression construct may be a vector or a cDNA fragment. In some embodiments, a gene expression construct may be messenger RNA (mRNA). In some embodiments, a mRNA used herein may be a modified mRNA, e.g., as described in U.S. Pat. No. 8,710,200, issued on Apr. 24, 2014, entitled “Engineered nucleic acids encoding a modified erythropoietin and their expression”. In some embodiments, a mRNA may comprise a 5′ methyl cap. In some embodiments, a mRNA may comprise a polyA tail, optionally of up to 160 nucleotides in length. A gene expression construct may encode a sequence of a protein that preferentially binds to a nucleic acid, e.g. a disease-associated repeat, or a protein, e.g. MBNL1, found in muscle cells. In some embodiments, the gene expression construct may be expressed, e.g., overexpressed, within the nucleus of a muscle cell. In some embodiments, the gene expression construct encodes a MBNL protein, e.g., MBNL1. In some embodiments, the gene expression constructs encodes a protein that comprises at least one zinc finger. In some embodiments, the gene expression construct encodes a protein that binds to a disease-associated repeat. In some embodiments, the gene expression construct encodes a protein that leads to a reduction in the expression of a disease-associated repeat. In some embodiments, the gene expression construct encodes a gene editing enzyme. Additional examples of nucleic acid constructs that may be used as molecular payloads are provided in International Patent Application Publication WO2017152149A1, published on Sep. 19, 2017, entitled, “Closed-Ended Linear Duplex Dna For Non-Viral Gene Transfer”; U.S. Pat. No. 8,853,377B2, issued on Oct. 7, 2014, entitled, “mRNA For Use In Treatment Of Human Genetic Diseases”; and US Patent U.S. Pat. No. 8,822,663B2, issued on Sep. 2, 2014, Engineered Nucleic Acids And Methods Of Use Thereof,” the contents of each of which are incorporated herein by reference in their entireties.

C. Linkers

Complexes described herein generally comprise a linker that connects a muscle-targeting agent to a molecular payload. A linker comprises at least one covalent bond. In some embodiments, a linker may be a single bond, e.g., a disulfide bond or disulfide bridge, that connects a muscle-targeting agent to a molecular payload. However, in some embodiments, a linker may connect a muscle-targeting agent to a molecular through multiple covalent bonds. In some embodiments, a linker may be a cleavable linker. However, in some embodiments, a linker may be a non-cleavable linker. A linker is generally stable in vitro and in vivo, and may be stable in certain cellular environments. Additionally, generally a linker does not negatively impact the functional properties of either the muscle-targeting agent or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A precursor to a linker typically will contain two different reactive species that allow for attachment to both the muscle-targeting agent and a molecular payload. In some embodiments, the two different reactive species may be a nucleophile and/or an electrophile. In some embodiments, a linker is connected to a muscle-targeting agent via conjugation to a lysine residue or a cysteine residue of the muscle-targeting agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle-targeting agent or thiol functionalized molecular payload via a 3-arylpropionitrile functional group. In some embodiments, a linker is connected to a muscle-targeting agent and/or a molecular payload via an amide bond, a hydrazide, a trizaole, a thioether, or a disulfide bond.

i. Cleavable Linkers

A cleavable linker may be a protease-sensitive linker, a pH-sensitive linker, or a glutathione-sensitive linker. These linkers are generally cleavable only intracellularly and are preferably stable in extracellular environments, e.g. extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include β-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by an disulfide exchange reaction with a glutathione species inside a cell. In some embodiments, the disulfide moiety further comprises at least one amino acid, e.g. a cysteine residue.

In some embodiments, the linker is a Val-cit linker (e.g., as described in U.S. Pat. No. 6,214,345, incorporated herein by reference). In some embodiments, before conjugation, the val-cit linker has a structure of:

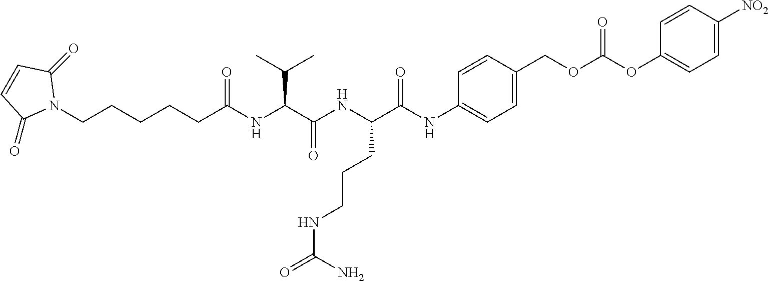 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, the val-cit linker has a structure of:

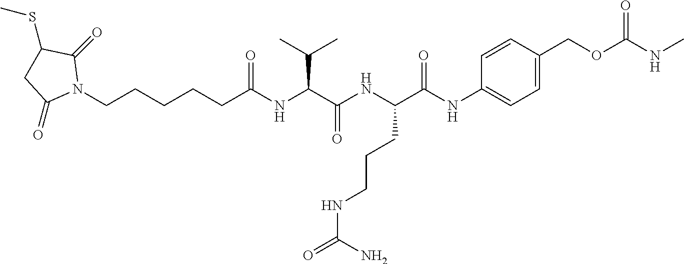

In some embodiments, before conjugation, the val-cit linker has a structure of:

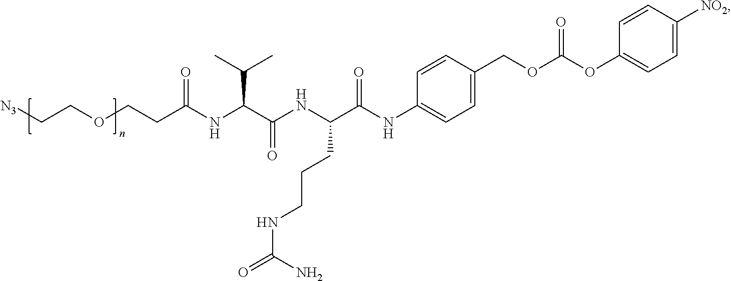

wherein n is any number from 0-10.

In some embodiments, after conjugation to an oligonucleotide, the val-cit linker has a structure of:

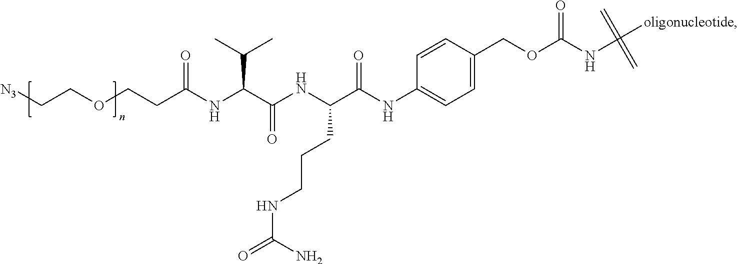

wherein n is any number from 0-10 (e.g., 3).

In some embodiments, after conjugation to an oligonucleotide, the val-cit linker has a structure of:

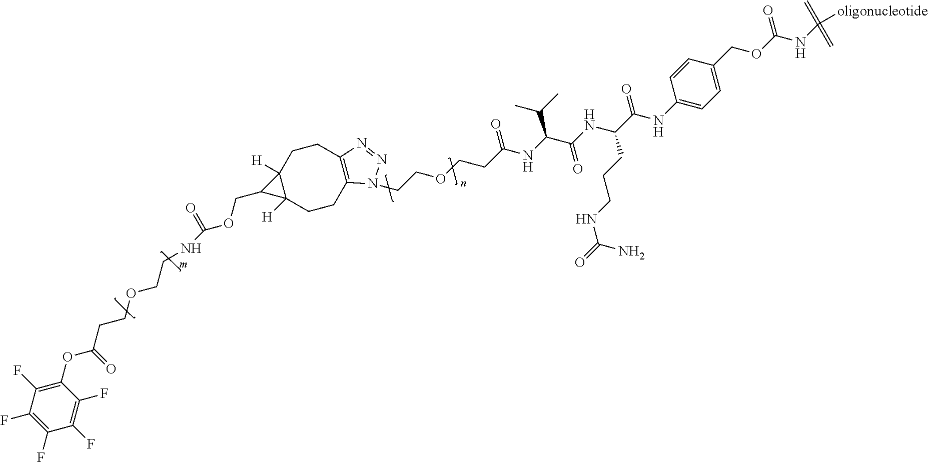

wherein n is any number from 0-10 (e.g., 3), and m is any number from 0-10 (e.g., 4).

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a molecular payload comprising a (G). sequence (see, e.g. Proft T. Sortase-mediated protein ligation: an emerging biotechnology tool for protein modification and immobilization. Biotechnol Lett. 2010, 32(1):1-10.). In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species 0, an optionally substituted sulfur species, or a poly(alkylene oxide), e.g. polyethylene oxide or polypropylene oxide.

iii. Linker conjugation

In some embodiments, a linker is connected to a muscle-targeting agent and/or molecular payload via a phosphate, thioether, ether, carbon-carbon, a carbamate, or amide bond. In some embodiments, a linker is connected to an oligonucleotide through a phosphate or phosphorothioate group, e.g. a terminal phosphate of an oligonucleotide backbone. In some embodiments, a linker is connected to an muscle-targeting agent, e.g. an antibody, through a lysine or cysteine residue present on the muscle-targeting agent

In some embodiments, a linker is connected to a muscle-targeting agent and/or molecular payload by a cycloaddition reaction between an azide and an alkyne to form a triazole, wherein the azide and the alkyne may be located on the muscle-targeting agent, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctane is as described in International Patent Application Publication WO2011136645, published on Nov. 3, 2011, entitled, “Fused Cyclooctyne Compounds And Their Use In Metal free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide and the alkyne may be located on the muscle-targeting agent, molecular payload, or the linker is as described in International Patent Application Publication WO2014065661, published on May 1, 2014, entitled, “Modified antibody, antibody-conjugate and process for the preparation thereof′; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker further comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is connected to a muscle-targeting agent and/or molecular payload by the Diels-Alder reaction between a dienophile and a diene/hetero-diene, wherein the dienophile and the diene/hetero-diene may be located on the muscle-targeting agent, molecular payload, or the linker. In some embodiments a linker is connected to a muscle-targeting agent and/or molecular payload by other pericyclic reactions, e.g. ene reaction. In some embodiments, a linker is connected to a muscle-targeting agent and/or molecular payload by an amide, thioamide, or sulfonamide bond reaction. In some embodiments, a linker is connected to a muscle-targeting agent and/or molecular payload by a condensation reaction to form an oxime, hydrazone, or semicarbazide group existing between the linker and the muscle-targeting agent and/or molecular payload.

In some embodiments, a linker is connected to a muscle-targeting agent and/or molecular payload by a conjugate addition reactions between a nucleophile, e.g. an amine or a hydroxyl group, and an electrophile, e.g. a carboxylic acid or an aldehyde. In some embodiments, a nucleophile may exist on a linker and an electrophile may exist on a muscle-targeting agent or molecular payload prior to a reaction between a linker and a muscle-targeting agent or molecular payload. In some embodiments, an electrophile may exist on a linker and a nucleophile may exist on a muscle-targeting agent or molecular payload prior to a reaction between a linker and a muscle-targeting agent or molecular payload. In some embodiments, an electrophile may be an azide, a pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

D. Examples of Antibody-Molecular Payload Complexes

Other aspects of the present disclosure provide complexes comprising any one the muscle targeting agent (e.g., a transferrin receptor antibodies) described herein covalently linked to any of the molecular payloads (e.g., an oligonucleotide) described herein. In some embodiments, the muscle targeting agent (e.g., a transferrin receptor antibody) is covalently linked to a molecular payload (e.g., an oligonucleotide) via a linker. Any of the linkers described herein may be used. In some embodiments, the linker is linked to the 5′ end, the 3′ end, or internally of the oligonucleotide. In some embodiments, the linker is linked to the antibody via a thiol-reactive linkage (e.g., via a cysteine in the antibody).

An exemplary structure of a complex comprising a transferrin receptor antibody covalently linked to an oligonucleotide via a Val-cit linker is provided below:

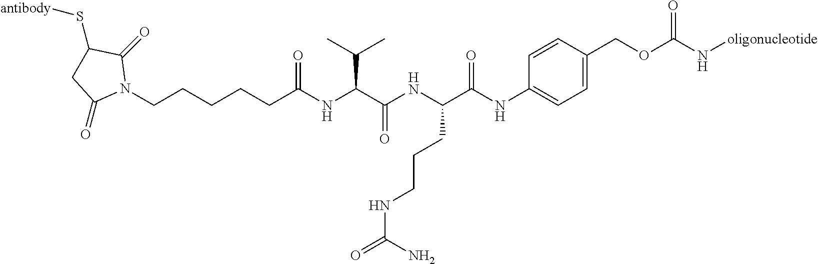

wherein the linker is linked to the 5′ end, the 3′ end, or internally of the oligonucleotide, and wherein the linker is linked to the antibody via a thiol-reactive linkage (e.g., via a cysteine in the antibody).

Another example of a structure of a complex comprising a transferrin receptor antibody covalently linked to a molecular payload via a Val-cit linker is provided below:

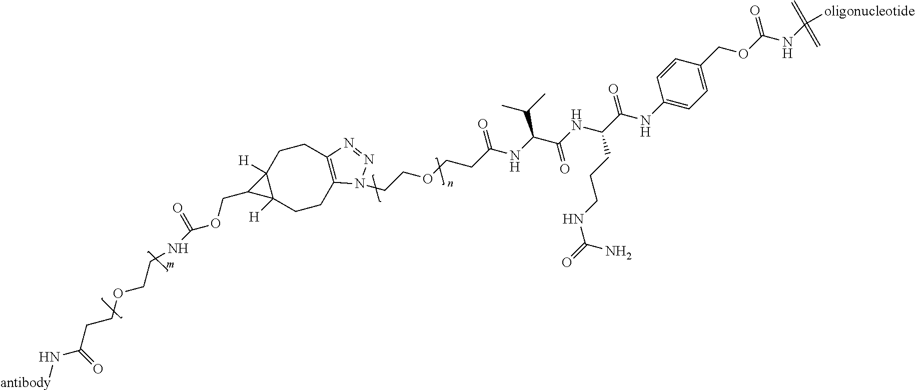

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4.

It should be appreciated that antibodies can be linked to oligonucleotides with different stoichiometries, a property that may be referred to as a drug to antibody ratios (DAR) with the “drug” being the oligonucleotide. In some embodiments, one oligonucleotide is linked to an antibody (DAR=1). In some embodiments, two oligonucleotides are linked to an antibody (DAR=2). In some embodiments, three oligonucleotides are linked to an antibody (DAR=3). In some embodiments, four oligonucleotides are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating oligonucleotides to different sites on an antibody and/or by conjugating multimers to one or more sites on antibody. For example, a DAR of 2 may be achieved by conjugating a single oligonucleotide to two different sites on an antibody or by conjugating a dimer oligonucleotide to a single site of an antibody.

In some embodiments, the complex described herein comprises a transferrin receptor antibody (e.g., an antibody or any variant thereof as described herein) covalently linked to an oligonucleotide targeting DMPK (e.g., an oligonucleotide having a region of complementarity to a DMPK gene sequence as set forth in SEQ ID NO: 15 or SEQ ID NO: 16). In some embodiments, the complex described herein comprises a transferrin receptor antibody (e.g., an antibody or any variant thereof as described herein) covalently linked to an oligonucleotide targeting DMPK (e.g., an oligonucleotide having a region of complementarity to a DMPK gene sequence as set forth in SEQ ID NO: 15 or SEQ ID NO: 16) via a linker (e.g., a Val-cit linker). In some embodiments, the linker (e.g., a Val-cit linker) is linked to the 5′ end, the 3′ end, or internally of the nucleotide targeting DMPK (e.g., an oligonucleotide having a region of complementarity to a DMPK gene sequence as set forth in SEQ ID NO: 15 or SEQ ID NO: 16). In some embodiments, the linker (e.g., a Val-cit linker) is linked to the antibody (e.g., an antibody or any variant thereof as described herein) via a thiol-reactive linkage (e.g., via a cysteine in the antibody).

In some embodiments, the complex described herein comprises a transferrin receptor antibody covalently linked to a DMPK targeting oligonucleotide, wherein the transferrin receptor antibody comprises a CDR-H1, a CDR-H2, and a CDR-H3 that are the same as the CDR-H1, CDR-H2, and CDR-H3 shown in Table 2; and a CDR-L1, a CDR-L2, and a CDR-L3 that are the same as the CDR-L1, CDR-L2, and CDR-L3 shown in Table 2.

In some embodiments, the complex described herein comprises a transferrin receptor antibody covalently linked to a DMPK targeting oligonucleotide, wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to a DMPK targeting oligonucleotide, wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to a DMPK targeting oligonucleotide, wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to a DMPK targeting oligonucleotide, wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to a DMPK targeting oligonucleotide via a linker (e.g., a Val-cit linker), wherein the transferrin receptor antibody comprises a CDR-H1, a CDR-H2, and a CDR-H3 that are the same as the CDR-H1, CDR-H2, and CDR-H3 shown in Table 2; and a CDR-L1, a CDR-L2, and a CDR-L3 that are the same as the CDR-L1, CDR-L2, and CDR-L3 shown in Table 2.

In some embodiments, the complex described herein comprises a transferrin receptor antibody covalently linked to a DMPK targeting oligonucleotide via a linker (e.g., a Val-cit linker), wherein the transferrin receptor antibody comprises a VH having the amino acid sequence of SEQ ID NO: 33 and a VL having the amino acid sequence of SEQ ID NO: 34. In some embodiments, the complex described herein comprises a transferrin receptor antibody covalently linked to a DMPK targeting oligonucleotide via a linker (e.g., a Val-cit linker), wherein the transferrin receptor antibody comprises a VH having the amino acid sequence of SEQ ID NO: 35 and a VL having the amino acid sequence of SEQ ID NO: 36.

In some embodiments, the complex described herein comprises a transferrin receptor antibody covalently linked to a DMPK targeting oligonucleotide via a linker (e.g., a Val-cit linker), wherein the transferrin receptor antibody comprises a heavy chain having the amino acid sequence of SEQ ID NO: 39 and a light chain having the amino acid sequence of SEQ ID NO: 40. In some embodiments, the complex described herein comprises a transferrin receptor antibody covalently linked to a DMPK targeting oligonucleotide via a linker (e.g., a Val-cit linker), wherein the transferrin receptor antibody comprises a heavy chain having the amino acid sequence of SEQ ID NO: 41 and a light chain having the amino acid sequence of SEQ ID NO: 42.

In some embodiments, the complex described herein comprises a transferrin receptor antibody covalently linked to a DMPK targeting oligonucleotide via a Val-cit linker, wherein the transferrin receptor antibody comprises a CDR-H1, a CDR-H2, and a CDR-H3 that are the same as the CDR-H1, CDR-H2, and CDR-H3 shown in Table 2; and a CDR-L1, a CDR-L2, and a CDR-L3 that are the same as the CDR-L1, CDR-L2, and CDR-L3 shown in Table 2, and wherein the complex comprises the structure of:

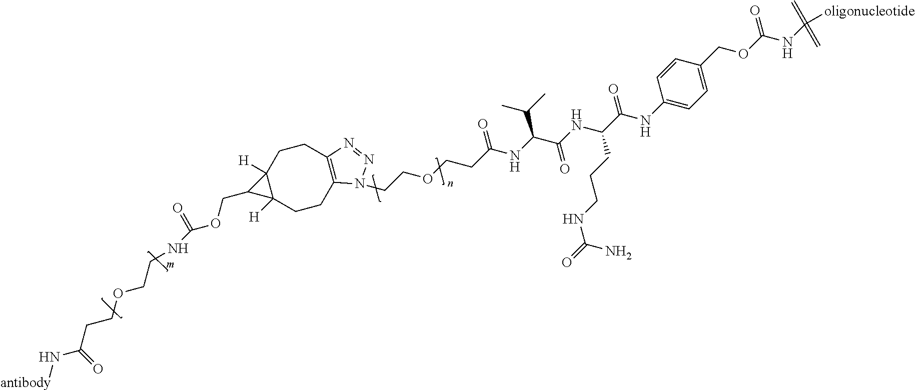

wherein n is 3 and m is 4, optionally wherein the DMPK targeting oligonucleotide (e.g., a gapmer) comprises at least 10, 11, 12, 13, 14, 15, 16, 17, 18, 19, or 20 consecutive nucleotides (e.g., 10, 11, 12, 13, 14, 15, 16, 17, 18, 19, or 20 consecutive nucleotides) of the nucleotide sequence of any one of SEQ ID NOs: 45-280.

In some embodiments, the complex described herein comprises a transferrin receptor antibody covalently linked to a DMPK targeting oligonucleotide via a Val-cit linker, wherein the transferrin receptor antibody comprises a VH having the amino acid sequence of SEQ ID NO: 33 and a VL having the amino acid sequence of SEQ ID NO: 34, and wherein the complex comprises the structure of:

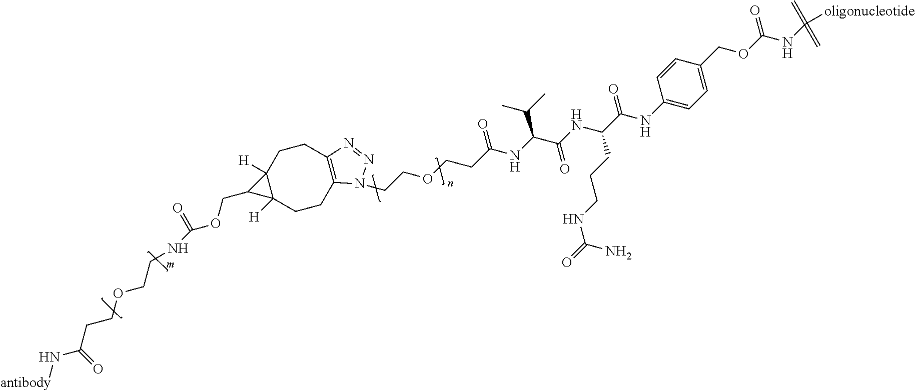

wherein n is 3 and m is 4, optionally wherein the DMPK targeting oligonucleotide (e.g., a gapmer) comprises at least 10, 11, 12, 13, 14, 15, 16, 17, 18, 19, or 20 consecutive nucleotides (e.g., 10, 11, 12, 13, 14, 15, 16, 17, 18, 19, or 20 consecutive nucleotides) of the nucleotide sequence of any one of SEQ ID NOs: 45-280.

In some embodiments, the complex described herein comprises a transferrin receptor antibody covalently linked to a DMPK targeting oligonucleotide via a Val-cit linker, wherein the transferrin receptor antibody comprises a VH having the amino acid sequence of SEQ ID NO: 35 and a VL having the amino acid sequence of SEQ ID NO: 36, and wherein the complex comprises the structure of:

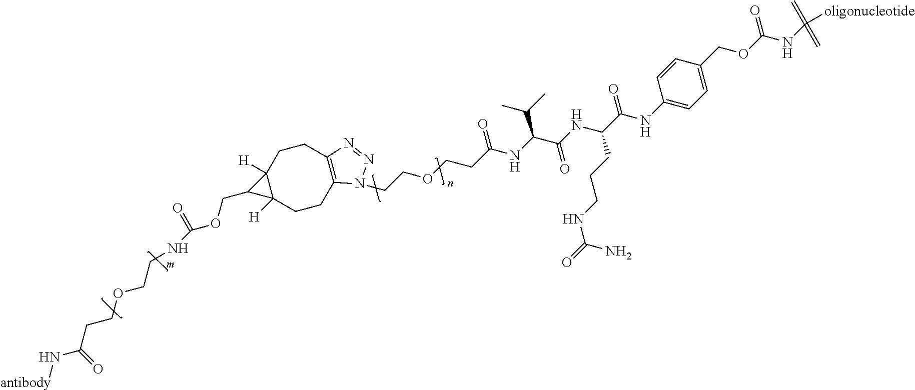

wherein n is 3 and m is 4, optionally wherein the DMPK targeting oligonucleotide (e.g., a gapmer) comprises at least 10, 11, 12, 13, 14, 15, 16, 17, 18, 19, or 20 consecutive nucleotides (e.g., 10, 11, 12, 13, 14, 15, 16, 17, 18, 19, or 20 consecutive nucleotides) of the nucleotide sequence of any one of SEQ ID NOs: 45-280.

In some embodiments, the complex described herein comprises a transferrin receptor antibody covalently linked to a DMPK targeting oligonucleotide via a Val-cit linker, wherein the transferrin receptor antibody comprises a heavy chain having the amino acid sequence of SEQ ID NO: 39 and a light chain having the amino acid sequence of SEQ ID NO: 40, and wherein the complex comprises the structure of:

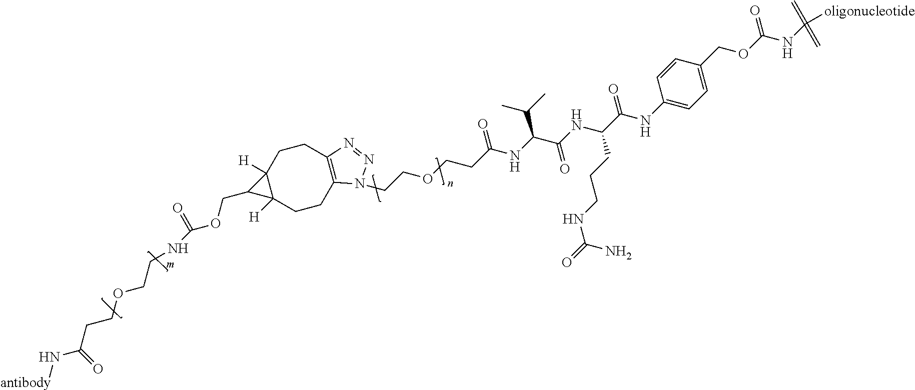

wherein n is 3 and m is 4, optionally wherein the DMPK targeting oligonucleotide (e.g., a gapmer) comprises at least 10, 11, 12, 13, 14, 15, 16, 17, 18, 19, or 20 consecutive nucleotides (e.g., 10, 11, 12, 13, 14, 15, 16, 17, 18, 19, or 20 consecutive nucleotides) of the nucleotide sequence of any one of SEQ ID NOs: 45-280.

In some embodiments, the complex described herein comprises a transferrin receptor antibody covalently linked to a DMPK targeting oligonucleotide via a Val-cit linker, wherein the transferrin receptor antibody comprises a heavy chain having the amino acid sequence of SEQ ID NO: 41 and a light chain having the amino acid sequence of SEQ ID NO: 42, and wherein the complex comprises the structure of:

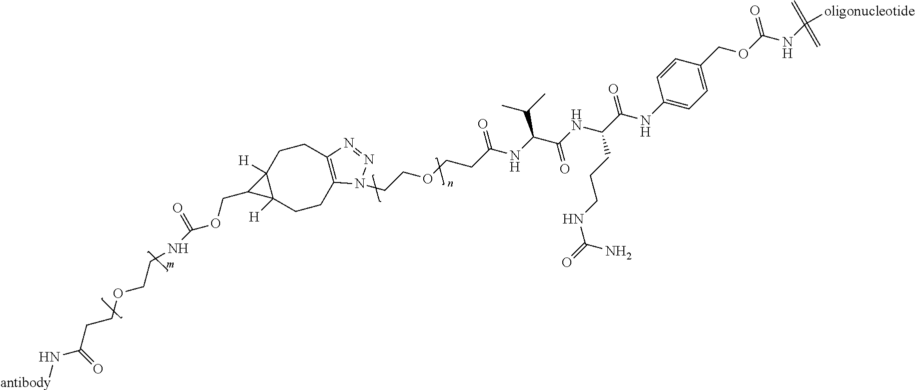

wherein n is 3 and m is 4, optionally wherein the DMPK targeting oligonucleotide (e.g., a gapmer) comprises at least 10, 11, 12, 13, 14, 15, 16, 17, 18, 19, or 20 consecutive nucleotides (e.g., 10, 11, 12, 13, 14, 15, 16, 17, 18, 19, or 20 consecutive nucleotides) of the nucleotide sequence of any one of SEQ ID NOs: 45-280.

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the a complexes in a required amount in a selected solvent with one or a combination of ingredients enumerated above, as required, followed by filtered sterilization.

In some embodiments, a composition may contain at least about 0.1% of the a complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently to a molecular payload as described herein are effective in treating myotonic dystrophy. In some embodiments, complexes are effective in treating myotonic dystrophy type 1 (DM1). In some embodiments, DM1 is associated with an expansion of a CTG trinucleotide repeat in the 3′ non-coding region of DMPK. In some embodiments, the nucleotide expansions lead to toxic RNA repeats capable of forming hairpin structures that bind critical intracellular proteins, e.g., muscleblind-like proteins, with high affinity.

In some embodiments, a subject may be a human subject, a non-human primate subject, a rodent subject, or any suitable mammalian subject. In some embodiments, a subject may have myotonic dystrophy. In some embodiments, a subject has a DMPK allele, which may optionally contain a disease-associated repeat. In some embodiments, a subject may have a DMPK allele with an expanded disease-associated-repeat that comprises about 2-10 repeat units, about 2-50 repeat units, about 2-100 repeat units, about 50-1,000 repeat units, about 50-500 repeat units, about 50-250 repeat units, about 50-100 repeat units, about 500-10,000 repeat units, about 500-5,000 repeat units, about 500-2,500 repeat units, about 500-1,000 repeat units, or about 1,000-10,000 repeat units. In some embodiments, a subject is suffering from symptoms of DM1, e.g. muscle atrophy or muscle loss. In some embodiments, a subject is not suffering from symptoms of DM1. In some embodiments, subjects have congenital myotonic dystrophy.

An aspect of the disclosure includes a methods involving administering to a subject an effective amount of a complex as described herein. In some embodiments, an effective amount of a pharmaceutical composition that comprises a complex comprising a muscle-targeting agent covalently to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently to a molecular payload is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g. safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment.

In some embodiments, an initial candidate dosage is about 1-50, 1-25, 1-10, 1-5, 5-100, 5-50, 5-25, 5-10, 10-100, 10-75, 10-50, 10-25, 10-20, 25-100, 25-75, or 25-50 mg/kg. In some embodiments, an initial candidate dosage is about 1-20, 1-15, 1-10, 1-5, 1-3, 1-2, 5-20, 5-15, or 5-10 mg/kg. In some embodiments, an initial candidate dosage is about 1, 2, 3, 4, 5, 6, 7, 8, 9, 10, 15, or 20 mg/kg. In some embodiments, the dosage referred to herein is the dose of the molecular payload (e.g., oligonucleotide).

The efficacy of treatment may be assessed using any suitable methods. In some embodiments, the efficacy of treatment may be assessed by evaluation of observation of symptoms associated with DM1, e.g. muscle atrophy or muscle weakness, through measures of a subject's self-reported outcomes, e.g. mobility, self-care, usual activities, pain/discomfort, and anxiety/depression, or by quality-of-life indicators, e.g. lifespan.

In some embodiments, a pharmaceutical composition that comprises a complex comprising a muscle-targeting agent covalently to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a single dose or administration of a pharmaceutical composition that comprises a complex comprising a muscle-targeting agent covalently to a molecular payload described herein to a subject is sufficient to inhibit activity or expression of a target gene for at least 1-5, 1-10, 5-15, 10-20, 15-30, 20-40, 25-50, or more days. In some embodiments, a single dose or administration of a pharmaceutical composition that comprises a complex comprising a muscle-targeting agent covalently to a molecular payload described herein to a subject is sufficient to inhibit activity or expression of a target gene for at least 1, 2, 3, 4, 5, 6, 7, 8, 9, 10, 11, 12, 15, 20, or 24 weeks. In some embodiments, a single dose or administration of a pharmaceutical composition that comprises a complex comprising a muscle-targeting agent covalently to a molecular payload described herein to a subject is sufficient to inhibit activity or expression of a target gene for at least 1-5, 1-10, 2-5, 2-10, 4-8, 4-12, 5-10, 5-12, 5-15, 8-12, 8-15, 10-12, 10-15, 10-20, 12-15, 12-20, 15-20, or 15-25 weeks. In some embodiments, a single dose or administration of a pharmaceutical composition that comprises a complex comprising a muscle-targeting agent covalently to a molecular payload described herein to a subject is sufficient to inhibit activity or expression of a target gene for at least 1, 2, 3, 4, 5, or 6 months.

In some embodiments, a single dose or administration of a pharmaceutical composition that comprises a complex comprising a muscle-targeting agent covalently to a molecular payload described herein to a subject persists or remains in the subject for at least 1-5, 1-10, 5-15, 10-20, 15-30, 20-40, 25-50, or more days. In some embodiments, a single dose or administration of a pharmaceutical composition that comprises a complex comprising a muscle-targeting agent covalently to a molecular payload described herein to a subject persists or remains in the subject for at least 1, 2, 3, 4, 5, 6, 7, 8, 9, 10, 11, 12, 15, 20, or 24 weeks. In some embodiments, a single dose or administration of a pharmaceutical composition that comprises a complex comprising a muscle-targeting agent covalently to a molecular payload described herein to a subject persists or remains in the subject for at least 1-5, 1-10, 2-5, 2-10, 4-8, 4-12, 5-10, 5-12, 5-15, 8-12, 8-15, 10-12, 10-15, 10-20, 12-15, 12-20, 15-20, or 15-25 weeks. In some embodiments, a single dose or administration of a pharmaceutical composition that comprises a complex comprising a muscle-targeting agent covalently to a molecular payload described herein to a subject persists or remains in the subject for at least 1, 2, 3, 4, 5, or 6 months.

In some embodiments, multiple doses or administrations of a pharmaceutical composition that comprises a complex comprising a muscle-targeting agent covalently to a molecular payload described herein are delivered to a subject. In some embodiments, multiple doses of a pharmaceutical composition comprise delivering 2, 3, 4, 5, 6, 7, 8, 9, or 10 doses to a subject. In some embodiments, multiple doses of a pharmaceutical composition comprise delivering a dose to a subject every 1, 2, 3, 4, 5, 6, 7, 8, 9, 10, 11, 12, 13, 14, 15, or 16 weeks. In some embodiments, multiple doses of a pharmaceutical composition comprise delivering a dose to a subject once every 4 weeks. In some embodiments, multiple doses of a pharmaceutical composition comprise delivering a dose to a subject once every 1-10, 2-5, 2-10, 4-8, 4-12, 5-10, 5-12, 5-15, 8-12, 8-16, 10-12, 10-15, 10-20, 12-15, 12-20, 15-20, or 15-25 weeks. In some embodiments, multiple doses of a pharmaceutical composition comprise delivering a dose to a subject on a biweekly (i.e., every two weeks), bimonthly (i.e., every two months), or quarterly schedule (i.e., every twelve weeks).

In some embodiments, a single dose or administration is about 1-50, 1-25, 1-10, 1-15, 1-5, 5-100, 5-50, 5-25, 5-10, 10-100, 10-75, 10-50, 10-25, 10-20, 25-100, 25-75, or 25-50 mg/kg. In some embodiments, a single dose or administration is about 1-20, 1-15, 1-10, 1-5, 1-3, 1-2, 5-20, 5-15, or 5-10 mg/kg. In some embodiments, a single dose or administration is about 1, 2, 3, 4, 5, 6, 7, 8, 9, 10, 11, 12, 13, 14, 15, or 20 mg/kg. In some embodiments, the dosage referred to herein is the dose of the molecular payload (e.g., oligonucleotide).

In some embodiments, a pharmaceutical composition that comprises a complex comprising a muscle-targeting agent covalently to a molecular payload described herein is delivered to a subject every 1, 2, 3, 4, 5, 6, 7, 8, 9, 10, 11, 12, 13, 14, 15, or 16 weeks. In some embodiments, a pharmaceutical composition that comprises a complex comprising a muscle-targeting agent covalently to a molecular payload described herein is delivered to a subject every 1-10, 2-5, 2-10, 4-8, 4-12, 5-10, 5-12, 5-15, 8-12, 8-16, 9-15, 10-12, 10-14, 10-15, 10-20, 11-13, 11-15, 12-15, 12-16, 12-20, 15-20, or 15-25 weeks. In some embodiments, a pharmaceutical composition that comprises a complex comprising a muscle-targeting agent covalently to a molecular payload described herein is delivered to a subject on a biweekly (i.e., every two weeks), bimonthly (i.e., every two months), or quarterly schedule (i.e., every twelve weeks).

In some embodiments, a pharmaceutical composition that comprises a complex comprising a muscle-targeting agent covalently to a molecular payload described herein at a molecular payload (e.g., oligonucleotide) dose of 1-15 mg/kg is delivered to a subject every 1, 2, 3, 4, 5, 6, 7, 8, 9, 10, 11, 12, 13, 14, 15, or 16 weeks. In some embodiments, a pharmaceutical composition that comprises a complex comprising a muscle-targeting agent covalently to a molecular payload described herein at a molecular payload (e.g., oligonucleotide) dose 1-15 mg/kg is delivered to a subject every 1-10, 2-5, 2-10, 4-8, 4-12, 5-10, 5-12, 5-15, 8-12, 8-16, 9-15, 10-12, 10-14, 10-15, 10-20, 11-13, 11-15, 12-15, 12-16, 12-20, 15-20, or 15-25 weeks. In some embodiments, a pharmaceutical composition that comprises a complex comprising a muscle-targeting agent covalently to a molecular payload described herein at a molecular payload (e.g., oligonucleotide) dose of 1-15 mg/kg is delivered to a subject on a biweekly (i.e., every two weeks), bimonthly (i.e., every two months), or quarterly schedule (i.e., every twelve weeks).

In some embodiments, a pharmaceutical composition may comprises more than one complex comprising a muscle-targeting agent covalently to a molecular payload. In some embodiments, a pharmaceutical composition may further comprise any other suitable therapeutic agent for treatment of a subject, e.g. a human subject having DM1. In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Targeting Dmpk with Transfected Antisense Oligonucleotides

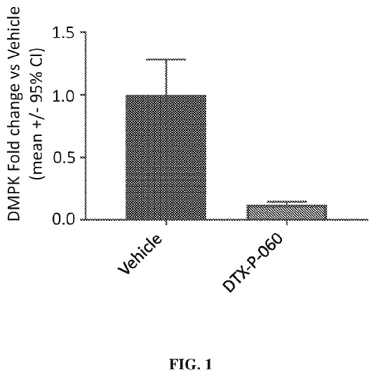

A gapmer antisense oligonucleotide that targets both wild-type and mutant alleles of DMPK (DTX-P-060) was tested in vitro for its ability to reduce expression levels of DMPK in an immortalized cell line. Briefly, Hepa 1-6 cells were transfected with the DTX-P-060 (100 nM) formulated with lipofectamine 2000. DMPK expression levels were evaluated 72 hours following transfection. A control experiment was also performed in which vehicle (phosphate-buffered saline) was delivered to Hepa 1-6 cells in culture and the cells were maintained for 72 hours. As shown in FIG. 1, it was found that the DTX-P-060 reduced DMPK expression levels by ˜90% compared with controls.

### Example 2: Targeting DMPK with a Muscle-Targeting Complex

A muscle-targeting complex was generated comprising the DMPK ASO used in Example 1 (DTX-P-060) covalently linked, via a cathepsin cleavable linker, to DTX-A-002 (RI7 217 (Fab)), an anti-transferrin receptor antibody.

Briefly, a maleimidocaproyl-L-valine-L-citrulline-p-aminobenzyl alcohol p-nitrophenyl carbonate (MC-Val-Cit-PABC-PNP) linker molecule was coupled to NH₂-C₆-DTX-P-060 using an amide coupling reaction. Excess linker and organic solvents were removed by gel permeation chromatography. The purified Val-Cit-linker-DTX-P-060 was then coupled to a thiol-reactive anti-transferrin receptor antibody (DTX-A-002).

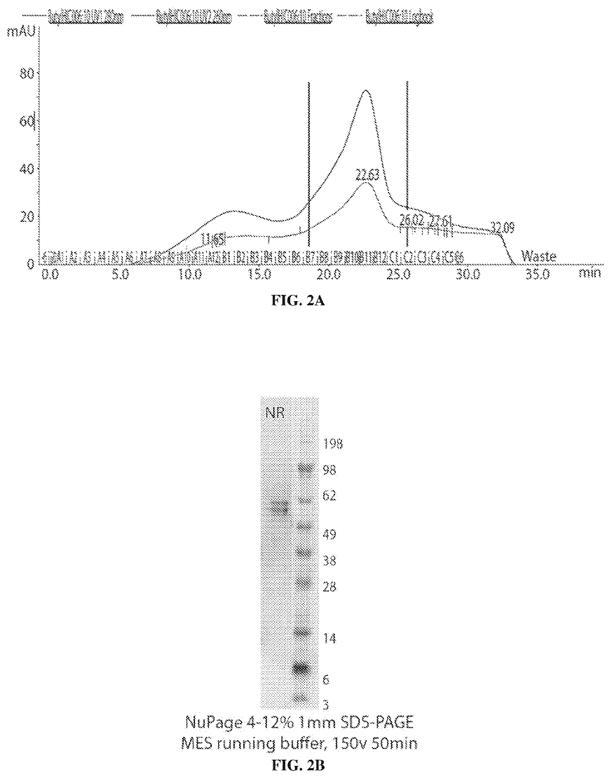

The product of the antibody coupling reaction was then subjected to hydrophobic interaction chromatography (HIC-HPLC). FIG. 2A shows a resulting HIC-HPLC chromatogram, in which fractions B7-C2 of the chromatogram (denoted by vertical lines) contained antibody-oligonucleotide complexes (referred to as DTX-C-008) comprising one or two DMPK ASO molecules covalently attached to DTX-A-002, as determined by SDS-PAGE. These HIC-HPLC fractions were combined and densitometry confirmed that this sample of DTX-C-008 complexes had an average ASO to antibody ratio of 1.48. SDS-PAGE analysis demonstrated that 86.4% of this sample of DTX-C-008 complexes comprised DTX-A-002 linked to either one or two DMPK ASO molecules (FIG. 2B).

Using the same methods as described above, a control complex was generated comprising the DMPK ASO used in Example 1 (DTX-P-060) covalently linked via a Val-Cit linker to an IgG2a (Fab) antibody (DTX-C-007).

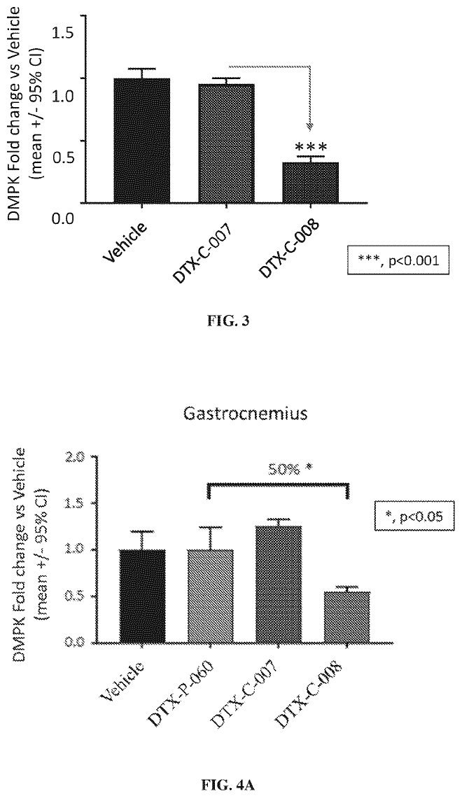

The purified DTX-C-008 was then tested for cellular internalization and inhibition of DMPK. Hepa 1-6 cells, which have relatively high expression levels of transferrin receptor, were incubated in the presence of vehicle control, DTX-C-008 (100 nM), or DTX-C-007 (100 nM) for 72 hours. After the 72 hour incubation, the cells were isolated and assayed for expression levels of DMPK (FIG. 3). Cells treated with the DTX-C-008 demonstrated a reduction in DMPK expression by ˜65% relative to the cells treated with the vehicle control. Meanwhile, cells treated with the DTX-C-007 had DMPK expression levels comparable to the vehicle control (no reduction in DMPK expression). These data indicate that the anti-transferrin receptor antibody of the DTX-C-008 enabled cellular internalization of the complex, thereby allowing the DMPK ASO to inhibit expression of DMPK.

### Example 3: Targeting DMPK in Mouse Muscle Tissues with a Muscle-Targeting Complex

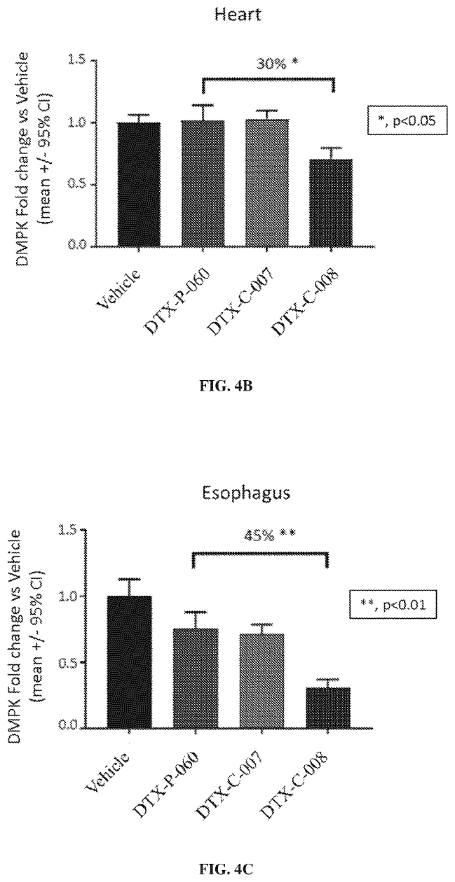

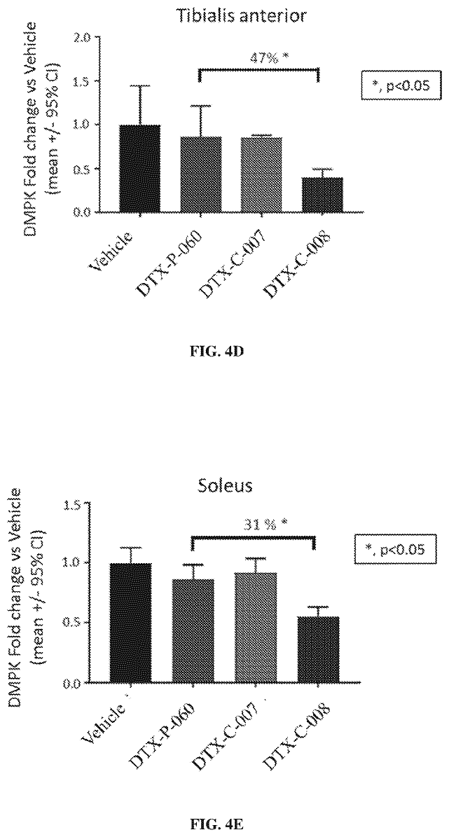

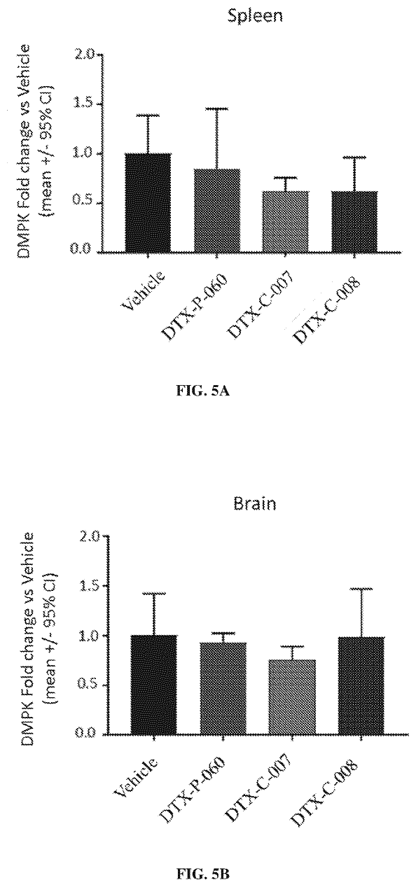

The muscle-targeting complex described in Example 2, DTX-C-008, was tested for inhibition of DMPK in mouse tissues. C57BL/6 wild-type mice were intravenously injected with a single dose of a vehicle control, DTX-P-060 (3 mg/kg of RNA), DTX-C-008 (3 mg/kg of RNA, corresponding to 20 mg/kg antibody conjugate), or DTX-C-007 (3 mg/kg of RNA, corresponding to 20 mg/kg antibody conjugate). DTX-P-060, the DMPK ASO as described in Example 1, was used as a control. Each experimental condition was replicated in three individual C57BL/6 wild-type mice. Following a seven-day period after injection, the mice were euthanized and segmented into isolated tissue types. Individual tissue samples were subsequently assayed for expression levels of DMPK (FIGS. 4A-4E and 5A-5B).

Mice treated with the DTX-C-008 complex demonstrated a reduction in DMPK expression in a variety of skeletal, cardiac, and smooth muscle tissues. For example, as shown in FIGS. 4A-4E, DMPK expression levels were significantly reduced in gastrocnemius (50% reduction), heart (30% reduction), esophagus (45% reduction), tibialis anterior (47% reduction), and soleus (31% reduction) tissues, relative to the mice treated with the vehicle control. Meanwhile, mice treated with the DTX-C-007 complex had DMPK expression levels comparable to the vehicle control (no reduction in DMPK expression) for all assayed muscle tissue types.

Mice treated with the DTX-C-008 complex demonstrated no change in DMPK expression in non-muscle tissues such as spleen and brain tissues (FIGS. 5A and 5B). These data indicate that the anti-transferrin receptor antibody of the DTX-C-008 enabled cellular internalization of the complex into muscle-specific tissues in an in vivo mouse model, thereby allowing the DMPK ASO to inhibit expression of DMPK. These data further demonstrate that the DTX-C-008 complex is capable of specifically targeting muscle tissues.

### Example 4: Targeting DMPK in Mouse Muscle Tissues with a Muscle-Targeting Complex

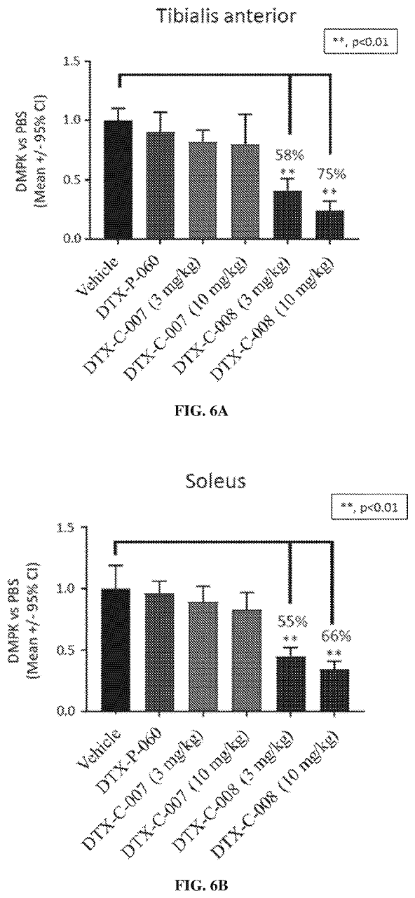

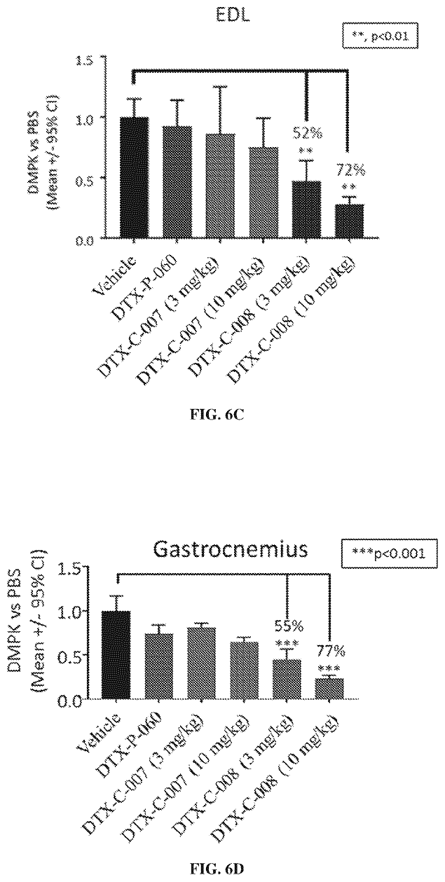

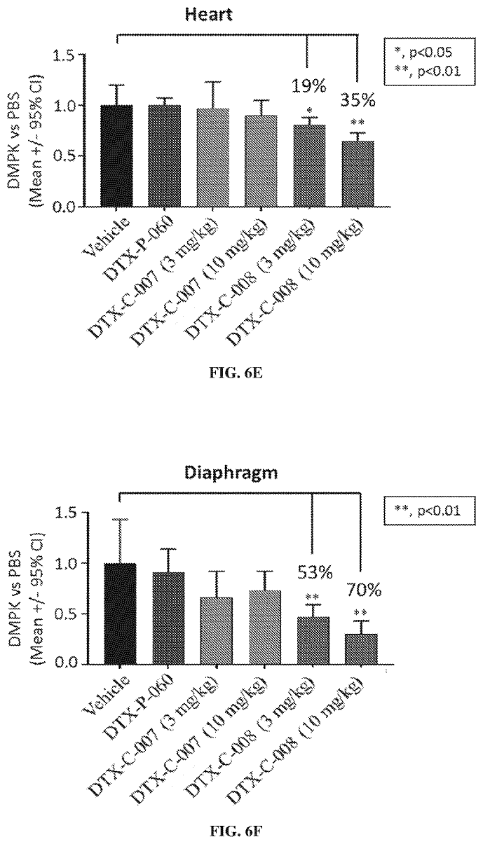

The muscle-targeting complex described in Example 2, DTX-C-008, was tested for dose-dependent inhibition of DMPK in mouse tissues. C57BL/6 wild-type mice were intravenously injected with a single dose of a vehicle control (phosphate-buffered saline, PBS), DTX-P-060 (10 mg/kg of RNA), DTX-C-008 (3 mg/kg or 10 mg/kg of RNA, wherein 3 mg/kg corresponds to 20 mg/kg antibody conjugate), or DTX-C-007 (3 mg/kg or 10 mg/kg of RNA, wherein 3 mg/kg corresponds to 20 mg/kg antibody conjugate). DTX-P-060, the DMPK ASO as described in Example 1, was used as a control. Each experimental condition was replicated in five individual C57BL/6 wild-type mice. Following a seven-day period after injection, the mice were euthanized and segmented into isolated tissue types. Individual tissue samples were subsequently assayed for expression levels of DMPK (FIGS. 6A-6F).

Mice treated with the DTX-C-008 complex demonstrated a reduction in DMPK expression in a variety of skeletal muscle tissues. As shown in FIGS. 6A-6F, DMPK expression levels were significantly reduced in tibialis anterior (58% and 75% reduction for 3 mg/kg and 10 mg/kg DTX-C-008, respectively), soleus (55% and 66% reduction for 3 mg/kg and 10 mg/kg DTX-C-008, respectively), extensor digitorum longus (EDL) (52% and 72% reduction for 3 mg/kg and 10 mg/kg DTX-C-008, respectively), gastrocnemius (55% and 77% reduction for 3 mg/kg and 10 mg/kg DTX-C-008, respectively), heart (19% and 35% reduction for 3 mg/kg and 10 mg/kg DTX-C-008, respectively), and diaphragm (53% and 70% reduction for 3 mg/kg and 10 mg/kg DTX-C-008, respectively) tissues, relative to the mice treated with the vehicle control. Notably, all assayed muscle tissue types experienced dose-dependent inhibition of DMPK, with greater reduction in DMPK levels at 10 mg/kg antibody conjugate relative to 3 mg/kg antibody conjugate.

Meanwhile, mice treated with the control DTX-C-007 complex had DMPK expression levels comparable to the vehicle control (no reduction in DMPK expression) for all assayed muscle tissue types. These data indicate that the anti-transferrin receptor antibody of the DTX-C-008 enabled cellular internalization of the complex into muscle-specific tissues in an in vivo mouse model, thereby allowing the DMPK ASO to inhibit expression of DMPK. These data further demonstrate that the DTX-C-008 complex is capable of specifically targeting muscle tissues for dose-dependent inhibition of DMPK.

### Example 5: Targeting DMPK in Cynomolgus Monkey Muscle Tissues with a Muscle-Targeting Complex

A muscle-targeting complex comprising DTX-P-060 (DTX-C-012), was generated and purified using methods described in Example 2. DTX-C-012 is a complex comprising a human anti-transferrin antibody covalently linked, via a cathepsin cleavable Val-Cit linker, to DTX-P-060, an antisense oligonucleotide that targets DMPK. Following HIC-HPLC purification, densitometry confirmed that DTX-C-012 had an average ASO to antibody ratio of 1.32, and SDS-PAGE revealed a purity of 92.3%.

DTX-C-012 was tested for dose-dependent inhibition of DMPK in male cynomolgus monkey tissues. Male cynomolgus monkeys (19-31 months; 2-3 kg) were intravenously injected with a single dose of a saline control, DTX-P-060 (naked DMPK ASO) (10 mg/kg of RNA), or DTX-C-012 (10 mg/kg of RNA) on Day 0. Each experimental condition was replicated in three individual male cynomolgus monkeys. On Day 7 after injection, tissue biopsies (including muscle tissues) were collected. DMPK mRNA expression levels, ASO detection assays, serum clinical chemistries, tissue histology, clinical observations, and body weights were analyzed. The monkeys were euthanized on Day 14.

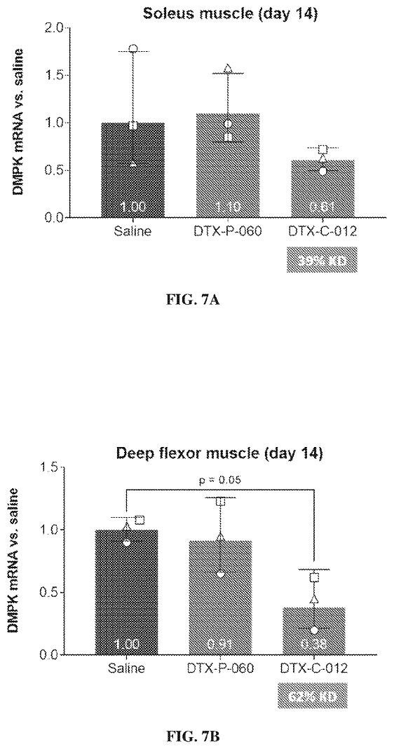

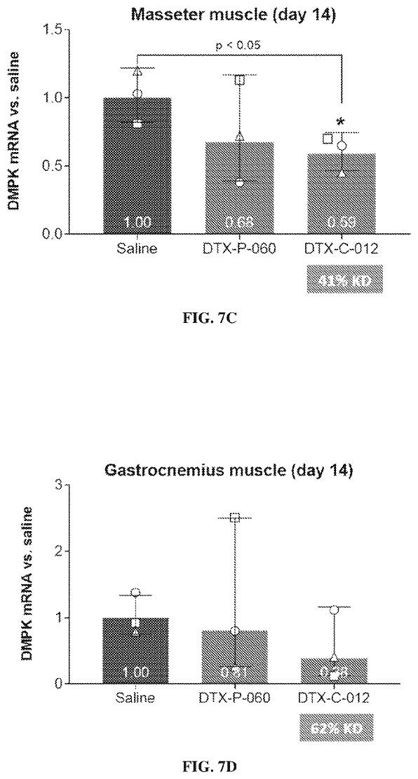

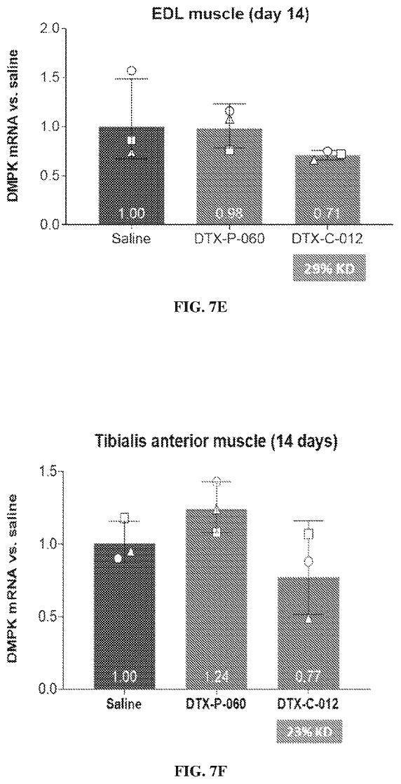

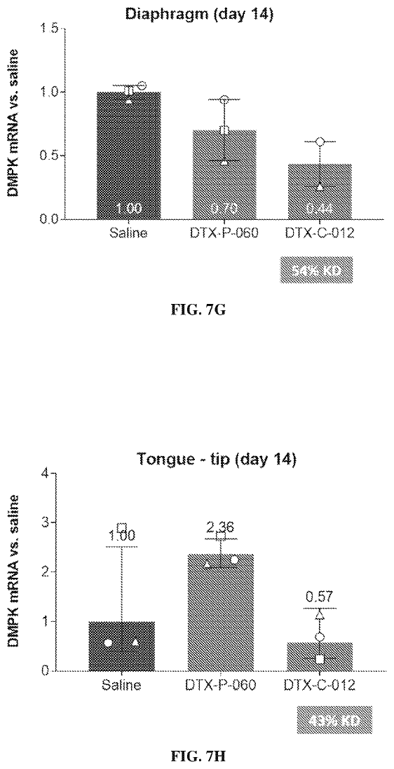

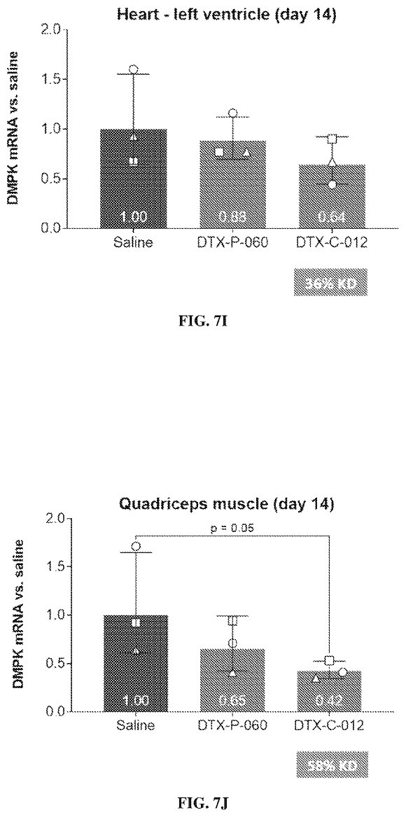

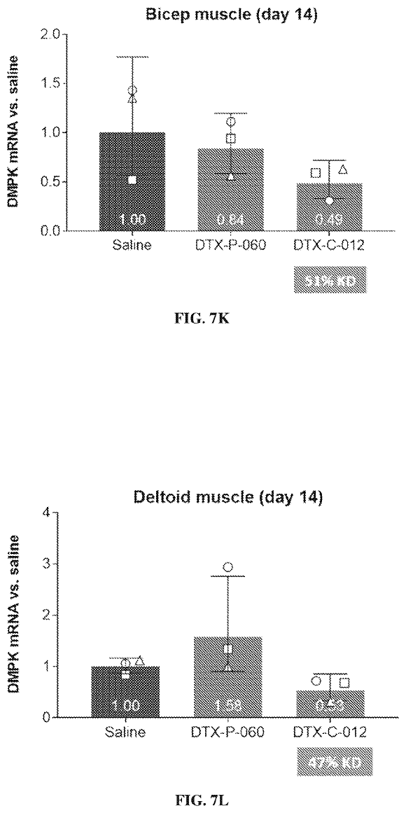

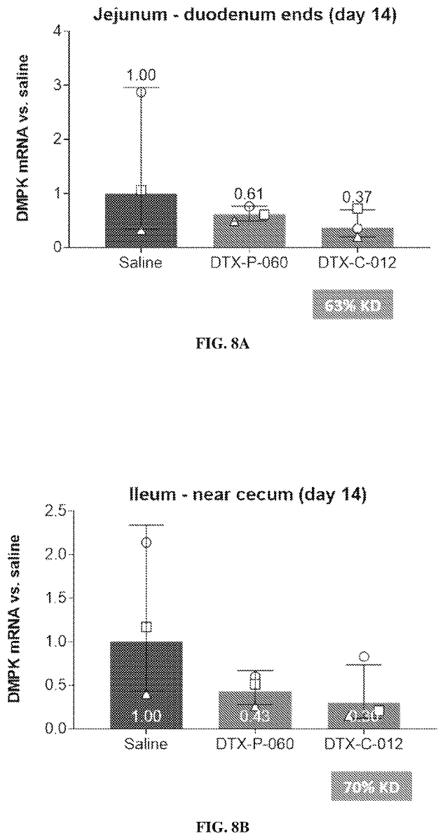

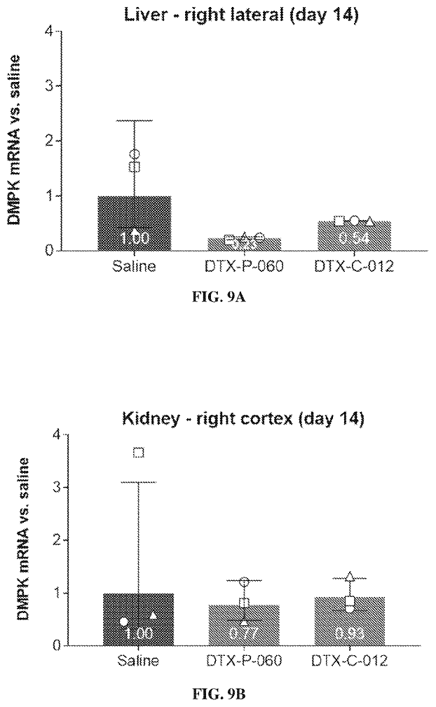

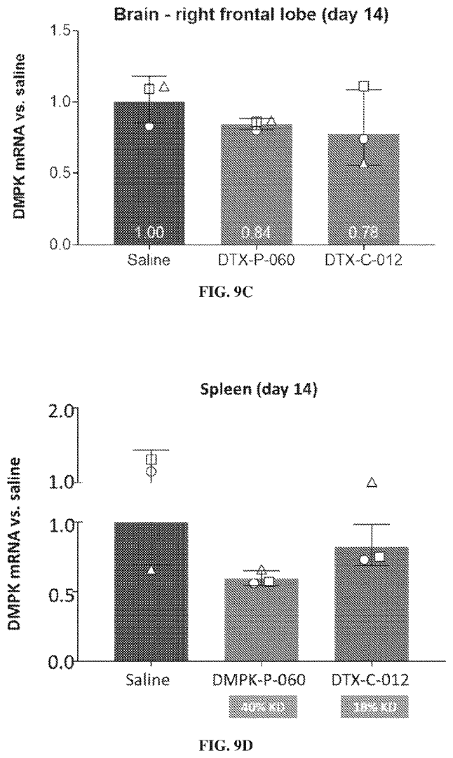

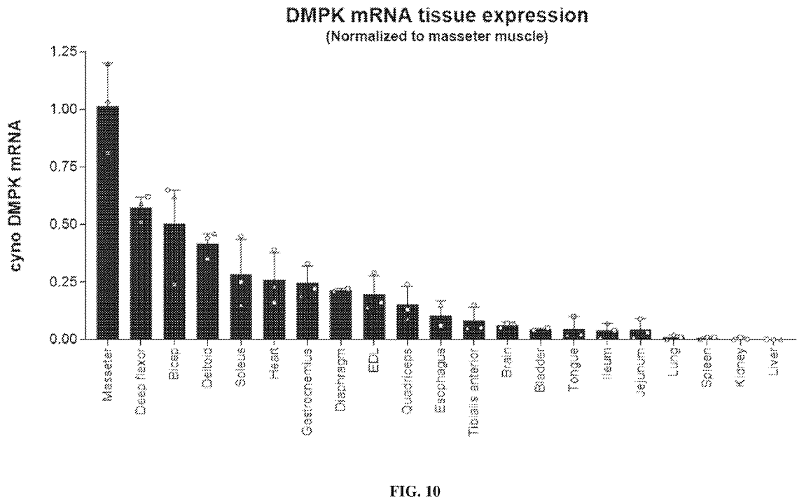

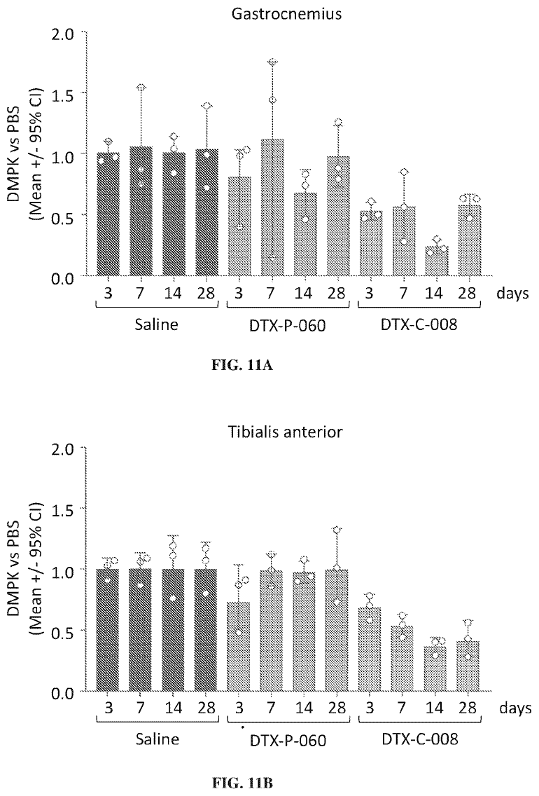

Significant knockdown (KD) of DMPK mRNA expression using DTX-C-012 was observed in soleus, deep flexor, and masseter muscles relative to saline control, with 39% KD, 62% KD, and 41% KD, respectively (FIGS. 7A-7C). Robust knockdown of DMPK mRNA expression DTX-C-012 was further observed in gastrocnemius (62% KD; FIG. 7D), EDL (29% KD; FIG. 7E), tibialis anterior muscle (23% KD; FIG. 7F), diaphragm (54% KD; FIG. 7G), tongue (43% KD; FIG. 7H), heart muscle (36% KD; FIG. 7I), quadriceps (58% KD; FIG. 7J), bicep (51% KD; FIG. 7K), and deltoid muscles (47% KD; FIG. 7L). Knockdown of DMPK mRNA expression DTX-C-012 in smooth muscle was also observed in the intestine, with 63% KD at jejunum-duodenum ends (FIG. 8A) and 70% KD in ileum (FIG. 8B). Notably, naked DMPK ASO (i.e., not linked to a muscle-targeting agent), DTX-P-060, had minimal effects on DMPK expression levels relative to the vehicle control (i.e., little or no reduction in DMPK expression) for all assayed muscle tissue types. Monkeys treated with the DTX-C-012 complex demonstrated no change in DMPK expression in non-muscle tissues, such as liver, kidney, brain, and spleen tissues (FIGS. 9A-9D). Additional tissues were examined, as depicted in FIG. 10, which shows normalized DMPK mRNA tissue expression levels across several tissue types in cynomolgus monkeys. (N=3 male cynomolgus monkeys)

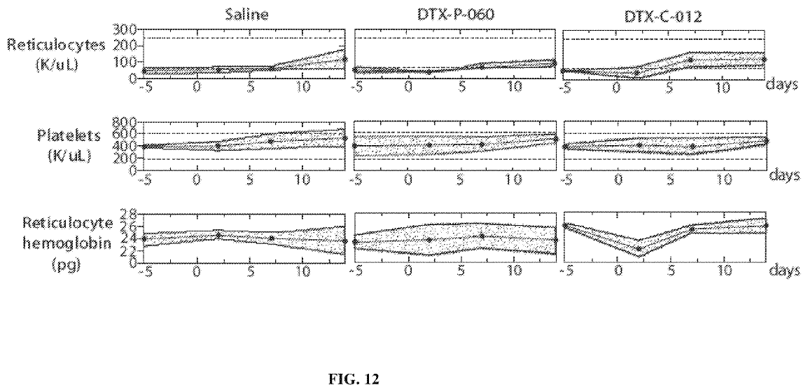

Prior to euthanization, all monkeys were tested for reticulocyte levels, platelet levels, hemoglobin expression, alanine aminotransferase (ALT) expression, aspartate aminotransferase (AST) expression, and blood urea nitrogen (BUN) levels on days 2, 7, and 14 after dosing. As shown in FIG. 12, monkeys dosed with antibody-oligonucleotide complex had normal reticulocyte levels, platelet levels, hemoglobin expression, alanine aminotransferase (ALT) expression, aspartate aminotransferase (AST) expression, and blood urea nitrogen (BUN) levels throughout the length of the experiment. These data show that a single dose of a complex comprising DTX-P-060 is safe and tolerated in cynomolgus monkeys.

These data demonstrate that the anti-transferrin receptor antibody of the DTX-C-012 complex enabled cellular internalization of the complex into muscle-specific tissues in an in vivo cynomolgus monkey model, thereby allowing the DMPK ASO (DTX-P-060) to inhibit expression of DMPK. These data further demonstrate that the DTX-C-012 complex is capable of specifically targeting muscle tissues for dose-dependent inhibition of DMPK without substantially impacting non-muscle tissues. This is direct contrast with the limited ability of DTX-P-060, a naked DMPK ASO (not linked to a muscle-targeting agent), to inhibit expression of DMPK in muscle tissues of an in vivo cynomolgus monkey model.

### Example 6: Targeting DMPK in Mouse Muscle Tissues with a Muscle-Targeting Complex

The muscle-targeting complex described in Example 2, DTX-C-008, was tested for time-dependent inhibition of DMPK in mouse tissues. C57BL/6 wild-type mice were intravenously injected with a single dose of a vehicle control (saline), DTX-P-060 (10 mg/kg of RNA), or DTX-C-008 (10 mg/kg of RNA) and euthanized after a prescribed period of time, as described in Table 3. Following euthanization, the mice were segmented into isolated tissue types and tissue samples were subsequently assayed for expression levels of DMPK (FIGS. 11A-11B).

TABLE 3
Experimental conditions

| | | Days after injection | | Number |
|---|---|---|---|---|
| Group | Dosage | before euthanization | | of mice |
| 1 | Vehicle (saline) | 3 | days | 3 |
| 2 | Vehicle (saline) | 7 | days | 3 |
| 3 | Vehicle (saline) | 14 | days | 3 |
| 4 | Vehicle (saline) | 28 | days | 3 |
| 5 | DTX-P-060 | 3 | days | 3 |
| 6 | DTX-P-060 | 7 | days | 3 |
| 7 | DTX-P-060 | 14 | days | 3 |
| 8 | DTX-P-060 | 28 | days | 3 |
| 9 | DTX-C-008 | 3 | days | 3 |
| 10 | DTX-C-008 | 7 | days | 3 |
| 11 | DTX-C-008 | 14 | days | 3 |
| 12 | DTX-C-008 | 28 | days | 3 |

Mice treated with the DTX-C-008 complex demonstrated approximately 50% reduction in DMPK expression in gastrocnemius (FIG. 11A) and tibialis anterior (FIG. 11B) muscles for all of Groups 9-12 (3-28 days between injection and euthanization), relative to vehicle. Mice treated with the DTX-P-060 naked oligonucleotide did not demonstrate significant reduction in DMPK expression.

These data indicate that the DTX-C-008 complex was capable of providing persistent reduction in DMPK expression for up to 28 days following dosage of mice with said DTX-C-008 complex.

### Example 7: Evaluation of Antisense Oligonucleotides that Target DMPK in Immortalized Myoblasts

Two hundred and thirty-six oligonucleotides for targeting DMPK were generated using in silico analysis. Each individual oligonucleotide was evaluated for their ability to target DMPK in cellulo at two doses −0.5 nM (low dose) and 50 nM (high dose).

Briefly, DM1 C15 immortalized myoblasts were cultured in T-75 flasks until near confluency (˜80% confluent). Myoblasts were then disrupted with trypsin and seeded into 96-well microplates at a density of 50,000 cells/well. Cells were allowed to recover overnight before the growth media was washed out and replaced with a no-serum media to induce differentiation into myotubes. Differentiation proceeded for seven days prior to treatment with DMPK-targeting oligonucleotides.

On day seven following induction of differentiation, DM1 C15 myotubes were transfected with an individual oligonucleotide using 0.3 μL of Lipofectamine MessengerMax per well. All oligonucleotides were tested at both 0.5 nM and 50 nM final concentrations in biological triplicates. After treatment with oligonucleotides, cells were incubated for 72 hours prior to being harvested for total RNA. cDNA was synthesized from the total RNA extracts and qPCR was performed to determine expression levels of DMPK in technical quadruplicate. All qPCR data were analyzed using a traditional AACT method and were normalized to a plate-based negative control that comprised cells treated with vehicle control (0.3 μL/well Lipofectamine MessengerMax without any oligonucleotide). Results from these experiments are shown in Table 4. ‘Normalized DMPK Remaining’ for each antisense oligonucleotide in Table 4 refers to the expression level of DMPK in cell treated with said antisense oligonucleotide relative to the negative control that comprised cells treated with vehicle control (wherein the expression level of the negative control has been normalized to equal 1.00)

The majority of tested DMPK-targeting antisense oligonucleotides demonstrated a reduction in DMPK expression in differentiated myotubes at both the low and high dose concentrations (0.5 nM and 50 nM, respectively). These data demonstrate that the antisense oligonucleotides shown in Table 4 are capable of targeting DMPK in cellulo, suggesting that muscle-targeting complexes comprising these antisense oligonucleotides would be capable of targeting DMPK in muscle tissues in vivo.

TABLE 4
Ability of DMPK-targeting antisense oligonucleotides to reduce expression of DMPK
in cellulo

| | | | | 0.5 nM | | 50 nM | |
|---|---|---|---|---|---|---|---|
| Antisense | SEQ | | SEQ | Normalized | Percent | Normalized | Percent |
| Oligonucleotide | ID | DMPK Target | ID | DMPK | DMPK | DMPK | DMPK |
| Sequence | NO: | Sequence | NO: | Remaining | Reduction | Remaining | Reduction |
| GGACGGCCCGGC | 45 | GGCAGCAAGCCG | 281 | 0.42 | 58.25 | 0.31 | 69.30 |
| UUGCUGCC | | GGCCGTCC | | | | | |
| GGGCCCGGAUCA | 46 | CAGTCCTGTGATC | 282 | 0.42 | 57.97 | 0.38 | 61.96 |
| CAGGACUG | | CGGGCCC | | | | | |
| CAAACUUGCUCA | 47 | GACACTGCTGAG | 283 | 0.69 | 31.45 | 0.46 | 53.93 |
| GCAGUGUC | | CAAGTTTG | | | | | |
| AAACUUGCUCAG | 48 | TGACACTGCTGA | 284 | 0.69 | 30.85 | 0.49 | 50.69 |
| CAGUGUCA | | GCAAGTTT | | | | | |
| CGGAUGGCCUCC | 49 | CGGGAGATGGAG | 285 | 0.71 | 28.92 | 0.44 | 55.57 |
| AUCUCCCG | | GCCATCCG | | | | | |
| CUCGGCCGGAAU | 50 | GGGAGCGGATTC | 286 | 0.71 | 28.64 | 0.35 | 64.75 |
| CCGCUCCC | | CGGCCGAG | | | | | |
| UCUCGGCCGGAA | 51 | GGAGCGGATTCC | 287 | 0.72 | 27.88 | 0.33 | 67.46 |
| UCCGCUCC | | GGCCGAGA | | | | | |
| UGCUCAGCAGUG | 52 | CCTGCTGACACTG | 288 | 0.73 | 27.08 | 0.34 | 65.78 |
| UCAGCAGG | | CTGAGCA | | | | | |
| UUGUCGGGUUUG | 53 | AGGGACATCAAA | 289 | 0.66 | 34.16 | 0.44 | 55.56 |
| AUGUCCCU | | CCCGACAA | | | | | |
| GUUGCGGGUUUG | 54 | GGGACATCAAAC | 290 | 0.67 | 33.31 | 0.39 | 61.07 |
| AUGUCCC | | CCGACAAC | | | | | |
| UCCGCCAGGUAG | 55 | GCGCGCTTCTACC | 291 | 0.72 | 27.99 | 0.20 | 80.06 |
| AAGCGCGC | | TGGCGGA | | | | | |
| CAUGGCAUACAC | 56 | CGGGCCAGGTGT | 292 | 0.68 | 31.63 | 0.26 | 74.03 |
| CUGGCCCG | | ATGCCATG | | | | | |
| AACUUGCUCAGC | 57 | CTGACACTGCTG | 293 | 0.80 | 19.81 | 0.47 | 52.64 |
| AGUGUCAG | | AGCAAGTT | | | | | |
| CAGCUGCGUGAU | 58 | GGCGGTGGATCA | 294 | 0.81 | 19.03 | 0.32 | 68.34 |
| CCACCGCC | | CGCAGCTG | | | | | |
| CGAAUGUCCGAC | 59 | GAGACACTGTCG | 295 | 0.60 | 40.21 | 0.36 | 64.42 |
| AGUGUCUC | | GACATTCG | | | | | |
| GAAGUCGGCCAG | 60 | ACATCCGCCTGG | 296 | 0.82 | 18.36 | 0.56 | 44.04 |
| GCGGAUGU | | CCGACTTC | | | | | |
| UGUCGGGUUUGA | 61 | CAGGGACATCAA | 297 | 0.70 | 30.09 | 0.32 | 68.14 |
| UGUCCCUG | | ACCCGACA | | | | | |
| GGAUGGCCUCCA | 62 | CCGGGAGATGGA | 298 | 0.75 | 24.93 | 0.39 | 60.77 |
| UCUCCCGG | | GGCCATCC | | | | | |
| AGGAUGUUGUCG | 63 | ATCAAACCCGAC | 299 | 0.76 | 24.19 | 0.61 | 39.48 |
| GGUUUGAU | | AACATCCT | | | | | |
| GUCGGGUUUGAU | 64 | ACAGGGACATCA | 300 | 0.71 | 28.89 | 0.36 | 64.15 |
| GUCCCUGU | | AACCCGAC | | | | | |
| AAUACUCCAUGA | 65 | GTACCTGGTCATG | 301 | 0.71 | 28.86 | 0.48 | 52.07 |
| CCAGGUAC | | GAGTATT | | | | | |
| CUUGUUCAUGAU | 66 | CCATGAAGATCA | 302 | 0.84 | 16.06 | 0.51 | 49.47 |
| CUUCAUGG | | TGAACAAG | | | | | |
| UCAGUGCAUCCA | 67 | CCACGTTTTGGAT | 303 | 0.84 | 15.76 | 0.58 | 42.06 |
| AAACGUGG | | GCACTGA | | | | | |
| CUGUCCCGGAGA | 68 | TGGGATGGTCTCC | 304 | 0.64 | 35.85 | 0.49 | 50.78 |
| CCAUCCCA | | GGGACAG | | | | | |
| GGGCCUGGGACC | 69 | GACAGTGAGGTC | 305 | 0.63 | 37.19 | 0.23 | 76.81 |
| UCACUGUC | | CCAGGCCC | | | | | |
| CCCACGUAAUAC | 70 | GTCATGGAGTATT | 306 | 0.72 | 28.21 | 0.54 | 45.94 |
| UCCAUGAC | | ACGTGGG | | | | | |
| CUCUGCCGCAGG | 71 | CGGCTGTCCCTGC | 307 | 0.63 | 37.09 | 0.06 | 93.59 |
| GACAGCCG | | GGCAGAG | | | | | |
| CUGUGCACGUAG | 72 | CGGCTTGGCTAC | 308 | 0.74 | 25.67 | 0.30 | 70.10 |
| CCAAGCCG | | GTGCACAG | | | | | |
| UGCCCAUCCACG | 73 | GGCCCTGACGTG | 309 | 0.86 | 13.63 | 0.67 | 33.09 |
| UCAGGGCC | | GATGGGCA | | | | | |
| AGCGCCUCCGAU | 74 | CCTGGCCTATCGG | 310 | 0.79 | 21.19 | 0.38 | 61.91 |
| AGGCCAGG | | AGGCGCT | | | | | |
| UGUGCACGUAGC | 75 | CCGGCTTGGCTAC | 311 | 0.75 | 24.74 | 0.25 | 75.09 |
| CAAGCCGG | | GTGCACA | | | | | |
| GACCAGGUACAG | 76 | AGAACTACCTGT | 312 | 0.57 | 42.85 | 0.29 | 70.95 |
| GUAGUUCU | | ACCTGGTC | | | | | |
| CCAUCUCGGCCG | 77 | GCGGATTCCGGC | 313 | 0.79 | 20.50 | 0.40 | 59.76 |
| GAAUCCGC | | CGAGATGG | | | | | |
| CAUCUCGGCCGG | 78 | AGCGGATTCCGG | 314 | 0.80 | 20.21 | 0.41 | 59.40 |
| AAUCCGCU | | CCGAGATG | | | | | |
| UUGCCAUAGGUC | 79 | ACGGCGGAGACC | 315 | 0.64 | 36.30 | 0.40 | 60.12 |
| UCCGCCGU | | TATGGCAA | | | | | |
| ACAGCGGUCCAG | 80 | ACATCCTGCTGG | 316 | 0.80 | 19.94 | 0.45 | 55.14 |
| CAGGAUGU | | ACCGCTGT | | | | | |
| AAAGCGCCUCCG | 81 | TGGCCTATCGGA | 317 | 0.80 | 19.89 | 0.38 | 62.04 |
| AUAGGCCA | | GGCGCTTT | | | | | |
| GCCAAAGAAGAA | 82 | CACATCCCTTCTT | 318 | 0.75 | 24.87 | 0.44 | 56.19 |
| GGGAUGUG | | CTTTGGC | | | | | |
| CACGUAAUACUC | 83 | TGGTCATGGAGT | 319 | 0.76 | 24.40 | 0.54 | 46.50 |
| CAUGACCA | | ATTACGTG | | | | | |
| AUCUCGGCCGGA | 84 | GAGCGGATTCCG | 320 | 0.88 | 11.61 | 0.34 | 65.98 |
| AUCCGCUC | | GCCGAGAT | | | | | |
| GCUUCAUCUUCA | 85 | AGCGGTAGTGAA | 321 | 0.69 | 31.44 | 0.48 | 51.78 |
| CUACCGCU | | GATGAAGC | | | | | |
| GCCAUCUCGGCC | 86 | CGGATTCCGGCC | 322 | 0.81 | 18.56 | 0.14 | 86.39 |
| GGAAUCCG | | GAGATGGC | | | | | |
| CAGGGACAGCCG | 87 | AGTTCCAGCGGC | 323 | 0.68 | 32.09 | 0.41 | 58.84 |
| CUGGAACU | | TGTCCCTG | | | | | |
| AUGACAAUCUCC | 88 | TACCTGGCGGAG | 324 | 0.58 | 42.38 | 0.40 | 60.47 |
| GCCAGGUA | | ATTGTCAT | | | | | |
| GGCCAUGACAAU | 89 | TGGCGGAGATTG | 325 | 0.58 | 42.38 | 0.25 | 75.00 |
| CUCCGCCA | | TCATGGCC | | | | | |
| AUACUCCAUGAC | 90 | TGTACCTGGTCAT | 326 | 0.77 | 23.07 | 0.43 | 56.84 |
| CAGGUACA | | GGAGTAT | | | | | |
| GCCUCUGCCUCG | 91 | CAACTACGCGAG | 327 | 0.65 | 35.38 | 0.19 | 81.18 |
| CGUAGUUG | | GCAGAGGC | | | | | |
| GAAUGUCCGACA | 92 | GGAGACACTGTC | 328 | 0.70 | 30.09 | 0.37 | 63.41 |
| GUGUCUCC | | GGACATTC | | | | | |
| CGUUCCAUCUGC | 93 | AGCTGCGGGCAG | 329 | 0.66 | 33.74 | 0.31 | 68.72 |
| CCGCAGCU | | ATGGAACG | | | | | |
| CCUUGUAGUGGA | 94 | CAAGATCGTCCA | 330 | 0.83 | 17.20 | 0.34 | 65.91 |
| CGAUCUUG | | CTACAAGG | | | | | |
| AUCUCCGCCAGG | 95 | CGCTTCTACCTGG | 331 | 0.58 | 42.37 | 0.35 | 65.50 |
| UAGAAGCG | | CGGAGAT | | | | | |
| CUCAGGCUCUGC | 96 | CTCACCCGGCAG | 332 | 0.70 | 30.13 | 0.37 | 63.07 |
| CGGGUGAG | | AGCCTGAG | | | | | |
| UGCUUCAUCUUC | 97 | GCGGTAGTGAAG | 333 | 0.71 | 28.82 | 0.40 | 60.24 |
| ACUACCGC | | ATGAAGCA | | | | | |
| GCAGGAUGUUGU | 98 | CAAACCCGACAA | 334 | 0.56 | 44.39 | 0.22 | 78.03 |
| CGGGUUUG | | CATCCTGC | | | | | |
| GGCCUCAGCCUC | 99 | CTGCGGCAGAGG | 335 | 0.80 | 20.12 | 0.29 | 71.28 |
| UGCCGCAG | | CTGAGGCC | | | | | |
| UGUUGUCGGGUU | 100 | GGACATCAAACC | 336 | 0.79 | 21.00 | 0.58 | 42.19 |
| UGAUGUCC | | CGACAACA | | | | | |
| CCACGUAAUACU | 101 | GGTCATGGAGTA | 337 | 0.79 | 20.84 | 0.50 | 50.06 |
| CCAUGACC | | TTACGTGG | | | | | |
| CCGUUCCAUCUG | 102 | GCTGCGGGCAGA | 338 | 0.68 | 31.74 | 0.23 | 77.46 |
| CCCGCAGC | | TGGAACGG | | | | | |
| UUCCCGAGUAAG | 103 | TCTGCCTGCTTAC | 339 | 0.69 | 31.49 | 0.50 | 49.81 |
| CAGGCAGA | | TCGGGAA | | | | | |
| UGAUCUUCAUGG | 104 | GGTGTATGCCAT | 340 | 0.72 | 27.70 | 0.10 | 89.68 |
| CAUACACC | | GAAGATCA | | | | | |
| AGGGACAGCCGC | 105 | CAGTTCCAGCGG | 341 | 0.71 | 28.72 | 0.55 | 45.34 |
| UGGAACTG | | CTGTCCCT | | | | | |
| GGGUUUGAUGUC | 106 | TGCACAGGGACA | 342 | 0.60 | 40.12 | 0.37 | 62.61 |
| CCUGUGCA | | TCAAACCC | | | | | |
| UGACAAUCUCCG | 107 | CTACCTGGCGGA | 343 | 0.61 | 38.86 | 0.33 | 66.56 |
| CCAGGUAG | | GATTGTCA | | | | | |
| CACAGCGGUCCA | 108 | CATCCTGCTGGAC | 344 | 0.93 | 6.62 | 0.40 | 59.58 |
| GCAGGAUG | | CGCTGTG | | | | | |
| GCGUAGAAGGGC | 109 | GGGCAGACGCCC | 345 | 0.60 | 39.53 | 0.22 | 77.91 |
| GUCUGCCC | | TTCTACGC | | | | | |
| CUCAGCCUCUGC | 110 | TCCCTGCGGCAG | 346 | 0.82 | 17.86 | 0.20 | 79.58 |
| CGCAGGGA | | AGGCTGAG | | | | | |
| GUCUCAGUGCAU | 111 | CGTTTTGGATGCA | 347 | 0.81 | 18.85 | 0.54 | 46.13 |
| CCAAAACG | | CTGAGAC | | | | | |
| GGACGAUCUUGC | 112 | GACCTATGGCAA | 348 | 0.70 | 29.82 | 0.51 | 48.97 |
| CAUAGGUC | | GATCGTCC | | | | | |
| UCAGCAGUGUCA | 113 | GGACCTGCTGAC | 349 | 0.67 | 33.46 | 0.39 | 61.11 |
| GCAGGUCC | | ACTGCTGA | | | | | |
| GCUCCUGGGCGG | 114 | GTCTGGCGCCGC | 350 | 0.91 | 8.52 | 0.21 | 78.79 |
| CGCCAGAC | | CCAGGAGC | | | | | |
| AGCAGGAUGUUG | 115 | AAACCCGACAAC | 351 | 0.59 | 41.05 | 0.26 | 74.02 |
| UCGGGUUU | | ATCCTGCT | | | | | |
| AUCCGCUCCUGC | 116 | CGGCAGTTGCAG | 352 | 0.87 | 12.80 | 0.60 | 40.06 |
| AACUGCCG | | GAGCGGAT | | | | | |
| AGGAGCAGGGAA | 117 | GAGGCGCTTTCCC | 353 | 0.67 | 33.24 | 0.38 | 62.37 |
| AGCGCCUC | | TGCTCCT | | | | | |
| ACACCUGGCCCG | 118 | GAAGCAGACGGG | 354 | 0.67 | 33.00 | 0.45 | 55.40 |
| UCUGCUUC | | CCAGGTGT | | | | | |
| CCCAGCGCCCAC | 119 | TGTGACTGGTGG | 355 | 0.62 | 37.93 | 0.32 | 67.82 |
| CAGUCACA | | GCGCTGGG | | | | | |
| GCUCCCUCUGCC | 120 | TTGCTGCAGGCA | 356 | 0.74 | 26.41 | 0.30 | 70.15 |
| UGCAGCAA | | GAGGGAGC | | | | | |
| GCUCAGGCUCUG | 121 | TCACCCGGCAGA | 357 | 0.74 | 25.69 | 0.39 | 60.71 |
| CCGGGUGA | | GCCTGAGC | | | | | |
| UUGAUGUCCCUG | 122 | TACGTGCACAGG | 358 | 0.74 | 25.67 | 0.45 | 55.13 |
| UGCACGUA | | GACATCAA | | | | | |
| GCCUCAGCCUCU | 123 | CCTGCGGCAGAG | 359 | 0.84 | 16.37 | 0.54 | 46.42 |
| GCCGCAGG | | GCTGAGGC | | | | | |
| GGUAGUUCUCAU | 124 | CTTCCAGGATGA | 360 | 0.75 | 25.48 | 0.44 | 56.15 |
| CCUGGAAG | | GAACTACC | | | | | |
| CAGCGCCCACCA | 125 | AGTGTGACTGGT | 361 | 0.63 | 37.28 | 0.35 | 64.93 |
| GUCACACU | | GGGCGCTG | | | | | |
| CCCAAACUUGCU | 126 | CACTGCTGAGCA | 362 | 0.63 | 37.02 | 0.38 | 61.78 |
| CAGCAGUG | | AGTTTGGG | | | | | |
| CUUGCCAUAGGU | 127 | CGGCGGAGACCT | 363 | 0.73 | 27.04 | 0.29 | 71.05 |
| CUCCGCCG | | ATGGCAAG | | | | | |
| UACACCUGGCCC | 128 | AAGCAGACGGGC | 364 | 0.69 | 31.10 | 0.43 | 57.43 |
| GUCUGCUU | | CAGGTGTA | | | | | |
| CCAGCGCCCACC | 129 | GTGTGACTGGTG | 365 | 0.64 | 36.17 | 0.29 | 70.96 |
| AGUCACAC | | GGCGCTGG | | | | | |
| GGCCUCAGCCUG | 130 | CTTTCGGCCAGGC | 366 | 0.86 | 14.49 | 0.35 | 64.80 |
| GCCGAAAG | | TGAGGCC | | | | | |
| AAUCUCCGCCAG | 131 | GCTTCTACCTGGC | 367 | 0.64 | 35.85 | 0.35 | 65.27 |
| GUAGAAGC | | GGAGATT | | | | | |
| AUGGCAUACACC | 132 | ACGGGCCAGGTG | 368 | 0.86 | 14.31 | 0.50 | 49.63 |
| UGGCCCGU | | TATGCCAT | | | | | |
| CCAUGACAAUCU | 133 | CCTGGCGGAGAT | 369 | 0.65 | 34.53 | 0.24 | 76.46 |
| CCGCCAGG | | TGTCATGG | | | | | |
| UCCCCAAACUUG | 134 | CTGCTGAGCAAG | 370 | 0.94 | 5.73 | 0.55 | 44.67 |
| CUCAGCAG | | TTTGGGGA | | | | | |
| GAUGUUGUCGGG | 135 | ACATCAAACCCG | 371 | 0.90 | 10.06 | 0.58 | 42.42 |
| UUUGAUGU | | ACAACATC | | | | | |
| GUUUGCCCAUCC | 136 | CCTGACGTGGAT | 372 | 0.66 | 34.36 | 0.46 | 54.49 |
| ACGUCAGG | | GGGCAAAC | | | | | |
| CGGACGGCCCGG | 137 | GCAGCAAGCCGG | 373 | 0.95 | 5.42 | 0.70 | 30.41 |
| CUUGCUGC | | GCCGTCCG | | | | | |
| CUCCGCCAGGUA | 138 | CGCGCTTCTACCT | 374 | 0.70 | 30.22 | 0.22 | 78.14 |
| GAAGCGCG | | GGCGGAG | | | | | |
| GUACAGGUAGUU | 139 | AGGATGAGAACT | 375 | 0.68 | 31.52 | 0.34 | 65.57 |
| CUCAUCCU | | ACCTGTAC | | | | | |
| AGGGCGUCUGCC | 140 | GTTCTATGGGCA | 376 | 0.87 | 13.23 | 0.41 | 58.98 |
| CAUAGAAC | | GACGCCCT | | | | | |
| UGGCCACAGCGG | 141 | CTGCTGGACCGCT | 377 | 0.70 | 29.59 | 0.31 | 69.44 |
| UCCAGCAG | | GTGGCCA | | | | | |
| CGUAGUUGACUG | 142 | AACTTCGCCAGTC | 378 | 0.75 | 25.26 | 0.38 | 61.52 |
| GCGAAGUU | | AACTACG | | | | | |
| UCUGCCGCAGGG | 143 | GCGGCTGTCCCTG | 379 | 0.77 | 22.97 | 0.18 | 82.10 |
| ACAGCCGC | | CGGCAGA | | | | | |
| AAGCGCCUCCGA | 144 | CTGGCCTATCGG | 380 | 0.91 | 8.91 | 0.56 | 43.93 |
| UAGGCCAG | | AGGCGCTT | | | | | |
| GACAGAACAACG | 145 | CTGTTCGCCGTTG | 381 | 0.79 | 21.41 | 0.30 | 70.49 |
| GCGAACAG | | TTCTGTC | | | | | |
| GCUCAGCAGUGU | 146 | ACCTGCTGACACT | 382 | 0.71 | 29.18 | 0.27 | 73.46 |
| CAGCAGGU | | GCTGAGC | | | | | |
| AUGAUCUUCAUG | 147 | GTGTATGCCATG | 383 | 0.87 | 12.76 | 0.60 | 39.97 |
| GCAUACAC | | AAGATCAT | | | | | |
| UUUGCCCAUCCA | 148 | CCCTGACGTGGA | 384 | 0.67 | 32.79 | 0.41 | 59.36 |
| CGUCAGGG | | TGGGCAAA | | | | | |
| ACUUGCUCAGCA | 149 | GCTGACACTGCT | 385 | 0.72 | 27.84 | 0.39 | 60.71 |
| GUGUCAGC | | GAGCAAGT | | | | | |
| UGAUGUCCCUGU | 150 | CTACGTGCACAG | 386 | 0.79 | 20.58 | 0.41 | 59.00 |
| GCACGUAG | | GGACATCA | | | | | |
| AAAUACCGAGGA | 151 | CCCGACATTCCTC | 387 | 0.89 | 11.25 | 0.49 | 50.91 |
| AUGUCGGG | | GGTATTT | | | | | |
| GGCGAAUACACC | 152 | GGGCGCTGGGTG | 388 | 0.80 | 19.77 | 0.31 | 68.72 |
| CAGCGCCC | | TATTCGCC | | | | | |
| AGACAAUAAAUA | 153 | TTCCTCGGTATTT | 389 | 0.71 | 29.37 | 0.52 | 48.20 |
| CCGAGGAA | | ATTGTCT | | | | | |
| CCCGUCUGCUUC | 154 | GTGAAGATGAAG | 390 | 0.80 | 20.31 | 0.56 | 43.97 |
| AUCUUCAC | | CAGACGGG | | | | | |
| CUGCCUGCAGCA | 155 | GATGGAGTTGCT | 391 | 0.77 | 23.10 | 0.53 | 46.69 |
| ACUCCAUC | | GCAGGCAG | | | | | |
| CCUCAGCCUCUG | 156 | CCCTGCGGCAGA | 392 | 0.89 | 10.87 | 0.45 | 55.22 |
| CCGCAGGG | | GGCTGAGG | | | | | |
| GUGUCCGGAAGU | 157 | AGCAGGCGACTT | 393 | 0.77 | 22.99 | 0.26 | 73.65 |
| CGCCUGCU | | CCGGACAC | | | | | |
| UGCACGUGUGGC | 158 | CTGCTTGAGCCAC | 394 | 0.89 | 10.81 | 0.36 | 64.18 |
| UCAAGCAG | | ACGTGCA | | | | | |
| GACAAUAAAUAC | 159 | ATTCCTCGGTATT | 395 | 0.71 | 28.97 | 0.52 | 47.51 |
| CGAGGAAU | | TATTGTC | | | | | |
| GCCAUGACAAUC | 160 | CTGGCGGAGATT | 396 | 0.69 | 30.96 | 0.19 | 81.00 |
| UCCGCCAG | | GTCATGGC | | | | | |
| GCUGUCCCGGAG | 161 | GGGATGGTCTCC | 397 | 0.77 | 22.57 | 0.34 | 66.27 |
| ACCAUCCC | | GGGACAGC | | | | | |
| CAUGACCAGGUA | 162 | ACTACCTGTACCT | 398 | 0.81 | 19.39 | 0.41 | 59.09 |
| CAGGUAGU | | GGTCATG | | | | | |
| AGCGCCCACCAG | 163 | GAGTGTGACTGG | 399 | 0.70 | 30.36 | 0.36 | 63.67 |
| UCACACUC | | TGGGCGCT | | | | | |
| UCUCAGUGCAUC | 164 | ACGTTTTGGATGC | 400 | 0.89 | 10.88 | 0.49 | 51.34 |
| CAAAACGU | | ACTGAGA | | | | | |
| UUUGGGCAGAUG | 165 | AGGCCCTCCATCT | 401 | 0.65 | 35.14 | 0.30 | 70.00 |
| GAGGGCCU | | GCCCAAA | | | | | |
| GAUGUCCCUGUG | 166 | GCTACGTGCACA | 402 | 0.81 | 18.99 | 0.38 | 62.46 |
| CACGUAGC | | GGGACATC | | | | | |
| CAGCAGUGUCAG | 167 | GGGACCTGCTGA | 403 | 0.74 | 25.67 | 0.48 | 51.97 |
| CAGGUCCC | | CACTGCTG | | | | | |
| CAUGACAAUCUC | 168 | ACCTGGCGGAGA | 404 | 0.71 | 29.45 | 0.29 | 70.52 |
| CGCCAGGU | | TTGTCATG | | | | | |
| ACUUGUUCAUGA | 169 | CATGAAGATCAT | 405 | 0.75 | 25.47 | 0.47 | 52.89 |
| UCUUCAUG | | GAACAAGT | | | | | |
| GUGGAAUCCGCG | 170 | CCCTTCTACGCGG | 406 | 0.69 | 30.55 | 0.51 | 49.34 |
| UAGAAGGG | | ATTCCAC | | | | | |
| UGGCCAUGACAA | 171 | GGCGGAGATTGT | 407 | 0.70 | 30.46 | 0.27 | 72.55 |
| UCUCCGCC | | CATGGCCA | | | | | |
| GGGACAGACAAU | 172 | CGGTATTTATTGT | 408 | 0.73 | 27.19 | 0.49 | 50.50 |
| AAAUACCG | | CTGTCCC | | | | | |
| CCGCUCCCCAAA | 173 | TGAGCAAGTTTG | 409 | 1.00 | 0.28 | 0.43 | 56.82 |
| CUUGCUCA | | GGGAGCGG | | | | | |
| CGGCUCAGGCUC | 174 | ACCCGGCAGAGC | 410 | 0.82 | 17.97 | 0.31 | 69.03 |
| UGCCGGGU | | CTGAGCCG | | | | | |
| GGCUCCUGGGCG | 175 | TCTGGCGCCGCCC | 411 | 1.00 | 0.05 | 0.04 | 96.23 |
| GCGCCAGA | | AGGAGCC | | | | | |
| UUUCCCGAGUAA | 176 | CTGCCTGCTTACT | 412 | 0.79 | 20.69 | 0.55 | 44.89 |
| GCAGGCAG | | CGGGAAA | | | | | |
| GGAUGUUGUCGG | 177 | CATCAAACCCGA | 413 | 0.96 | 4.26 | 0.59 | 40.81 |
| GUUUGAUG | | CAACATCC | | | | | |
| CAGGUAGUUCUC | 178 | TCCAGGATGAGA | 414 | 0.74 | 25.92 | 0.23 | 76.71 |
| AUCCUGGA | | ACTACCTG | | | | | |
| UGCCCAUAGAAC | 179 | TATGAAATGTTCT | 415 | 0.92 | 7.67 | 0.65 | 34.56 |
| AUUUCAUA | | ATGGGCA | | | | | |
| UAGUUCUCAUCC | 180 | GCCTTCCAGGAT | 416 | 0.83 | 16.83 | 0.56 | 43.88 |
| UGGAAGGC | | GAGAACTA | | | | | |
| AUGUCCCUGUGC | 181 | GGCTACGTGCAC | 417 | 0.83 | 16.78 | 0.51 | 49.29 |
| ACGUAGCC | | AGGGACAT | | | | | |
| CGGGCCCGGAUC | 182 | AGTCCTGTGATCC | 418 | 0.83 | 17.45 | 0.33 | 67.11 |
| ACAGGACU | | GGGCCCG | | | | | |
| UGGACGAUCUUG | 183 | ACCTATGGCAAG | 419 | 0.81 | 19.20 | 0.57 | 42.52 |
| CCAUAGGU | | ATCGTCCA | | | | | |
| GUUGGCCGGCGU | 184 | GGTGGCCCACGC | 420 | 1.02 | −1.82 | 0.56 | 43.57 |
| GGGCCACC | | CGGCCAAC | | | | | |
| CUCAGUGCAUCC | 185 | CACGTTTTGGATG | 421 | 0.92 | 7.65 | 0.46 | 54.26 |
| AAAACGUG | | CACTGAG | | | | | |
| UCGAAGUUGCAU | 186 | ACCGACACATGC | 422 | 0.77 | 22.96 | 0.42 | 58.15 |
| GUGUCGGU | | AACTTCGA | | | | | |
| UGGAACACGGAC | 187 | GCCGGGCCGTCC | 423 | 1.02 | −1.90 | 0.39 | 60.96 |
| GGCCCGGC | | GTGTTCCA | | | | | |
| CCGAGAGCAGCG | 188 | CTCACTTGCGCTG | 424 | 0.84 | 16.13 | 0.59 | 40.93 |
| CAAGUGAG | | CTCTCGG | | | | | |
| UCCUGCAACUGC | 189 | CACGTCCGGCAG | 425 | 0.84 | 16.06 | 0.55 | 44.61 |
| CGGACGUG | | TTGCAGGA | | | | | |
| UCACCAACACGU | 190 | GGAGAGGGACGT | 426 | 0.53 | 47.12 | 0.16 | 84.09 |
| CCCUCUCC | | GTTGGTGA | | | | | |
| UGCCUGCAGCAA | 191 | GGATGGAGTTGC | 427 | 0.86 | 13.99 | 0.50 | 49.75 |
| CUCCAUCC | | TGCAGGCA | | | | | |
| UUGGCCGGCGUG | 192 | TGGTGGCCCACG | 428 | 1.03 | −3.19 | 0.56 | 44.37 |
| GGCCACCA | | CCGGCCAA | | | | | |
| GAGCCUCUGCCU | 193 | ACTACGCGAGGC | 429 | 0.81 | 18.77 | 0.22 | 77.78 |
| CGCGUAGU | | AGAGGCTC | | | | | |
| AAGGGCGUCUGC | 194 | TTCTATGGGCAG | 430 | 0.87 | 13.15 | 0.65 | 34.56 |
| CCAUAGAA | | ACGCCCTT | | | | | |
| ACAGACAAUAAA | 195 | CCTCGGTATTTAT | 431 | 1.04 | −3.95 | 0.26 | 74.02 |
| UACCGAGG | | TGTCTGT | | | | | |
| GGACAGACAAUA | 196 | TCGGTATTTATTG | 432 | 0.77 | 22.57 | 0.47 | 52.51 |
| AAUACCGA | | TCTGTCC | | | | | |
| ACGUGUGCCUCU | 197 | CGGGACCTAGAG | 433 | 0.84 | 16.47 | 0.22 | 77.73 |
| AGGUCCCG | | GCACACGT | | | | | |
| GGCACGAGACAG | 198 | CCGTTGTTCTGTC | 434 | 0.84 | 16.10 | 0.32 | 68.01 |
| AACAACGG | | TCGTGCC | | | | | |
| UGACCAGGUACA | 199 | GAACTACCTGTA | 435 | 0.78 | 22.00 | 0.36 | 63.73 |
| GGUAGUUC | | CCTGGTCA | | | | | |
| CUCUGCCGGGUG | 200 | GAGGTGCTCACC | 436 | 0.75 | 25.25 | 0.26 | 74.36 |
| AGCACCUC | | CGGCAGAG | | | | | |
| GACAAUCUCCGC | 201 | TCTACCTGGCGG | 437 | 0.76 | 23.70 | 0.50 | 49.82 |
| CAGGUAGA | | AGATTGTC | | | | | |
| UCUCCGCCAGGU | 202 | GCGCTTCTACCTG | 438 | 0.80 | 19.59 | 0.33 | 66.52 |
| AGAAGCGC | | GCGGAGA | | | | | |
| CUCUGCCUCGCG | 203 | GTCAACTACGCG | 439 | 0.83 | 16.61 | 0.09 | 91.21 |
| UAGUUGAC | | AGGCAGAG | | | | | |
| CUUUGGGCAGAU | 204 | GGCCCTCCATCTG | 440 | 0.72 | 28.06 | 0.33 | 67.50 |
| GGAGGGCC | | CCCAAAG | | | | | |
| ACAGGUAGUUCU | 205 | CCAGGATGAGAA | 441 | 0.79 | 20.51 | 0.15 | 85.36 |
| CAUCCUGG | | CTACCTGT | | | | | |
| CCAAACUUGCTC | 206 | ACACTGCTGAGC | 442 | 0.76 | 23.64 | 0.42 | 57.70 |
| AGCAGUGU | | AAGTTTGG | | | | | |
| UCGGGUUUGAUG | 207 | CACAGGGACATC | 443 | 0.78 | 22.49 | 0.43 | 57.16 |
| UCCCUGUG | | AAACCCGA | | | | | |
| GGCUUGCUGCCU | 208 | GCCTGGGAAGGC | 444 | 1.06 | −6.32 | 0.52 | 48.15 |
| UCCCAGGC | | AGCAAGCC | | | | | |
| UACAGGUAGUUC | 209 | CAGGATGAGAAC | 445 | 0.80 | 19.83 | 0.27 | 72.51 |
| UCAUCCUG | | TACCTGTA | | | | | |
| UUGCCCAUCCAC | 210 | GCCCTGACGTGG | 446 | 0.78 | 22.23 | 0.33 | 67.15 |
| GUCAGGGC | | ATGGGCAA | | | | | |
| AGGUACAGGUAG | 211 | GATGAGAACTAC | 447 | 0.81 | 18.68 | 0.41 | 58.92 |
| UUCUCAUC | | CTGTACCT | | | | | |
| GACAGACAAUAA | 212 | CTCGGTATTTATT | 448 | 0.82 | 18.26 | 0.62 | 38.07 |
| AUACCGAG | | GTCTGTC | | | | | |
| UAGAACAUUUCA | 213 | TTCGCCTATGAAA | 449 | 0.80 | 20.23 | 0.56 | 43.67 |
| UAGGCGAA | | TGTTCTA | | | | | |
| AGGGCCUUUUAU | 214 | CCTCGCGAATAA | 450 | 0.86 | 13.63 | 0.34 | 66.43 |
| UCGCGAGG | | AAGGCCCT | | | | | |
| GCCUCGCGUAGU | 215 | GCCAGTCAACTA | 451 | 0.87 | 12.98 | 0.09 | 91.10 |
| UGACUGGC | | CGCGAGGC | | | | | |
| CCAGCAGGAUGU | 216 | ACCCGACAACAT | 452 | 0.60 | 40.29 | 0.10 | 89.59 |
| UGUCGGGU | | CCTGCTGG | | | | | |
| GUAGUUGACUGG | 217 | GAACTTCGCCAG | 453 | 0.93 | 7.50 | 0.55 | 45.33 |
| CGAAGUUC | | TCAACTAC | | | | | |
| UGCGGAUGGCCU | 218 | GGAGATGGAGGC | 454 | 0.60 | 40.15 | 0.16 | 84.43 |
| CCAUCUCC | | CATCCGCA | | | | | |
| ACAAUCUCCGCC | 219 | TTCTACCTGGCGG | 455 | 0.81 | 19.09 | 0.50 | 49.75 |
| AGGUAGAA | | AGATTGT | | | | | |
| GCGAAUACACCC | 220 | TGGGCGCTGGGT | 456 | 0.93 | 6.94 | 0.30 | 69.72 |
| AGCGCCCA | | GTATTCGC | | | | | |
| GUAGUUCUCAUC | 221 | CCTTCCAGGATG | 457 | 0.93 | 7.43 | 0.45 | 55.09 |
| CUGGAAGG | | AGAACTAC | | | | | |
| GGCUCAGGCUCU | 222 | CACCCGGCAGAG | 458 | 0.93 | 7.38 | 0.34 | 65.82 |
| GCCGGGUG | | CCTGAGCC | | | | | |
| CCAUUCACCAAC | 223 | AGGGACGTGTTG | 459 | 0.61 | 39.26 | 0.13 | 86.83 |
| ACGUCCCU | | GTGAATGG | | | | | |
| ACCAGGUACAGG | 224 | GAGAACTACCTG | 460 | 0.84 | 16.09 | 0.23 | 76.96 |
| UAGUUCUC | | TACCTGGT | | | | | |
| CTGCAGUUUGCC | 225 | CGTGGATGGGCA | 461 | 1.11 | −10.69 | 0.40 | 60.08 |
| CAUCCACG | | AACTGCAG | | | | | |
| UUGUUCAUGAUC | 226 | GCCATGAAGATC | 462 | 0.86 | 14.13 | 0.55 | 45.23 |
| UUCAUGGC | | ATGAACAA | | | | | |
| UUGAUGUCCCUG | 227 | ACGTGCACAGGG | 463 | 0.93 | 6.92 | 0.57 | 43.07 |
| UGCACGU | | ACATCAAA | | | | | |
| GCGGUCCAGCAG | 228 | ACAACATCCTGCT | 464 | 0.61 | 38.84 | 0.16 | 83.64 |
| GAUGUUGU | | GGACCGC | | | | | |
| GUCUAUGGCCAU | 229 | AGATTGTCATGG | 465 | 1.11 | −11.00 | 0.27 | 73.11 |
| GACAAUCU | | CCATAGAC | | | | | |
| GGAGCAGGGAAA | 230 | GGAGGCGCTTTC | 466 | 0.79 | 21.46 | 0.12 | 88.35 |
| GCGCCUCC | | CCTGCTCC | | | | | |
| UGCCUCGCGUAG | 231 | CCAGTCAACTAC | 467 | 0.89 | 11.03 | 0.12 | 88.02 |
| UUGACUGG | | GCGAGGCA | | | | | |
| GCGGAUGGCCUC | 232 | GGGAGATGGAGG | 468 | 0.79 | 21.25 | 0.28 | 71.77 |
| CAUCUCCC | | CCATCCGC | | | | | |
| UUUCAUAGGCGA | 233 | GGGTGTATTCGCC | 469 | 0.94 | 5.56 | 0.47 | 53.28 |
| AUACACCC | | TATGAAA | | | | | |
| GCCUGUCAGCGA | 234 | CCTCCGACTCGCT | 470 | 0.89 | 10.81 | 0.24 | 75.67 |
| GUCGGAGG | | GACAGGC | | | | | |
| CCACUUCAGCUG | 235 | GGATGAAACAGC | 471 | 0.78 | 22.40 | 0.36 | 64.20 |
| UUUCAUCC | | TGAAGTGG | | | | | |
| CAUCCGCUCCUG | 236 | GGCAGTTGCAGG | 472 | 0.79 | 21.04 | 0.23 | 76.81 |
| CAACUGCC | | AGCGGATG | | | | | |
| UCUAGGGUUCAG | 237 | CGCGCTCCCTGA | 473 | 0.78 | 21.81 | 0.17 | 83.22 |
| GGAGCGCG | | ACCCTAGA | | | | | |
| CACCAACACGUC | 238 | AGGAGAGGGACG | 474 | 0.62 | 37.51 | 0.18 | 81.57 |
| CCUCUCCU | | TGTTGGTG | | | | | |
| CAGGAGCAGGGA | 239 | AGGCGCTTTCCCT | 475 | 0.88 | 12.48 | 0.48 | 51.82 |
| AAGCGCCU | | GCTCCTG | | | | | |
| CAAUCUCCGCCA | 240 | CTTCTACCTGGCG | 476 | 0.84 | 15.95 | 0.51 | 49.25 |
| GGUAGAAG | | GAGATTG | | | | | |
| AUGUUGUCGGGU | 241 | GACATCAAACCC | 477 | 0.83 | 16.93 | 0.47 | 52.83 |
| UUGAUGUC | | GACAACAT | | | | | |
| CCAUCCGCUCCU | 242 | GCAGTTGCAGGA | 478 | 0.80 | 19.53 | 0.28 | 71.62 |
| GCAACUGC | | GCGGATGG | | | | | |
| GCGUCACCUCGG | 243 | GGCTGAGGCCGA | 479 | 0.80 | 20.02 | 0.19 | 81.27 |
| CCUCAGCC | | GGTGACGC | | | | | |
| GAGGGCCUUUUA | 244 | CTCGCGAATAAA | 480 | 0.92 | 8.23 | 0.38 | 62.21 |
| UUCGCGAG | | AGGCCCTC | | | | | |
| AGCGGCAGAGAG | 245 | GAGCACCTCTCTC | 481 | 0.80 | 19.75 | 0.09 | 90.71 |
| AGGUGCUC | | TGCCGCT | | | | | |
| CAUCCAAAACGU | 246 | CCCAATCCACGTT | 482 | 0.81 | 19.12 | 0.22 | 77.98 |
| GGAUUGGG | | TTGGATG | | | | | |
| UUGGGCAGAUGG | 247 | AAGGCCCTCCAT | 483 | 0.81 | 19.08 | 0.22 | 78.39 |
| AGGGCCUU | | CTGCCCAA | | | | | |
| CCUCUGCCUCGC | 248 | TCAACTACGCGA | 484 | 0.93 | 7.39 | 0.15 | 85.33 |
| GUAGUUGA | | GGCAGAGG | | | | | |
| ACAGAACAACGG | 249 | CCTGTTCGCCGTT | 485 | 0.98 | 2.07 | 0.44 | 55.96 |
| CGAACAGG | | GTTCTGT | | | | | |
| CAGGAUGUUGUC | 250 | TCAAACCCGACA | 486 | 0.83 | 17.17 | 0.21 | 79.31 |
| GGGUUUGA | | ACATCCTG | | | | | |
| CGGCCUCAGCCU | 251 | TGCGGCAGAGGC | 487 | 0.93 | 6.71 | 0.40 | 60.06 |
| CUGCCGCA | | TGAGGCCG | | | | | |
| CAGCAGGAUGUU | 252 | AACCCGACAACA | 488 | 0.66 | 34.18 | 0.15 | 84.54 |
| GUCGGGUU | | TCCTGCTG | | | | | |
| GCAGAGAGAGGU | 253 | CAAGGAGCACCT | 489 | 0.83 | 17.29 | 0.14 | 85.95 |
| GCUCCUUG | | CTCTCTGC | | | | | |
| UCCAGUUCCAUG | 254 | CCCACACCCATG | 490 | 0.84 | 15.66 | 0.22 | 78.48 |
| GGUGUGGG | | GAACTGGA | | | | | |
| CCUCAGCCUGGC | 255 | TTCTTTCGGCCAG | 491 | 0.83 | 16.83 | 0.36 | 63.99 |
| CGAAAGAA | | GCTGAGG | | | | | |
| GGGCCUUUUAUU | 256 | CCCTCGCGAATA | 492 | 0.95 | 5.11 | 0.49 | 50.65 |
| CGCGAGGG | | AAAGGCCC | | | | | |
| GUCGGCCAGGCG | 257 | GCCACATCCGCCT | 493 | 0.85 | 15.35 | 0.25 | 74.59 |
| GAUGUGGC | | GGCCGAC | | | | | |
| GCUUGCUGCCUU | 258 | GGCCTGGGAAGG | 494 | 0.99 | 1.14 | 0.19 | 81.01 |
| CCCAGGCC | | CAGCAAGC | | | | | |
| GGUCCAGCAGGA | 259 | CGACAACATCCT | 495 | 0.68 | 31.78 | 0.20 | 79.93 |
| UGUUGUCG | | GCTGGACC | | | | | |
| CGGAGACCAUCC | 260 | CTCGACTGGGAT | 496 | 0.86 | 14.08 | 0.20 | 79.93 |
| CAGUCGAG | | GGTCTCCG | | | | | |
| UCUGCCUCGCGU | 261 | AGTCAACTACGC | 497 | 0.96 | 3.53 | 0.13 | 86.86 |
| AGUGACU | | GAGGCAGA | | | | | |
| AGGUAGUUCUCA | 262 | TTCCAGGATGAG | 498 | 0.93 | 7.36 | 0.37 | 62.62 |
| UCCUGGAA | | AACTACCT | | | | | |
| UCCUUGUAGUGG | 263 | AAGATCGTCCAC | 499 | 0.87 | 12.96 | 0.15 | 84.87 |
| ACGAUCUU | | TACAAGGA | | | | | |
| GCAUCCAAAACG | 264 | CCAATCCACGTTT | 500 | 0.97 | 2.54 | 0.27 | 72.69 |
| UGGAUUGG | | TGGATGC | | | | | |
| GUCCAGCAGGAU | 265 | CCGACAACATCC | 501 | 0.70 | 30.00 | 0.17 | 82.64 |
| GUGUCGG | | TGCTGGAC | | | | | |
| AGCUCCCGCAGC | 266 | GAGGTGACGCTG | 502 | 0.86 | 13.72 | 0.20 | 80.40 |
| GUCACCUC | | CGGGAGCT | | | | | |
| CGAGAGCAGCGC | 267 | CCTCACTTGCGCT | 503 | 1.02 | −2.19 | 0.63 | 37.11 |
| AAGUGAGG | | GCTCTCG | | | | | |
| CAGGGAAAGCGC | 268 | TATCGGAGGCGC | 504 | 0.89 | 11.10 | 0.08 | 91.59 |
| CUCCGAUA | | TTTCCCTG | | | | | |
| AUUUCAUAGGCG | 269 | GGTGTATTCGCCT | 505 | 1.05 | −4.54 | 0.56 | 44.15 |
| AAUACACC | | ATGAAAT | | | | | |
| UCGGCCAGGCGG | 270 | GGCCACATCCGC | 506 | 0.73 | 26.53 | 0.17 | 83.04 |
| AUGUGGCC | | CTGGCCGA | | | | | |
| AAGGGAUGUGUC | 271 | GACTTCCGGACA | 507 | 0.90 | 10.37 | 0.26 | 73.52 |
| CGGAAGUC | | CATCCCTT | | | | | |
| CUUGUAGUGGAC | 272 | GCAAGATCGTCC | 508 | 0.76 | 24.09 | 0.11 | 89.16 |
| GAUCUUGC | | ACTACAAG | | | | | |
| AGUCGGCCAGGC | 273 | CCACATCCGCCTG | 509 | 0.94 | 6.15 | 0.33 | 67.44 |
| GGAUGUGG | | GCCGACT | | | | | |
| GCCUCAGCCUGG | 274 | TCTTTCGGCCAGG | 510 | 1.05 | −4.82 | 0.37 | 63.11 |
| CCGAAAGA | | CTGAGGC | | | | | |
| AGCGUCACCUCG | 275 | GCTGAGGCCGAG | 511 | 0.78 | 22.10 | 0.35 | 64.70 |
| GCCUCAGC | | GTGACGCT | | | | | |
| CAGCGGCAGAGA | 276 | AGCACCTCTCTCT | 512 | 0.96 | 4.49 | 0.14 | 86.00 |
| GAGGUGCT | | GCCGCTG | | | | | |
| CCAGCGGCAGAG | 277 | GCACCTCTCTCTG | 513 | 0.97 | 3.23 | 0.15 | 84.55 |
| AGAGGUGC | | CCGCTGG | | | | | |
| UUGUAGUGGACG | 278 | GGCAAGATCGTC | 514 | 0.83 | 17.22 | 0.19 | 81.05 |
| AUCUUGCC | | CACTACAA | | | | | |
| AGGGAAAGCGCC | 279 | CTATCGGAGGCG | 515 | 1.01 | −1.12 | 0.25 | 75.50 |
| UCCGAUAG | | CTTTCCCT | | | | | |
| GGGAAAGCGCCU | 280 | CCTATCGGAGGC | 516 | 0.90 | 10.02 | 0.23 | 76.79 |
| CCGAUAGG | | GCTTTCCC | | | | | |

### Example 8: Selected Antisense Oligonucleotides Provided Dose-Dependent Reduction in DMPK Expression in Immortalized Myoblasts

Eighteen oligonucleotides from Example 7 were selected to be evaluated for their ability to reduce DMPK expression in a dose-responsive manner. DM1 C15 myoblasts were prepared as in Example 7 to yield differentiated myotubes in 96-well microplates. After seven days of differentiation, cells were transfected with individual oligonucleotides using Lipofectamine MessengerMax. Each oligonucleotide was tested in triplicate at concentrations of 0.046 nM, 0.137 nM, 0.412 nM, 1.235 nM, 3.704 nM, 11.11 nM, 33.33 nM, and 100 nM by 3-fold serial dilutions using 0.3 μL of Lipofectamine MessengerMax per well.

Following addition of oligonucleotide, cells were incubated for 72 hours prior to harvesting for total RNA. cDNA was synthesized from the total RNA extracts and qPCR was performed to determine expression levels of DMPK using a commercially available Taqman probe set in technical quadruplicate. All qPCR data were analyzed using a traditional AACT method and were normalized to a plate-based negative control that comprised of cells treated with vehicle control (0.3 μL/well Lipofectamine MessengerMax without any oligonucleotide). Data for each oligonucleotide to was fit to sigmoidal curve in order to determine an effective concentration of each oligonucleotide that provided a half-maximal response (EC-50). Results from these experiments are shown in Table 5.

Each of the eighteen antisense oligonucleotides selected for dose-dependent experimentation were capable of dose-dependently reducing DMPK in differentiated myotubes. Further, each of the tested antisense oligonucleotides reduced DMPK with EC-50 values below 25 nM. For example, antisense oligonucleotides comprising SEQ ID NOs: 161, 112, 119, 87, and 109 resulted in EC-50 values of 3.27 nM, 3.59 nM, 5.45 nM, 6.04 nM, and 24.59 nM, respectively. These data demonstrate that the antisense oligonucleotides shown in Table 5 are capable of dose-dependent reduction of DMPK in cellulo, suggesting that muscle-targeting complexes comprising these antisense oligonucleotides would be capable of targeting DMPK in muscle tissues in vivo.

TABLE 5
Ability of DMPK-targeting antisense oligonucleotides to reduce expression
of DMPK in dose-dependent manner in cellulo

| | | | | Results | |
|---|---|---|---|---|---|
| Antisense | SEQ | | SEQ | | Percent DMPK |
| Oligonucleotide | ID | DMPK Target | ID | | reduction |
| Sequence | NO: | Sequence | NO: | EC-50 (nM) | at 100 nM |
| GCAGGAUGUUGUCGGG | 98 | CAAACCCGACAA | 334 | 0.1679 | 89.77 |
| UUUG | | CATCCTGC | | | |
| AGCAGGAUGUUGUCGG | 115 | AAACCCGACAAC | 351 | 0.2266 | 85.81 |
| GUUU | | ATCCTGCT | | | |
| GCGUAGAAGGGCGUCU | 109 | GGGCAGACGCCC | 345 | 24.59 | 95.13 |
| GCCC | | TTCTACGC | | | |
| CCCAGCGCCCACCAGUC | 119 | TGTGACTGGTGG | 355 | 5.454 | 63.69 |
| ACA | | GCGCTGGG | | | |
| CCAUCUCGGCCGGAAUC | 77 | GCGGATTCCGGC | 313 | 0.44 | 95.42 |
| CGC | | CGAGATGG | | | |
| CGUUCCAUCUGCCCGCA | 93 | AGCTGCGGGCAG | 329 | 0.19 | 89.97 |
| GCU | | ATGGAACG | | | |
| CAGGGACAGCCGCUGG | 87 | AGTTCCAGCGGC | 323 | 6.04 | 90.59 |
| AACU | | TGTCCCTG | | | |
| CAUGGCAUACACCUGGC | 56 | CGGGCCAGGTGT | 292 | 0.42 | 75.28 |
| CCG | | ATGCCATG | | | |
| GCUUCAUCUUCACUACC | 85 | AGCGGTAGTGAA | 321 | 0.03 | 64.06 |
| GCU | | GATGAAGC | | | |
| GAAUGUCCGACAGUGU | 92 | GGAGACACTGTC | 328 | 0.07 | 97.23 |
| CUCC | | GGACATTC | | | |
| GGACGAUCUUGCCAUA | 112 | GACCTATGGCAA | 348 | 3.59 | 92.18 |
| GGUC | | GATCGTCC | | | |
| GCUGUCCCGGAGACCAU | 161 | GGGATGGTCTCC | 397 | 3.27 | 93.07 |
| CCC | | GGGACAGC | | | |
| GACAGAACAACGGCGA | 145 | CTGTTCGCCGTT | 381 | 0.08 | 94.32 |
| ACAG | | GTTCTGTC | | | |
| UGUUGUCGGGUUUGAU | 100 | GGACATCAAACC | 336 | 0.21 | 93.95 |
| GUCC | | CGACAACA | | | |
| CGAAUGUCCGACAGUG | 59 | GAGACACTGTCG | 295 | 0.18 | 95.93 |
| UCUC | | GACATTCG | | | |
| GGGCCUGGGACCUCACU | 69 | GACAGTGAGGTC | 305 | 0.07 | 90.58 |
| GUC | | CCAGGCCC | | | |
| CUCUGCCGCAGGGACAG | 71 | CGGCTGTCCCTG | 307 | 0.42 | 93.66 |
| CCG | | CGGCAGAG | | | |
| UUGCCAUAGGUCUCCGC | 79 | ACGGCGGAGAC | 315 | 0.37 | 93.70 |
| CGU | | CTATGGCAA | | | |

### Example 9: Targeting DMPK in Mouse Muscle Tissues with a Muscle-Targeting Complex

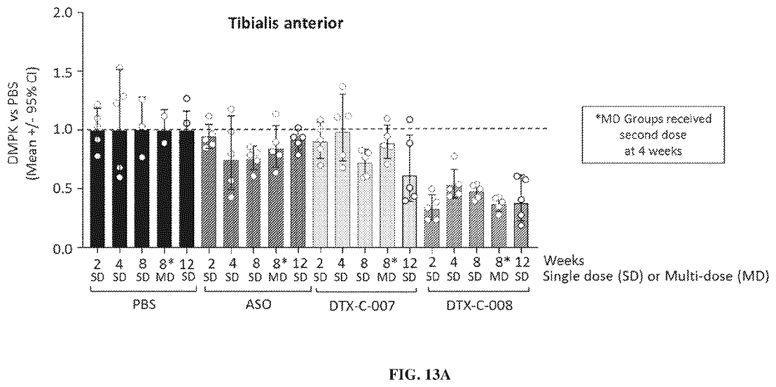

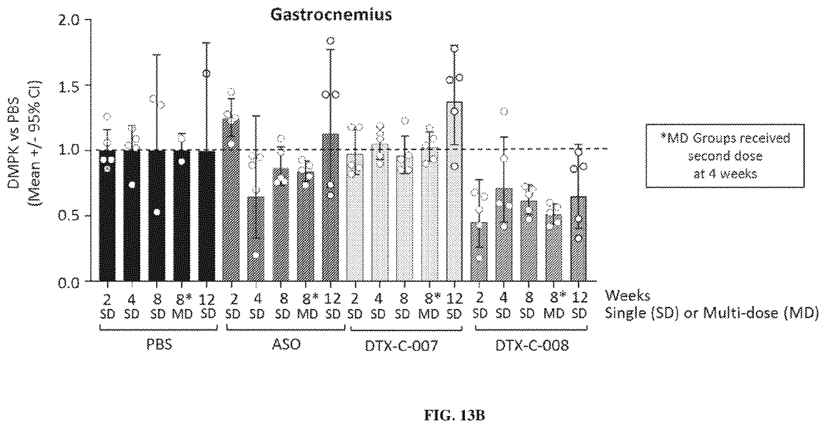

The muscle-targeting complex described in Example 2, DTX-C-008, was tested for time-dependent inhibition of DMPK in mouse tissues in vivo. C57BL/6 wild-type mice were intravenously injected with a single dose of a vehicle control (phosphate-buffered saline (PBS)), DTX-P-060 antisense oligonucleotide (ASO) (10 mg/kg of RNA), DTX-C-007 control complex (10 mg/kg of RNA), or DTX-C-008 (10 mg/kg of RNA) on Day 0 and euthanized after a prescribed period of time, as described in Table 6. One group of mice in each experimental condition was subjected to a second dose (multi-dose groups) at four weeks (Day 28). Following euthanization, the mice were segmented into isolated tissue types and samples of tibialis anterior and gastrocnemius muscle tissues were subsequently assayed for expression levels of DMPK (FIGS. 13A-13B).

TABLE 6
Experimental conditions

| | | Weeks after | |
|---|---|---|---|
| | | injection | |
| | | before | Number |
| Group | Dosage | euthanization | of mice |
| 1 | Single dose of vehicle (PBS) | 2 | 5 |
| 2 | Single dose of vehicle (PBS) | 4 | 5 |
| 3 | Single dose of vehicle (PBS) | 8 | 3 |
| 4 | Multi-dose of vehicle (PBS) | 8 | 2 |
| 5 | Single dose of vehicle (PBS) | 12 | 5 |
| 6 | Single dose of DTX-P-060 ASO | 2 | 5 |
| 7 | Single dose of DTX-P-060 ASO | 4 | 5 |
| 8 | Single dose of DTX-P-060 ASO | 8 | 5 |
| 9 | Multi-dose of DTX-P-060 ASO | 8 | 5 |
| 10 | Single dose of DTX-P-060 ASO | 12 | 5 |
| 11 | Single dose of DTX-C-007 control | 2 | 5 |
| 12 | Single dose of DTX-C-007 control | 4 | 5 |
| 13 | Single dose of DTX-C-007 control | 8 | 5 |
| 14 | Multi-dose of DTX-C-007 control | 8 | 5 |
| 15 | Single dose of DTX-C-007 control | 12 | 5 |
| 16 | Single dose of DTX-C-008 | 2 | 5 |
| 17 | Single dose of DTX-C-008 | 4 | 5 |
| 18 | Single dose of DTX-C-008 | 8 | 5 |
| 19 | Multi-dose of DTX-C-008 | 8 | 5 |
| 20 | Single dose of DTX-C-008 | 12 | 5 |

Mice treated with the DTX-C-008 complex demonstrated about 50-60% reduction in DMPK expression in tibialis anterior muscle (FIG. 13A) and about 30-50% reduction in DMPK expression in gastrocnemius muscle (FIG. 13B) for all of Groups 16-20 (2-12 weeks between injection and euthanization), relative to vehicle. These data show that a single dose of the muscle-targeting complex DTX-C-008 inhibits expression of DMPK for at least twelve weeks following administration of the complex.

In contrast, mice treated with the naked antisense oligonucleotide or the control complex did not demonstrate significant inhibition of DMPK expression across all experimental groups and tissues.

These data demonstrate that a muscle-targeting complex as described herein is capable of providing persistent inhibition of DMPK expression in vivo for up to 12 weeks following a single dose or administration of said muscle targeting complex.

### ADDITIONAL EMBODIMENTS

1. A method of treating a subject having an expansion of a disease-associated-repeat of a DMPK allele that is associated with myotonic dystrophy, the method comprising administering a muscle-targeting complex to the subject,
wherein the complex comprises a muscle-targeting agent covalently linked to a molecular payload configured for inhibiting expression or activity of a DMPK allele, and
wherein the administration results in inhibition of DMPK expression in muscle tissues by 40-60%, relative to a control, for a duration of time in the range of 4-8, 5-10, 8-12, 10-14, or 8-16 weeks following administration of the complex.
   2. A method of inhibiting DMPK expression in a subject, the method comprising administering a muscle-targeting complex to the subject,
wherein the complex comprises a muscle-targeting agent covalently linked to a molecular payload configured for inhibiting expression of a DMPK allele, and
wherein the administration results in inhibition of DMPK expression in muscle tissues by 40-60%, relative to a control, for a duration of time in the range of 4-8, 5-10, 8-12, 10-14, or 8-16 weeks following administration of the complex.
   3. The method of paragraph 1 or 2, wherein the molecular payload is an oligonucleotide.
   4. The method of paragraph 3, wherein the complex is administered at an oligonucleotide dose of 1-15 mg/kg.
   5. A complex comprising a muscle-targeting agent covalently linked to a molecular payload configured for inhibiting expression or activity of a DMPK allele comprising a disease-associated-repeat, wherein the muscle-targeting agent specifically binds to an internalizing cell surface receptor on muscle cells.
   6. The complex of paragraph 5, wherein the muscle-targeting agent is a muscle-targeting antibody.
   7. The complex of paragraph 6, wherein the muscle-targeting antibody specifically binds to an extracellular epitope of a transferrin receptor.
   8. The complex of paragraph 7, wherein the extracellular epitope of the transferrin receptor comprises an epitope of the apical domain of the transferrin receptor.
   9. The complex of paragraph 7 or 8, wherein the muscle-targeting antibody specifically binds to an epitope of a sequence in the range of C89 to F760 of SEQ ID NO: 1-3.
   10. The complex of any one of paragraphs 7 to 9, wherein the equilibrium dissociation constant (K_{d}) of binding of the muscle-targeting antibody to the transferrin receptor is in a range from 10⁻¹¹ M to 10⁻⁶ M.
   11. The complex of any one of paragraphs 7 to 10, wherein the muscle-targeting antibody competes for specific binding to an epitope of a transferrin receptor with an antibody listed in Table 1.
   12. The complex of paragraph 11, wherein the muscle-targeting antibody competes for specific binding to an epitope of a transferrin receptor with an K_{d} of less than or equal to 10⁻⁶ M.
   13. The complex of paragraph 12, wherein the K_{d} is in a range of 10⁻¹¹ M to 10⁻⁶ M.
   14. The complex of any one of paragraphs 7 to 13, wherein the muscle-targeting antibody does not specifically bind to the transferrin binding site of the transferrin receptor and/or wherein the muscle-targeting antibody does not inhibit binding of transferrin to the transferrin receptor.
   15. The complex of any one of paragraphs 7 to 14, wherein the muscle-targeting antibody is cross-reactive with extracellular epitopes of two or more of a human, non-human primate and rodent transferrin receptor.
   16. The complex of any one of paragraphs 7 to 15, wherein the complex is configured to promote transferrin receptor mediated internalization of the molecular payload into a muscle cell.
   17. The complex of any one of paragraphs 6 to 16, wherein the muscle-targeting antibody is a chimeric antibody, optionally wherein the chimeric antibody is a humanized monoclonal antibody.
   18. The complex of any one of paragraphs 6 to 17, wherein the muscle-targeting antibody is in the form of a ScFv, Fab fragment, Fab′ fragment, F(ab)₂ fragment, or Fv fragment.
   19. The complex of any one of paragraphs 5 to 18, wherein the molecular payload is an oligonucleotide.
   20. The complex of paragraph 19, wherein the oligonucleotide comprises at least 15 consecutive nucleotides of a sequence comprising any one of SEQ ID NO: 45-280.
   21. The complex of paragraph 20, wherein the oligonucleotide comprises a sequence comprising any one of SEQ ID NO: 45-280.
   22. The complex of paragraph 21, wherein the oligonucleotide comprises a sequence comprising any one of SEQ ID NO: 56, 59, 69, 71, 77, 79, 85, 87, 92, 93, 98, 100, 109, 112, 115, 119, 145, or 161.
   23. The complex of any one of paragraphs 19 to 22, wherein the oligonucleotide comprises a region of complementarity to any one of SEQ ID NO: 281-516.
   24. The complex of paragraph 23, wherein the oligonucleotide comprises a region of complementarity to at least 15 consecutive nucleotides of any one of SEQ ID NO: 281-516.
   25. The complex of any one of paragraphs 19 to 24, wherein the oligonucleotide comprises a region of complementarity to the DMPK allele comprising the disease-associated-repeat expansion.
   26. The complex of any one of paragraphs 5 to 18, wherein the molecular payload is a polypeptide.
   27. The complex of paragraph 26, wherein the polypeptide is a muscleblind-like (MBNL) polypeptide.
   28. The complex of any one of paragraphs 19 to 25, wherein the oligonucleotide comprises an antisense strand that hybridizes, in a cell, with a wild-type DMPK mRNA transcript encoded by the allele, wherein the DMPK mRNA transcript comprises repeating units of a CUG trinucleotide sequence.
   29. The complex of any one of paragraphs 19 to 25, wherein the oligonucleotide comprises an antisense strand that hybridizes, in a cell, with a mutant DMPK mRNA transcript encoded by the allele, wherein the DMPK mRNA transcript comprises repeating units of a CUG trinucleotide sequence.
   30. The complex of any one of paragraphs 5 to 29, wherein the disease-associated-repeat is 38 to 200 repeating units in length.
   31. The complex of paragraph 30, wherein the disease-associated-repeat is associated with late onset myotonic dystrophy.
   32. The complex of any one of paragraphs 5 to 29, wherein the disease-associated-repeat is 100 to 10,000 repeat units in length.
   33. The complex of paragraph 32, wherein the disease-associated-repeat is associated with congenital myotonic dystrophy.
   34. The complex of any one of paragraphs 19 to 25 and 28 to 33, wherein the oligonucleotide comprises at least one modified internucleotide linkage.
   35. The complex of paragraph 34, wherein the at least one modified internucleotide linkage is a phosphorothioate linkage 36. The complex of paragraph 35, wherein the oligonucleotide comprises phosphorothioate linkages in the Rp stereochemical conformation and/or in the Sp stereochemical conformation.
   37. The complex of paragraph 36, wherein the oligonucleotide comprises phosphorothioate linkages that are all in the Rp stereochemical conformation or that are all in the Sp stereochemical conformation.
   38. The complex of any one of paragraphs 19 to 25 and 28 to 37, wherein the oligonucleotide comprises one or more modified nucleotides.
   39. The complex of paragraph 38, wherein the one or more modified nucleotides are 2′-modified nucleotides.
   40. The complex of any one of paragraphs 19 to 25 and 28 to 39, wherein the oligonucleotide is a gapmer oligonucleotide that directs RNAse H-mediated cleavage of a DMPK mRNA transcript in a cell.
   41. The complex of paragraph 40, wherein the gapmer oligonucleotide comprises a central portion of 5 to 15 deoxyribonucleotides flanked by wings of 2 to 8 modified nucleotides.
   42. The complex of paragraph 41, wherein the modified nucleotides of the wings are 2′-modified nucleotides.
   43. The complex of any one of paragraphs 19 to 25 and 28 to 39, wherein the oligonucleotide is a mixmer oligonucleotide.
   44. The complex of paragraph 43, wherein the mixmer oligonucleotide inhibits binding of muscleblind-like protein 1, muscleblind-like protein 2, or muscleblind-like protein 3 to the DMPK mRNA transcript.
   45. The complex of paragraph 43 or 44, wherein the mixmer oligonucleotide comprises two or more different 2′ modified nucleotides.
   46. The complex of any one of paragraphs 19 or 25 and 28 to 39, wherein the oligonucleotide is an RNAi oligonucleotide that promotes RNAi-mediated cleavage of the DMPK mRNA transcript.
   47. The complex of paragraph 46, wherein the RNAi oligonucleotide is a double-stranded oligonucleotide of 19 to 25 nucleotides in length.
   48. The complex of paragraph 46 or 47, wherein the RNAi oligonucleotide comprises at least one 2′ modified nucleotide.
   49. The complex of any one of paragraphs 39, 42, 45, or 48, wherein each 2′ modified nucleotide is selected from the group consisting of: 2′-O-methyl, 2′-fluoro (2′-F), 2′-O-methoxyethyl (2′-MOE), and 2′, 4′-bridged nucleotides.
   50. The complex of paragraph 38, wherein the one or more modified nucleotides are bridged nucleotides.
   51. The complex of any one of paragraph 39, 42, 45, or 48, wherein at least one 2′ modified nucleotide is a 2′,4′-bridged nucleotide selected from: 2′,4′-constrained 2′-O-ethyl (cEt) and locked nucleic acid (LNA) nucleotides.
   52. The complex of any one of paragraphs 19 to 25 and 28 to 39, wherein the oligonucleotide comprises a guide sequence for a genome editing nuclease.
   53. The complex of any one of paragraphs 19 to 25 and 28 to 39, wherein the oligonucleotide is phosphorodiamidite morpholino oligomer.
   54. The complex of any one of paragraphs 5 to 53, wherein the muscle-targeting agent is covalently linked to the molecular payload via a cleavable linker.
   55. The complex of paragraph 54, wherein the cleavable linker is selected from: a protease-sensitive linker, pH-sensitive linker, and glutathione-sensitive linker.
   56. The complex of paragraph 55, wherein the cleavable linker is a protease-sensitive linker.
   57. The complex of paragraph 56, wherein the protease-sensitive linker comprises a sequence cleavable by a lysosomal protease and/or an endosomal protease.
   58. The complex of paragraph 56, wherein the protease-sensitive linker comprises a valine-citrulline dipeptide sequence.
   59. The complex of paragraph 55, wherein the linker is pH-sensitive linker that is cleaved at a pH in a range of 4 to 6.
   60. The complex of any one of paragraphs 5 to 53, wherein the muscle-targeting agent is covalently linked to the molecular payload via a non-cleavable linker.
   61. The complex of paragraph 60, wherein the non-cleavable linker is an alkane linker.
   62. The complex of any of paragraphs 6 to 61, wherein the muscle-targeting antibody comprises a non-natural amino acid to which the oligonucleotide is covalently linked.
   63. The complex of any of paragraphs 6 to 61, wherein the muscle-targeting antibody is covalently linked to the oligonucleotide via conjugation to a lysine residue or a cysteine residue of the antibody.
   64. The complex of paragraph 63, wherein the muscle-targeting antibody is conjugated to the cysteine via a maleimide-containing linker, optionally wherein the maleimide-containing linker comprises a maleimidocaproyl or maleimidomethyl cyclohexane-1-carboxylate group.
   65. The complex of any one of paragraphs 6 to 64, wherein the muscle-targeting antibody is a glycosylated antibody that comprises at least one sugar moiety to which the oligonucleotide is covalently linked.
   66. The complex of paragraph 65, wherein the sugar moiety is a branched mannose.
   67. The complex of paragraph 65 or 66, wherein the muscle-targeting antibody is a glycosylated antibody that comprises one to four sugar moieties each of which is covalently linked to a separate oligonucleotide.
   68. The complex of paragraph 65, wherein the muscle-targeting antibody is a fully-glycosylated antibody.
   69. The complex of paragraph 65, wherein the muscle-targeting antibody is a partially-glycosylated antibody.
   70. The complex of paragraph 69, wherein the partially-glycosylated antibody is produced via chemical or enzymatic means.
   71. The complex of paragraph 69, wherein the partially-glycosylated antibody is produced in a cell, cell that is deficient for an enzyme in the N- or O-glycosylation pathway.
   72. A method of delivering a molecular payload to a cell expressing transferrin receptor, the method comprising contacting the cell with the complex of any one of paragraphs 5 to 71.
   73. A method of inhibiting activity of DMPK in a cell, the method comprising contacting the cell with the complex of any one of paragraphs 5 to 71 in an amount effective for promoting internalization of the molecular payload to the cell.
   74. The method of paragraph 73, wherein the cell is in vitro.
   75. The method of paragraph 73, wherein the cell is in a subject.
   76. The method of paragraph 75, wherein the subject is a human.
   77. The method of any one of paragraphs 73 to 76, wherein the complex inhibits the expression of DMPK.
   78. The method of any one of paragraphs 73 to 77, wherein the cell is contacted with a single dose of the complex.
   79. The method of paragraph 78, wherein a single dose of the complex inhibits the expression of DMPK for at least two, four, eight, or twelve weeks.
   80. The method of paragraph 79, wherein the complex inhibits the expression of DMPK by at least 30%, 40%, 50%, or 60% relative to a control.
   81. The method of paragraph 79 or 80, the complex inhibits the expression of DMPK in muscle tissues by 40-60% for at least 12 weeks following administration of the single dose, relative to a control
   82. A method of treating a subject having an expansion of a disease-associated-repeat of a DMPK allele that is associated with myotonic dystrophy, the method comprising administering to the subject an effective amount of the complex of any one of paragraphs 5 to 71.
   83. The method of paragraph 82, wherein the disease-associated-repeat comprises repeating units of a trinucleotide sequence.
   84. The method of paragraph 82, wherein the trinucleotide sequence is a CTG trinucleotide sequence.
   85. The method of any one of paragraphs 82 to 84, wherein the disease-associated-repeat is 38 to 200 repeating units in length.
   86. The method of 85, wherein the disease-associated-repeat is associated with late onset myotonic dystrophy.
   87. The method of any one of paragraphs 82 to 84, wherein the disease-associated-repeat is 100 to 10,000 repeating units in length.
   88. The method of 87, wherein the disease-associated-repeat is associated with congenital myotonic dystrophy.
   89. The method of any one of paragraphs 82 to 88, wherein administration of the complex results in inhibition of the expression of DMPK in muscle tissues.
   90. The method of any one of paragraphs 82 to 89, wherein the complex is intravenously administered to the subject.
   91. The method of any one of paragraphs 82 to 90, wherein an effective amount of the complex is a molecular payload dose of 1-15 mg/kg.
   92. The method of any one of paragraphs 82 to 91, wherein the complex is administered to the subject in a single dose.
   93. The method of paragraph 92, wherein administration of a single dose of the complex results in inhibition of the expression of DMPK in muscle tissues for at least two, four, eight, or twelve weeks.
   94. The method of paragraph 93, wherein the administration of a single dose of the complex results in inhibition of the expression of DMPK in muscle tissues by at least 30%, 40%, 50%, or 60% relative to a control.
   95. The method of paragraph 93 or 94, wherein the administration of a single dose of the complex results in inhibition of the expression of DMPK in muscle tissues for at least 12 weeks following administration of the single dose.
   96. The method of paragraph 95, wherein the administration of a single dose of the complex results in inhibition of the expression of DMPK in muscle tissues by 40-60%, relative to a control, for at least 12 weeks following administration of the single dose.
   97. The method of paragraph 93 or 94, wherein the administration of a single dose of the complex results in inhibition of the expression of DMPK in muscle tissues for a duration of time in the range of 4-8, 5-10, 8-12, 10-14, or 8-16 weeks following administration of the single dose.
   98. The method of paragraph 97, wherein the administration of a single dose of the complex results in inhibition of the expression of DMPK in muscle tissues by 40-60%, relative to a control, for a duration of time in the range of 4-8, 5-10, 8-12, 10-14, or 8-16 weeks following administration of the single dose.
   99. The method of paragraph 93 or 94, wherein the administration of a single dose of the complex results in inhibition of the expression of DMPK in muscle tissues by 40-60%, relative to a control, at 12 weeks following administration of the single dose.
   100. The method of any one of paragraphs 82 to 91, wherein the complex is administered to the subject in a single dose once every 4-8, 5-10, 8-12, or 8-16 weeks.
   101. The method of paragraph 100, wherein the complex is administered to the subject in a single dose once every 12 weeks.
   102. The method of any one of paragraphs 92 to 101, wherein the single dose is a molecular payload dose of 1-15 mg/kg.
   103. The method of paragraph 102, wherein the single dose is a molecular payload dose of 10 mg/kg.

### EQUIVALENTS AND SCOPE

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A method of treating a subject having an expansion of a disease-associated-repeat of a DMPK allele that is associated with myotonic dystrophy, the method comprising administering a muscle-targeting complex to the subject,
wherein the complex comprises a muscle-targeting agent covalently linked to a molecular payload configured for inhibiting expression or activity of a DMPK allele, and wherein the administration results in inhibition of DMPK expression in muscle tissues by 40-60%, relative to a control, for a duration of time in the range of 4-8, 5-10, 8-12, 10-14, or 8-16 weeks following administration of the complex.

2. A method of inhibiting DMPK expression in a subject, the method comprising administering a muscle-targeting complex to the subject,
wherein the complex comprises a muscle-targeting agent covalently linked to a molecular payload configured for inhibiting expression of a DMPK allele, and
wherein the administration results in inhibition of DMPK expression in muscle tissues by 40-60%, relative to a control, for a duration of time in the range of 4-8, 5-10, 8-12, 10-14, or 8-16 weeks following administration of the complex.

3. The method of claim 1 or claim 2, wherein the complex is administered at a molecular payload dose of 1-15 mg/kg, optionally wherein the complex is administered at a molecular payload dose of 10 mg/kg.

4. The method of any one of claims 1-3, wherein the complex is administered to the subject in a single dose.

5. The method of any one of claims 1-4, wherein the muscle-targeting agent is a muscle-targeting antibody.

6. The method of claim 5, wherein the muscle-targeting antibody is a transferrin receptor antibody, optionally wherein the muscle-targeting antibody binds to an epitope of a sequence in the range of C89 to F760 of SEQ ID NO: 1-3.

7-23. (canceled)


SEQUENCE LISTING (516 sequences)

SEQ ID NO: 1 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 3 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 4 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 5 - protein, 197 aa, Artificial Sequence
  Synthetic polypeptide
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 6 - protein, 7 aa, Artificial Sequence
  Synthetic polypeptide
ASSLNIA

SEQ ID NO: 7 - protein, 12 aa, Artificial Sequence
  Synthetic polypeptide
SKTFNTHPQSTP

SEQ ID NO: 8 - protein, 12 aa, Artificial Sequence
  Synthetic polypeptide
TARGEHKEEELI

SEQ ID NO: 9 - protein, 9 aa, Artificial Sequence
  Synthetic polypeptide
CQAQGQLVC

SEQ ID NO: 10 - protein, 9 aa, Artificial Sequence
  Synthetic polypeptide
CSERSMNFC

SEQ ID NO: 11 - protein, 9 aa, Artificial Sequence
  Synthetic polypeptide
CPKTRRVPC

SEQ ID NO: 12 - protein, 20 aa, Artificial Sequence
  Synthetic polypeptide
WLSEAGPVVTVRALRGTGSW

SEQ ID NO: 13 - protein, 9 aa, Artificial Sequence
  Synthetic polypeptide
CMQHSMRVC

SEQ ID NO: 14 - protein, 7 aa, Artificial Sequence
  Synthetic polypeptide
DDTRHWG

SEQ ID NO: 15 - DNA, 2859 bp, Homo sapiens
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 16 - DNA, 2683 bp, Mus musculus
gaactggccagagagacccaagggatagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcgccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaa

SEQ ID NO: 17 - protein, 5 aa, Artificial Sequence
  Synthetic polypeptide
SYWMH

SEQ ID NO: 18 - protein, 17 aa, Artificial Sequence
  Synthetic polypeptide
EINPTNGRTNYIEKFKS

SEQ ID NO: 19 - protein, 7 aa, Artificial Sequence
  Synthetic polypeptide
GTRAYHY

SEQ ID NO: 20 - protein, 11 aa, Artificial Sequence
  Synthetic polypeptide
RASDNLYSNLA

SEQ ID NO: 21 - protein, 7 aa, Artificial Sequence
  Synthetic polypeptide
DATNLAD

SEQ ID NO: 22 - protein, 9 aa, Artificial Sequence
  Synthetic polypeptide
QHFWGTPLT

SEQ ID NO: 23 - protein, 7 aa, Artificial Sequence
  Synthetic polypeptide
GYTFTSY

SEQ ID NO: 24 - protein, 6 aa, Artificial Sequence
  Synthetic polypeptide
NPTNGR

SEQ ID NO: 25 - protein, 6 aa, Artificial Sequence
  Synthetic polypeptide
TSYWMH

SEQ ID NO: 26 - protein, 13 aa, Artificial Sequence
  Synthetic polypeptide
WIGEINPTNGRTN

SEQ ID NO: 27 - protein, 6 aa, Artificial Sequence
  Synthetic polypeptide
ARGTRA

SEQ ID NO: 28 - protein, 7 aa, Artificial Sequence
  Synthetic polypeptide
YSNLAWY

SEQ ID NO: 29 - protein, 10 aa, Artificial Sequence
  Synthetic polypeptide
LLVYDATNLA

SEQ ID NO: 30 - protein, 8 aa, Artificial Sequence
  Synthetic polypeptide
QHFWGTPL

SEQ ID NO: 31 - protein, 9 aa, Artificial Sequence
  Synthetic polypeptide
QHFAGTPLT

SEQ ID NO: 32 - protein, 8 aa, Artificial Sequence
  Synthetic polypeptide
QHFAGTPL

SEQ ID NO: 33 - protein, 116 aa, Artificial Sequence
  Synthetic polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 34 - protein, 107 aa, Artificial Sequence
  Synthetic polypeptide
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 35 - protein, 116 aa, Artificial Sequence
  Synthetic polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 36 - protein, 107 aa, Artificial Sequence
  Synthetic polypeptide
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 37 - protein, 330 aa, Artificial Sequence [sequence omitted]

SEQ ID NO: 38 - protein, 107 aa, Artificial Sequence [sequence omitted]

SEQ ID NO: 39 - protein, 446 aa, Artificial Sequence
  Synthetic polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, Artificial Sequence
  Synthetic polypeptide
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 446 aa, Artificial Sequence
  Synthetic polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 214 aa, Artificial Sequence
  Synthetic polypeptide
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 226 aa, Artificial Sequence
  Synthetic polypeptide
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 44 - protein, 226 aa, Artificial Sequence
  Synthetic polypeptide
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 45 - RNA, 20 nt, Artificial Sequence
  Synthetic Polynucleotide
ggacggcccggcuugcugcc

SEQ ID NO: 46 - RNA, 20 nt, Artificial Sequence
  Synthetic Polynucleotide
gggcccggaucacaggacug

SEQ ID NO: 47 - RNA, 20 nt, Artificial Sequence
  Synthetic Polynucleotide
caaacuugcucagcaguguc

SEQ ID NO: 48 - RNA, 20 nt, Artificial Sequence
  Synthetic Polynucleotide
aaacuugcucagcaguguca

SEQ ID NO: 49 - RNA, 20 nt, Artificial Sequence
  Synthetic Polynucleotide
cggauggccuccaucucccg

SEQ ID NO: 50 - RNA, 20 nt, Artificial Sequence
  Synthetic Polynucleotide
cucggccggaauccgcuccc

SEQ ID NO: 51 - RNA, 20 nt, Artificial Sequence
  Synthetic Polynucleotide
ucucggccggaauccgcucc

SEQ ID NO: 52 - RNA, 20 nt, Artificial Sequence
  Synthetic Polynucleotide
ugcucagcagugucagcagg

SEQ ID NO: 53 - RNA, 20 nt, Artificial Sequence
  Synthetic Polynucleotide
uugucggguuugaugucccu

SEQ ID NO: 54 - RNA, 20 nt, Artificial Sequence
  Synthetic Polynucleotide
guugucggguuugauguccc

SEQ ID NO: 55 - RNA, 20 nt, Artificial Sequence
  Synthetic Polynucleotide
uccgccagguagaagcgcgc

SEQ ID NO: 56 - RNA, 20 nt, Artificial Sequence
  Synthetic Polynucleotide
cauggcauacaccuggcccg

SEQ ID NO: 57 - RNA, 20 nt, Artificial Sequence
  Synthetic Polynucleotide
aacuugcucagcagugucag

SEQ ID NO: 58 - RNA, 20 nt, Artificial Sequence
  Synthetic Polynucleotide
cagcugcgugauccaccgcc

SEQ ID NO: 59 - RNA, 20 nt, Artificial Sequence
  Synthetic Polynucleotide
cgaauguccgacagugucuc

SEQ ID NO: 60 - RNA, 20 nt, Artificial Sequence
  Synthetic Polynucleotide
gaagucggccaggcggaugu

SEQ ID NO: 61 - RNA, 20 nt, Artificial Sequence
  Synthetic Polynucleotide
ugucggguuugaugucccug

SEQ ID NO: 62 - RNA, 20 nt, Artificial Sequence
  Synthetic Polynucleotide
ggauggccuccaucucccgg

SEQ ID NO: 63 - RNA, 20 nt, Artificial Sequence
  Synthetic Polynucleotide
aggauguugucggguuugau

SEQ ID NO: 64 - RNA, 20 nt, Artificial Sequence
  Synthetic Polynucleotide
gucggguuugaugucccugu

SEQ ID NO: 65 - RNA, 20 nt, Artificial Sequence
  Synthetic Polynucleotide
aauacuccaugaccagguac

SEQ ID NO: 66 - RNA, 20 nt, Artificial Sequence
  Synthetic Polynucleotide
cuuguucaugaucuucaugg

SEQ ID NO: 67 - RNA, 20 nt, Artificial Sequence
  Synthetic Polynucleotide
ucagugcauccaaaacgugg

SEQ ID NO: 68 - RNA, 20 nt, Artificial Sequence
  Synthetic Polynucleotide
cugucccggagaccauccca

SEQ ID NO: 69 - RNA, 20 nt, Artificial Sequence
  Synthetic Polynucleotide
gggccugggaccucacuguc

SEQ ID NO: 70 - RNA, 20 nt, Artificial Sequence
  Synthetic Polynucleotide
cccacguaauacuccaugac

SEQ ID NO: 71 - RNA, 20 nt, Artificial Sequence
  Synthetic Polynucleotide
cucugccgcagggacagccg

SEQ ID NO: 72 - RNA, 20 nt, Artificial Sequence
  Synthetic Polynucleotide
cugugcacguagccaagccg

SEQ ID NO: 73 - RNA, 20 nt, Artificial Sequence
  Synthetic Polynucleotide
ugcccauccacgucagggcc

SEQ ID NO: 74 - RNA, 20 nt, Artificial Sequence
  Synthetic Polynucleotide
agcgccuccgauaggccagg

SEQ ID NO: 75 - RNA, 20 nt, Artificial Sequence
  Synthetic Polynucleotide
ugugcacguagccaagccgg

SEQ ID NO: 76 - RNA, 20 nt, Artificial Sequence
  Synthetic Polynucleotide
gaccagguacagguaguucu

SEQ ID NO: 77 - RNA, 20 nt, Artificial Sequence
  Synthetic Polynucleotide
ccaucucggccggaauccgc

SEQ ID NO: 78 - RNA, 20 nt, Artificial Sequence
  Synthetic Polynucleotide
caucucggccggaauccgcu

SEQ ID NO: 79 - RNA, 20 nt, Artificial Sequence
  Synthetic Polynucleotide
uugccauaggucuccgccgu

SEQ ID NO: 80 - RNA, 20 nt, Artificial Sequence
  Synthetic Polynucleotide
acagcgguccagcaggaugu

SEQ ID NO: 81 - RNA, 20 nt, Artificial Sequence
  Synthetic Polynucleotide
aaagcgccuccgauaggcca

SEQ ID NO: 82 - RNA, 20 nt, Artificial Sequence
  Synthetic Polynucleotide
gccaaagaagaagggaugug

SEQ ID NO: 83 - RNA, 20 nt, Artificial Sequence
  Synthetic Polynucleotide
cacguaauacuccaugacca

SEQ ID NO: 84 - RNA, 20 nt, Artificial Sequence
  Synthetic Polynucleotide
aucucggccggaauccgcuc

SEQ ID NO: 85 - RNA, 20 nt, Artificial Sequence
  Synthetic Polynucleotide
gcuucaucuucacuaccgcu

SEQ ID NO: 86 - RNA, 20 nt, Artificial Sequence
  Synthetic Polynucleotide
gccaucucggccggaauccg

SEQ ID NO: 87 - RNA, 20 nt, Artificial Sequence
  Synthetic Polynucleotide
cagggacagccgcuggaacu

SEQ ID NO: 88 - RNA, 20 nt, Artificial Sequence
  Synthetic Polynucleotide
augacaaucuccgccaggua

SEQ ID NO: 89 - RNA, 20 nt, Artificial Sequence
  Synthetic Polynucleotide
ggccaugacaaucuccgcca

SEQ ID NO: 90 - RNA, 20 nt, Artificial Sequence
  Synthetic Polynucleotide
auacuccaugaccagguaca

SEQ ID NO: 91 - RNA, 20 nt, Artificial Sequence
  Synthetic Polynucleotide
gccucugccucgcguaguug

SEQ ID NO: 92 - RNA, 20 nt, Artificial Sequence
  Synthetic Polynucleotide
gaauguccgacagugucucc

SEQ ID NO: 93 - RNA, 20 nt, Artificial Sequence
  Synthetic Polynucleotide
cguuccaucugcccgcagcu

SEQ ID NO: 94 - RNA, 20 nt, Artificial Sequence
  Synthetic Polynucleotide
ccuuguaguggacgaucuug

SEQ ID NO: 95 - RNA, 20 nt, Artificial Sequence
  Synthetic Polynucleotide
aucuccgccagguagaagcg

SEQ ID NO: 96 - RNA, 20 nt, Artificial Sequence
  Synthetic Polynucleotide
cucaggcucugccgggugag

SEQ ID NO: 97 - RNA, 20 nt, Artificial Sequence
  Synthetic Polynucleotide
ugcuucaucuucacuaccgc

SEQ ID NO: 98 - RNA, 20 nt, Artificial Sequence
  Synthetic Polynucleotide
gcaggauguugucggguuug

SEQ ID NO: 99 - RNA, 20 nt, Artificial Sequence
  Synthetic Polynucleotide
ggccucagccucugccgcag

SEQ ID NO: 100 - RNA, 20 nt, Artificial Sequence
  Synthetic Polynucleotide
uguugucggguuugaugucc

SEQ ID NO: 101 - RNA, 20 nt, Artificial Sequence
  Synthetic Polynucleotide
ccacguaauacuccaugacc

SEQ ID NO: 102 - RNA, 20 nt, Artificial Sequence
  Synthetic Polynucleotide
ccguuccaucugcccgcagc

SEQ ID NO: 103 - RNA, 20 nt, Artificial Sequence
  Synthetic Polynucleotide
uucccgaguaagcaggcaga

SEQ ID NO: 104 - RNA, 20 nt, Artificial Sequence
  Synthetic Polynucleotide
ugaucuucauggcauacacc

SEQ ID NO: 105 - RNA, 20 nt, Artificial Sequence
  Synthetic Polynucleotide
agggacagccgcuggaacug

SEQ ID NO: 106 - RNA, 20 nt, Artificial Sequence
  Synthetic Polynucleotide
ggguuugaugucccugugca

SEQ ID NO: 107 - RNA, 20 nt, Artificial Sequence
  Synthetic Polynucleotide
ugacaaucuccgccagguag

SEQ ID NO: 108 - RNA, 20 nt, Artificial Sequence
  Synthetic Polynucleotide
cacagcgguccagcaggaug

SEQ ID NO: 109 - RNA, 20 nt, Artificial Sequence
  Synthetic Polynucleotide
gcguagaagggcgucugccc

SEQ ID NO: 110 - RNA, 20 nt, Artificial Sequence
  Synthetic Polynucleotide
cucagccucugccgcaggga

SEQ ID NO: 111 - RNA, 20 nt, Artificial Sequence
  Synthetic Polynucleotide
gucucagugcauccaaaacg

SEQ ID NO: 112 - RNA, 20 nt, Artificial Sequence
  Synthetic Polynucleotide
ggacgaucuugccauagguc

SEQ ID NO: 113 - RNA, 20 nt, Artificial Sequence
  Synthetic Polynucleotide
ucagcagugucagcaggucc

SEQ ID NO: 114 - RNA, 20 nt, Artificial Sequence
  Synthetic Polynucleotide
gcuccugggcggcgccagac

SEQ ID NO: 115 - RNA, 20 nt, Artificial Sequence
  Synthetic Polynucleotide
agcaggauguugucggguuu

SEQ ID NO: 116 - RNA, 20 nt, Artificial Sequence
  Synthetic Polynucleotide
auccgcuccugcaacugccg

SEQ ID NO: 117 - RNA, 20 nt, Artificial Sequence
  Synthetic Polynucleotide
aggagcagggaaagcgccuc

SEQ ID NO: 118 - RNA, 20 nt, Artificial Sequence
  Synthetic Polynucleotide
acaccuggcccgucugcuuc

SEQ ID NO: 119 - RNA, 20 nt, Artificial Sequence
  Synthetic Polynucleotide
cccagcgcccaccagucaca

SEQ ID NO: 120 - RNA, 20 nt, Artificial Sequence
  Synthetic Polynucleotide
gcucccucugccugcagcaa

SEQ ID NO: 121 - RNA, 20 nt, Artificial Sequence
  Synthetic Polynucleotide
gcucaggcucugccggguga

SEQ ID NO: 122 - RNA, 20 nt, Artificial Sequence
  Synthetic Polynucleotide
uugaugucccugugcacgua

SEQ ID NO: 123 - RNA, 20 nt, Artificial Sequence
  Synthetic Polynucleotide
gccucagccucugccgcagg

SEQ ID NO: 124 - RNA, 20 nt, Artificial Sequence
  Synthetic Polynucleotide
gguaguucucauccuggaag

SEQ ID NO: 125 - RNA, 20 nt, Artificial Sequence
  Synthetic Polynucleotide
cagcgcccaccagucacacu

SEQ ID NO: 126 - RNA, 20 nt, Artificial Sequence
  Synthetic Polynucleotide
cccaaacuugcucagcagug

SEQ ID NO: 127 - RNA, 20 nt, Artificial Sequence
  Synthetic Polynucleotide
cuugccauaggucuccgccg

SEQ ID NO: 128 - RNA, 20 nt, Artificial Sequence
  Synthetic Polynucleotide
uacaccuggcccgucugcuu

SEQ ID NO: 129 - RNA, 20 nt, Artificial Sequence
  Synthetic Polynucleotide
ccagcgcccaccagucacac

SEQ ID NO: 130 - RNA, 20 nt, Artificial Sequence
  Synthetic Polynucleotide
ggccucagccuggccgaaag

SEQ ID NO: 131 - RNA, 20 nt, Artificial Sequence
  Synthetic Polynucleotide
aaucuccgccagguagaagc

SEQ ID NO: 132 - RNA, 20 nt, Artificial Sequence
  Synthetic Polynucleotide
auggcauacaccuggcccgu

SEQ ID NO: 133 - RNA, 20 nt, Artificial Sequence
  Synthetic Polynucleotide
ccaugacaaucuccgccagg

SEQ ID NO: 134 - RNA, 20 nt, Artificial Sequence
  Synthetic Polynucleotide
uccccaaacuugcucagcag

SEQ ID NO: 135 - RNA, 20 nt, Artificial Sequence
  Synthetic Polynucleotide
gauguugucggguuugaugu

SEQ ID NO: 136 - RNA, 20 nt, Artificial Sequence
  Synthetic Polynucleotide
guuugcccauccacgucagg

SEQ ID NO: 137 - RNA, 20 nt, Artificial Sequence
  Synthetic Polynucleotide
cggacggcccggcuugcugc

SEQ ID NO: 138 - RNA, 20 nt, Artificial Sequence
  Synthetic Polynucleotide
cuccgccagguagaagcgcg

SEQ ID NO: 139 - RNA, 20 nt, Artificial Sequence
  Synthetic Polynucleotide
guacagguaguucucauccu

SEQ ID NO: 140 - RNA, 20 nt, Artificial Sequence
  Synthetic Polynucleotide
agggcgucugcccauagaac

SEQ ID NO: 141 - RNA, 20 nt, Artificial Sequence
  Synthetic Polynucleotide
uggccacagcgguccagcag

SEQ ID NO: 142 - RNA, 20 nt, Artificial Sequence
  Synthetic Polynucleotide
cguaguugacuggcgaaguu

SEQ ID NO: 143 - RNA, 20 nt, Artificial Sequence
  Synthetic Polynucleotide
ucugccgcagggacagccgc

SEQ ID NO: 144 - RNA, 20 nt, Artificial Sequence
  Synthetic Polynucleotide
aagcgccuccgauaggccag

SEQ ID NO: 145 - RNA, 20 nt, Artificial Sequence
  Synthetic Polynucleotide
gacagaacaacggcgaacag

SEQ ID NO: 146 - RNA, 20 nt, Artificial Sequence
  Synthetic Polynucleotide
gcucagcagugucagcaggu

SEQ ID NO: 147 - RNA, 20 nt, Artificial Sequence
  Synthetic Polynucleotide
augaucuucauggcauacac

SEQ ID NO: 148 - RNA, 20 nt, Artificial Sequence
  Synthetic Polynucleotide
uuugcccauccacgucaggg

SEQ ID NO: 149 - RNA, 20 nt, Artificial Sequence
  Synthetic Polynucleotide
acuugcucagcagugucagc

SEQ ID NO: 150 - RNA, 20 nt, Artificial Sequence
  Synthetic Polynucleotide
ugaugucccugugcacguag

SEQ ID NO: 151 - RNA, 20 nt, Artificial Sequence
  Synthetic Polynucleotide
aaauaccgaggaaugucggg

SEQ ID NO: 152 - RNA, 20 nt, Artificial Sequence
  Synthetic Polynucleotide
ggcgaauacacccagcgccc

SEQ ID NO: 153 - RNA, 20 nt, Artificial Sequence
  Synthetic Polynucleotide
agacaauaaauaccgaggaa

SEQ ID NO: 154 - RNA, 20 nt, Artificial Sequence
  Synthetic Polynucleotide
cccgucugcuucaucuucac

SEQ ID NO: 155 - RNA, 20 nt, Artificial Sequence
  Synthetic Polynucleotide
cugccugcagcaacuccauc

SEQ ID NO: 156 - RNA, 20 nt, Artificial Sequence
  Synthetic Polynucleotide
ccucagccucugccgcaggg

SEQ ID NO: 157 - RNA, 20 nt, Artificial Sequence
  Synthetic Polynucleotide
guguccggaagucgccugcu

SEQ ID NO: 158 - RNA, 20 nt, Artificial Sequence
  Synthetic Polynucleotide
ugcacguguggcucaagcag

SEQ ID NO: 159 - RNA, 20 nt, Artificial Sequence
  Synthetic Polynucleotide
gacaauaaauaccgaggaau

SEQ ID NO: 160 - RNA, 20 nt, Artificial Sequence
  Synthetic Polynucleotide
gccaugacaaucuccgccag

SEQ ID NO: 161 - RNA, 20 nt, Artificial Sequence
  Synthetic Polynucleotide
gcugucccggagaccauccc

SEQ ID NO: 162 - RNA, 20 nt, Artificial Sequence
  Synthetic Polynucleotide
caugaccagguacagguagu

SEQ ID NO: 163 - RNA, 20 nt, Artificial Sequence
  Synthetic Polynucleotide
agcgcccaccagucacacuc

SEQ ID NO: 164 - RNA, 20 nt, Artificial Sequence
  Synthetic Polynucleotide
ucucagugcauccaaaacgu

SEQ ID NO: 165 - RNA, 20 nt, Artificial Sequence
  Synthetic Polynucleotide
uuugggcagauggagggccu

SEQ ID NO: 166 - RNA, 20 nt, Artificial Sequence
  Synthetic Polynucleotide
gaugucccugugcacguagc

SEQ ID NO: 167 - RNA, 20 nt, Artificial Sequence
  Synthetic Polynucleotide
cagcagugucagcagguccc

SEQ ID NO: 168 - RNA, 20 nt, Artificial Sequence
  Synthetic Polynucleotide
caugacaaucuccgccaggu

SEQ ID NO: 169 - RNA, 20 nt, Artificial Sequence
  Synthetic Polynucleotide
acuuguucaugaucuucaug

SEQ ID NO: 170 - RNA, 20 nt, Artificial Sequence
  Synthetic Polynucleotide
guggaauccgcguagaaggg

SEQ ID NO: 171 - RNA, 20 nt, Artificial Sequence
  Synthetic Polynucleotide
uggccaugacaaucuccgcc

SEQ ID NO: 172 - RNA, 20 nt, Artificial Sequence
  Synthetic Polynucleotide
gggacagacaauaaauaccg

SEQ ID NO: 173 - RNA, 20 nt, Artificial Sequence
  Synthetic Polynucleotide
ccgcuccccaaacuugcuca

SEQ ID NO: 174 - RNA, 20 nt, Artificial Sequence
  Synthetic Polynucleotide
cggcucaggcucugccgggu

SEQ ID NO: 175 - RNA, 20 nt, Artificial Sequence
  Synthetic Polynucleotide
ggcuccugggcggcgccaga

SEQ ID NO: 176 - RNA, 20 nt, Artificial Sequence
  Synthetic Polynucleotide
uuucccgaguaagcaggcag

SEQ ID NO: 177 - RNA, 20 nt, Artificial Sequence
  Synthetic Polynucleotide
ggauguugucggguuugaug

SEQ ID NO: 178 - RNA, 20 nt, Artificial Sequence
  Synthetic Polynucleotide
cagguaguucucauccugga

SEQ ID NO: 179 - RNA, 20 nt, Artificial Sequence
  Synthetic Polynucleotide
ugcccauagaacauuucaua

SEQ ID NO: 180 - RNA, 20 nt, Artificial Sequence
  Synthetic Polynucleotide
uaguucucauccuggaaggc

SEQ ID NO: 181 - RNA, 20 nt, Artificial Sequence
  Synthetic Polynucleotide
augucccugugcacguagcc

SEQ ID NO: 182 - RNA, 20 nt, Artificial Sequence
  Synthetic Polynucleotide
cgggcccggaucacaggacu

SEQ ID NO: 183 - RNA, 20 nt, Artificial Sequence
  Synthetic Polynucleotide
uggacgaucuugccauaggu

SEQ ID NO: 184 - RNA, 20 nt, Artificial Sequence
  Synthetic Polynucleotide
guuggccggcgugggccacc

SEQ ID NO: 185 - RNA, 20 nt, Artificial Sequence
  Synthetic Polynucleotide
cucagugcauccaaaacgug

SEQ ID NO: 186 - RNA, 20 nt, Artificial Sequence
  Synthetic Polynucleotide
ucgaaguugcaugugucggu

SEQ ID NO: 187 - RNA, 20 nt, Artificial Sequence
  Synthetic Polynucleotide
uggaacacggacggcccggc

SEQ ID NO: 188 - RNA, 20 nt, Artificial Sequence
  Synthetic Polynucleotide
ccgagagcagcgcaagugag

SEQ ID NO: 189 - RNA, 20 nt, Artificial Sequence
  Synthetic Polynucleotide
uccugcaacugccggacgug

SEQ ID NO: 190 - RNA, 20 nt, Artificial Sequence
  Synthetic Polynucleotide
ucaccaacacgucccucucc

SEQ ID NO: 191 - RNA, 20 nt, Artificial Sequence
  Synthetic Polynucleotide
ugccugcagcaacuccaucc

SEQ ID NO: 192 - RNA, 20 nt, Artificial Sequence
  Synthetic Polynucleotide
uuggccggcgugggccacca

SEQ ID NO: 193 - RNA, 20 nt, Artificial Sequence
  Synthetic Polynucleotide
gagccucugccucgcguagu

SEQ ID NO: 194 - RNA, 20 nt, Artificial Sequence
  Synthetic Polynucleotide
aagggcgucugcccauagaa

SEQ ID NO: 195 - RNA, 20 nt, Artificial Sequence
  Synthetic Polynucleotide
acagacaauaaauaccgagg

SEQ ID NO: 196 - RNA, 20 nt, Artificial Sequence
  Synthetic Polynucleotide
ggacagacaauaaauaccga

SEQ ID NO: 197 - RNA, 20 nt, Artificial Sequence
  Synthetic Polynucleotide
acgugugccucuaggucccg

SEQ ID NO: 198 - RNA, 20 nt, Artificial Sequence
  Synthetic Polynucleotide
ggcacgagacagaacaacgg

SEQ ID NO: 199 - RNA, 20 nt, Artificial Sequence
  Synthetic Polynucleotide
ugaccagguacagguaguuc

SEQ ID NO: 200 - RNA, 20 nt, Artificial Sequence
  Synthetic Polynucleotide
cucugccgggugagcaccuc

SEQ ID NO: 201 - RNA, 20 nt, Artificial Sequence
  Synthetic Polynucleotide
gacaaucuccgccagguaga

SEQ ID NO: 202 - RNA, 20 nt, Artificial Sequence
  Synthetic Polynucleotide
ucuccgccagguagaagcgc

SEQ ID NO: 203 - RNA, 20 nt, Artificial Sequence
  Synthetic Polynucleotide
cucugccucgcguaguugac

SEQ ID NO: 204 - RNA, 20 nt, Artificial Sequence
  Synthetic Polynucleotide
cuuugggcagauggagggcc

SEQ ID NO: 205 - RNA, 20 nt, Artificial Sequence
  Synthetic Polynucleotide
acagguaguucucauccugg

SEQ ID NO: 206 - RNA, 20 nt, Artificial Sequence
  Synthetic Polynucleotide
ccaaacuugcucagcagugu

SEQ ID NO: 207 - RNA, 20 nt, Artificial Sequence
  Synthetic Polynucleotide
ucggguuugaugucccugug

SEQ ID NO: 208 - RNA, 20 nt, Artificial Sequence
  Synthetic Polynucleotide
ggcuugcugccuucccaggc

SEQ ID NO: 209 - RNA, 20 nt, Artificial Sequence
  Synthetic Polynucleotide
uacagguaguucucauccug

SEQ ID NO: 210 - RNA, 20 nt, Artificial Sequence
  Synthetic Polynucleotide
uugcccauccacgucagggc

SEQ ID NO: 211 - RNA, 20 nt, Artificial Sequence
  Synthetic Polynucleotide
agguacagguaguucucauc

SEQ ID NO: 212 - RNA, 20 nt, Artificial Sequence
  Synthetic Polynucleotide
gacagacaauaaauaccgag

SEQ ID NO: 213 - RNA, 20 nt, Artificial Sequence
  Synthetic Polynucleotide
uagaacauuucauaggcgaa

SEQ ID NO: 214 - RNA, 20 nt, Artificial Sequence
  Synthetic Polynucleotide
agggccuuuuauucgcgagg

SEQ ID NO: 215 - RNA, 20 nt, Artificial Sequence
  Synthetic Polynucleotide
gccucgcguaguugacuggc

SEQ ID NO: 216 - RNA, 20 nt, Artificial Sequence
  Synthetic Polynucleotide
ccagcaggauguugucgggu

SEQ ID NO: 217 - RNA, 20 nt, Artificial Sequence
  Synthetic Polynucleotide
guaguugacuggcgaaguuc

SEQ ID NO: 218 - RNA, 20 nt, Artificial Sequence
  Synthetic Polynucleotide
ugcggauggccuccaucucc

SEQ ID NO: 219 - RNA, 20 nt, Artificial Sequence
  Synthetic Polynucleotide
acaaucuccgccagguagaa

SEQ ID NO: 220 - RNA, 20 nt, Artificial Sequence
  Synthetic Polynucleotide
gcgaauacacccagcgccca

SEQ ID NO: 221 - RNA, 20 nt, Artificial Sequence
  Synthetic Polynucleotide
guaguucucauccuggaagg

SEQ ID NO: 222 - RNA, 20 nt, Artificial Sequence
  Synthetic Polynucleotide
ggcucaggcucugccgggug

SEQ ID NO: 223 - RNA, 20 nt, Artificial Sequence
  Synthetic Polynucleotide
ccauucaccaacacgucccu

SEQ ID NO: 224 - RNA, 20 nt, Artificial Sequence
  Synthetic Polynucleotide
accagguacagguaguucuc

SEQ ID NO: 225 - RNA, 20 nt, Artificial Sequence
  Synthetic Polynucleotide
cugcaguuugcccauccacg

SEQ ID NO: 226 - RNA, 20 nt, Artificial Sequence
  Synthetic Polynucleotide
uuguucaugaucuucauggc

SEQ ID NO: 227 - RNA, 20 nt, Artificial Sequence
  Synthetic Polynucleotide
uuugaugucccugugcacgu

SEQ ID NO: 228 - RNA, 20 nt, Artificial Sequence
  Synthetic Polynucleotide
gcgguccagcaggauguugu

SEQ ID NO: 229 - RNA, 20 nt, Artificial Sequence
  Synthetic Polynucleotide
gucuauggccaugacaaucu

SEQ ID NO: 230 - RNA, 20 nt, Artificial Sequence
  Synthetic Polynucleotide
ggagcagggaaagcgccucc

SEQ ID NO: 231 - RNA, 20 nt, Artificial Sequence
  Synthetic Polynucleotide
ugccucgcguaguugacugg

SEQ ID NO: 232 - RNA, 20 nt, Artificial Sequence
  Synthetic Polynucleotide
gcggauggccuccaucuccc

SEQ ID NO: 233 - RNA, 20 nt, Artificial Sequence
  Synthetic Polynucleotide
uuucauaggcgaauacaccc

SEQ ID NO: 234 - RNA, 20 nt, Artificial Sequence
  Synthetic Polynucleotide
gccugucagcgagucggagg

SEQ ID NO: 235 - RNA, 20 nt, Artificial Sequence
  Synthetic Polynucleotide
ccacuucagcuguuucaucc

SEQ ID NO: 236 - RNA, 20 nt, Artificial Sequence
  Synthetic Polynucleotide
cauccgcuccugcaacugcc

SEQ ID NO: 237 - RNA, 20 nt, Artificial Sequence
  Synthetic Polynucleotide
ucuaggguucagggagcgcg

SEQ ID NO: 238 - RNA, 20 nt, Artificial Sequence
  Synthetic Polynucleotide
caccaacacgucccucuccu

SEQ ID NO: 239 - RNA, 20 nt, Artificial Sequence
  Synthetic Polynucleotide
caggagcagggaaagcgccu

SEQ ID NO: 240 - RNA, 20 nt, Artificial Sequence
  Synthetic Polynucleotide
caaucuccgccagguagaag

SEQ ID NO: 241 - RNA, 20 nt, Artificial Sequence
  Synthetic Polynucleotide
auguugucggguuugauguc

SEQ ID NO: 242 - RNA, 20 nt, Artificial Sequence
  Synthetic Polynucleotide
ccauccgcuccugcaacugc

SEQ ID NO: 243 - RNA, 20 nt, Artificial Sequence
  Synthetic Polynucleotide
gcgucaccucggccucagcc

SEQ ID NO: 244 - RNA, 20 nt, Artificial Sequence
  Synthetic Polynucleotide
gagggccuuuuauucgcgag

SEQ ID NO: 245 - RNA, 20 nt, Artificial Sequence
  Synthetic Polynucleotide
agcggcagagagaggugcuc

SEQ ID NO: 246 - RNA, 20 nt, Artificial Sequence
  Synthetic Polynucleotide
cauccaaaacguggauuggg

SEQ ID NO: 247 - RNA, 20 nt, Artificial Sequence
  Synthetic Polynucleotide
uugggcagauggagggccuu

SEQ ID NO: 248 - RNA, 20 nt, Artificial Sequence
  Synthetic Polynucleotide
ccucugccucgcguaguuga

SEQ ID NO: 249 - RNA, 20 nt, Artificial Sequence
  Synthetic Polynucleotide
acagaacaacggcgaacagg

SEQ ID NO: 250 - RNA, 20 nt, Artificial Sequence
  Synthetic Polynucleotide
caggauguugucggguuuga

SEQ ID NO: 251 - RNA, 20 nt, Artificial Sequence
  Synthetic Polynucleotide
cggccucagccucugccgca

SEQ ID NO: 252 - RNA, 20 nt, Artificial Sequence
  Synthetic Polynucleotide
cagcaggauguugucggguu

SEQ ID NO: 253 - RNA, 20 nt, Artificial Sequence
  Synthetic Polynucleotide
gcagagagaggugcuccuug

SEQ ID NO: 254 - RNA, 20 nt, Artificial Sequence
  Synthetic Polynucleotide
uccaguuccauggguguggg

SEQ ID NO: 255 - RNA, 20 nt, Artificial Sequence
  Synthetic Polynucleotide
ccucagccuggccgaaagaa

SEQ ID NO: 256 - RNA, 20 nt, Artificial Sequence
  Synthetic Polynucleotide
gggccuuuuauucgcgaggg

SEQ ID NO: 257 - RNA, 20 nt, Artificial Sequence
  Synthetic Polynucleotide
gucggccaggcggauguggc

SEQ ID NO: 258 - RNA, 20 nt, Artificial Sequence
  Synthetic Polynucleotide
gcuugcugccuucccaggcc

SEQ ID NO: 259 - RNA, 20 nt, Artificial Sequence
  Synthetic Polynucleotide
gguccagcaggauguugucg

SEQ ID NO: 260 - RNA, 20 nt, Artificial Sequence
  Synthetic Polynucleotide
cggagaccaucccagucgag

SEQ ID NO: 261 - RNA, 20 nt, Artificial Sequence
  Synthetic Polynucleotide
ucugccucgcguaguugacu

SEQ ID NO: 262 - RNA, 20 nt, Artificial Sequence
  Synthetic Polynucleotide
agguaguucucauccuggaa

SEQ ID NO: 263 - RNA, 20 nt, Artificial Sequence
  Synthetic Polynucleotide
uccuuguaguggacgaucuu

SEQ ID NO: 264 - RNA, 20 nt, Artificial Sequence
  Synthetic Polynucleotide
gcauccaaaacguggauugg

SEQ ID NO: 265 - RNA, 20 nt, Artificial Sequence
  Synthetic Polynucleotide
guccagcaggauguugucgg

SEQ ID NO: 266 - RNA, 20 nt, Artificial Sequence
  Synthetic Polynucleotide
agcucccgcagcgucaccuc

SEQ ID NO: 267 - RNA, 20 nt, Artificial Sequence
  Synthetic Polynucleotide
cgagagcagcgcaagugagg

SEQ ID NO: 268 - RNA, 20 nt, Artificial Sequence
  Synthetic Polynucleotide
cagggaaagcgccuccgaua

SEQ ID NO: 269 - RNA, 20 nt, Artificial Sequence
  Synthetic Polynucleotide
auuucauaggcgaauacacc

SEQ ID NO: 270 - RNA, 20 nt, Artificial Sequence
  Synthetic Polynucleotide
ucggccaggcggauguggcc

SEQ ID NO: 271 - RNA, 20 nt, Artificial Sequence
  Synthetic Polynucleotide
aagggauguguccggaaguc

SEQ ID NO: 272 - RNA, 20 nt, Artificial Sequence
  Synthetic Polynucleotide
cuuguaguggacgaucuugc

SEQ ID NO: 273 - RNA, 20 nt, Artificial Sequence
  Synthetic Polynucleotide
agucggccaggcggaugugg

SEQ ID NO: 274 - RNA, 20 nt, Artificial Sequence
  Synthetic Polynucleotide
gccucagccuggccgaaaga

SEQ ID NO: 275 - RNA, 20 nt, Artificial Sequence
  Synthetic Polynucleotide
agcgucaccucggccucagc

SEQ ID NO: 276 - RNA, 20 nt, Artificial Sequence
  Synthetic Polynucleotide
cagcggcagagagaggugcu

SEQ ID NO: 277 - RNA, 20 nt, Artificial Sequence
  Synthetic Polynucleotide
ccagcggcagagagaggugc

SEQ ID NO: 278 - RNA, 20 nt, Artificial Sequence
  Synthetic Polynucleotide
uuguaguggacgaucuugcc

SEQ ID NO: 279 - RNA, 20 nt, Artificial Sequence
  Synthetic Polynucleotide
agggaaagcgccuccgauag

SEQ ID NO: 280 - RNA, 20 nt, Artificial Sequence
  Synthetic Polynucleotide
gggaaagcgccuccgauagg

SEQ ID NO: 281 - DNA, 20 bp, Artificial Sequence
  Synthetic Polynucleotide
ggcagcaagccgggccgtcc

SEQ ID NO: 282 - DNA, 20 bp, Artificial Sequence
  Synthetic Polynucleotide
cagtcctgtgatccgggccc

SEQ ID NO: 283 - DNA, 20 bp, Artificial Sequence
  Synthetic Polynucleotide
gacactgctgagcaagtttg

SEQ ID NO: 284 - DNA, 20 bp, Artificial Sequence
  Synthetic Polynucleotide
tgacactgctgagcaagttt

SEQ ID NO: 285 - DNA, 20 bp, Artificial Sequence
  Synthetic Polynucleotide
cgggagatggaggccatccg

SEQ ID NO: 286 - DNA, 20 bp, Artificial Sequence
  Synthetic Polynucleotide
gggagcggattccggccgag

SEQ ID NO: 287 - DNA, 20 bp, Artificial Sequence
  Synthetic Polynucleotide
ggagcggattccggccgaga

SEQ ID NO: 288 - DNA, 20 bp, Artificial Sequence
  Synthetic Polynucleotide
cctgctgacactgctgagca

SEQ ID NO: 289 - DNA, 20 bp, Artificial Sequence
  Synthetic Polynucleotide
agggacatcaaacccgacaa

SEQ ID NO: 290 - DNA, 20 bp, Artificial Sequence
  Synthetic Polynucleotide
gggacatcaaacccgacaac

SEQ ID NO: 291 - DNA, 20 bp, Artificial Sequence
  Synthetic Polynucleotide
gcgcgcttctacctggcgga

SEQ ID NO: 292 - DNA, 20 bp, Artificial Sequence
  Synthetic Polynucleotide
cgggccaggtgtatgccatg

SEQ ID NO: 293 - DNA, 20 bp, Artificial Sequence
  Synthetic Polynucleotide
ctgacactgctgagcaagtt

SEQ ID NO: 294 - DNA, 20 bp, Artificial Sequence
  Synthetic Polynucleotide
ggcggtggatcacgcagctg

SEQ ID NO: 295 - DNA, 20 bp, Artificial Sequence
  Synthetic Polynucleotide
gagacactgtcggacattcg

SEQ ID NO: 296 - DNA, 20 bp, Artificial Sequence
  Synthetic Polynucleotide
acatccgcctggccgacttc

SEQ ID NO: 297 - DNA, 20 bp, Artificial Sequence
  Synthetic Polynucleotide
cagggacatcaaacccgaca

SEQ ID NO: 298 - DNA, 20 bp, Artificial Sequence
  Synthetic Polynucleotide
ccgggagatggaggccatcc

SEQ ID NO: 299 - DNA, 20 bp, Artificial Sequence
  Synthetic Polynucleotide
atcaaacccgacaacatcct

SEQ ID NO: 300 - DNA, 20 bp, Artificial Sequence
  Synthetic Polynucleotide
acagggacatcaaacccgac

SEQ ID NO: 301 - DNA, 20 bp, Artificial Sequence
  Synthetic Polynucleotide
gtacctggtcatggagtatt

SEQ ID NO: 302 - DNA, 20 bp, Artificial Sequence
  Synthetic Polynucleotide
ccatgaagatcatgaacaag

SEQ ID NO: 303 - DNA, 20 bp, Artificial Sequence
  Synthetic Polynucleotide
ccacgttttggatgcactga

SEQ ID NO: 304 - DNA, 20 bp, Artificial Sequence
  Synthetic Polynucleotide
tgggatggtctccgggacag

SEQ ID NO: 305 - DNA, 20 bp, Artificial Sequence
  Synthetic Polynucleotide
gacagtgaggtcccaggccc

SEQ ID NO: 306 - DNA, 20 bp, Artificial Sequence
  Synthetic Polynucleotide
gtcatggagtattacgtggg

SEQ ID NO: 307 - DNA, 20 bp, Artificial Sequence
  Synthetic Polynucleotide
cggctgtccctgcggcagag

SEQ ID NO: 308 - DNA, 20 bp, Artificial Sequence
  Synthetic Polynucleotide
cggcttggctacgtgcacag

SEQ ID NO: 309 - DNA, 20 bp, Artificial Sequence
  Synthetic Polynucleotide
ggccctgacgtggatgggca

SEQ ID NO: 310 - DNA, 20 bp, Artificial Sequence
  Synthetic Polynucleotide
cctggcctatcggaggcgct

SEQ ID NO: 311 - DNA, 20 bp, Artificial Sequence
  Synthetic Polynucleotide
ccggcttggctacgtgcaca

SEQ ID NO: 312 - DNA, 20 bp, Artificial Sequence
  Synthetic Polynucleotide
agaactacctgtacctggtc

SEQ ID NO: 313 - DNA, 20 bp, Artificial Sequence
  Synthetic Polynucleotide
gcggattccggccgagatgg

SEQ ID NO: 314 - DNA, 20 bp, Artificial Sequence
  Synthetic Polynucleotide
agcggattccggccgagatg

SEQ ID NO: 315 - DNA, 20 bp, Artificial Sequence
  Synthetic Polynucleotide
acggcggagacctatggcaa

SEQ ID NO: 316 - DNA, 20 bp, Artificial Sequence
  Synthetic Polynucleotide
acatcctgctggaccgctgt

SEQ ID NO: 317 - DNA, 20 bp, Artificial Sequence
  Synthetic Polynucleotide
tggcctatcggaggcgcttt

SEQ ID NO: 318 - DNA, 20 bp, Artificial Sequence
  Synthetic Polynucleotide
cacatcccttcttctttggc

SEQ ID NO: 319 - DNA, 20 bp, Artificial Sequence
  Synthetic Polynucleotide
tggtcatggagtattacgtg

SEQ ID NO: 320 - DNA, 20 bp, Artificial Sequence
  Synthetic Polynucleotide
gagcggattccggccgagat

SEQ ID NO: 321 - DNA, 20 bp, Artificial Sequence
  Synthetic Polynucleotide
agcggtagtgaagatgaagc

SEQ ID NO: 322 - DNA, 20 bp, Artificial Sequence
  Synthetic Polynucleotide
cggattccggccgagatggc

SEQ ID NO: 323 - DNA, 20 bp, Artificial Sequence
  Synthetic Polynucleotide
agttccagcggctgtccctg

SEQ ID NO: 324 - DNA, 20 bp, Artificial Sequence
  Synthetic Polynucleotide
tacctggcggagattgtcat

SEQ ID NO: 325 - DNA, 20 bp, Artificial Sequence
  Synthetic Polynucleotide
tggcggagattgtcatggcc

SEQ ID NO: 326 - DNA, 20 bp, Artificial Sequence
  Synthetic Polynucleotide
tgtacctggtcatggagtat

SEQ ID NO: 327 - DNA, 20 bp, Artificial Sequence
  Synthetic Polynucleotide
caactacgcgaggcagaggc

SEQ ID NO: 328 - DNA, 20 bp, Artificial Sequence
  Synthetic Polynucleotide
ggagacactgtcggacattc

SEQ ID NO: 329 - DNA, 20 bp, Artificial Sequence
  Synthetic Polynucleotide
agctgcgggcagatggaacg

SEQ ID NO: 330 - DNA, 20 bp, Artificial Sequence
  Synthetic Polynucleotide
caagatcgtccactacaagg

SEQ ID NO: 331 - DNA, 20 bp, Artificial Sequence
  Synthetic Polynucleotide
cgcttctacctggcggagat

SEQ ID NO: 332 - DNA, 20 bp, Artificial Sequence
  Synthetic Polynucleotide
ctcacccggcagagcctgag

SEQ ID NO: 333 - DNA, 20 bp, Artificial Sequence
  Synthetic Polynucleotide
gcggtagtgaagatgaagca

SEQ ID NO: 334 - DNA, 20 bp, Artificial Sequence
  Synthetic Polynucleotide
caaacccgacaacatcctgc

SEQ ID NO: 335 - DNA, 20 bp, Artificial Sequence
  Synthetic Polynucleotide
ctgcggcagaggctgaggcc

SEQ ID NO: 336 - DNA, 20 bp, Artificial Sequence
  Synthetic Polynucleotide
ggacatcaaacccgacaaca

SEQ ID NO: 337 - DNA, 20 bp, Artificial Sequence
  Synthetic Polynucleotide
ggtcatggagtattacgtgg

SEQ ID NO: 338 - DNA, 20 bp, Artificial Sequence
  Synthetic Polynucleotide
gctgcgggcagatggaacgg

SEQ ID NO: 339 - DNA, 20 bp, Artificial Sequence
  Synthetic Polynucleotide
tctgcctgcttactcgggaa

SEQ ID NO: 340 - DNA, 20 bp, Artificial Sequence
  Synthetic Polynucleotide
ggtgtatgccatgaagatca

SEQ ID NO: 341 - DNA, 20 bp, Artificial Sequence
  Synthetic Polynucleotide
cagttccagcggctgtccct

SEQ ID NO: 342 - DNA, 20 bp, Artificial Sequence
  Synthetic Polynucleotide
tgcacagggacatcaaaccc

SEQ ID NO: 343 - DNA, 20 bp, Artificial Sequence
  Synthetic Polynucleotide
ctacctggcggagattgtca

SEQ ID NO: 344 - DNA, 20 bp, Artificial Sequence
  Synthetic Polynucleotide
catcctgctggaccgctgtg

SEQ ID NO: 345 - DNA, 20 bp, Artificial Sequence
  Synthetic Polynucleotide
gggcagacgcccttctacgc

SEQ ID NO: 346 - DNA, 20 bp, Artificial Sequence
  Synthetic Polynucleotide
tccctgcggcagaggctgag

SEQ ID NO: 347 - DNA, 20 bp, Artificial Sequence
  Synthetic Polynucleotide
cgttttggatgcactgagac

SEQ ID NO: 348 - DNA, 20 bp, Artificial Sequence
  Synthetic Polynucleotide
gacctatggcaagatcgtcc

SEQ ID NO: 349 - DNA, 20 bp, Artificial Sequence
  Synthetic Polynucleotide
ggacctgctgacactgctga

SEQ ID NO: 350 - DNA, 20 bp, Artificial Sequence
  Synthetic Polynucleotide
gtctggcgccgcccaggagc

SEQ ID NO: 351 - DNA, 20 bp, Artificial Sequence
  Synthetic Polynucleotide
aaacccgacaacatcctgct

SEQ ID NO: 352 - DNA, 20 bp, Artificial Sequence
  Synthetic Polynucleotide
cggcagttgcaggagcggat

SEQ ID NO: 353 - DNA, 20 bp, Artificial Sequence
  Synthetic Polynucleotide
gaggcgctttccctgctcct

SEQ ID NO: 354 - DNA, 20 bp, Artificial Sequence
  Synthetic Polynucleotide
gaagcagacgggccaggtgt

SEQ ID NO: 355 - DNA, 20 bp, Artificial Sequence
  Synthetic Polynucleotide
tgtgactggtgggcgctggg

SEQ ID NO: 356 - DNA, 20 bp, Artificial Sequence
  Synthetic Polynucleotide
ttgctgcaggcagagggagc

SEQ ID NO: 357 - DNA, 20 bp, Artificial Sequence
  Synthetic Polynucleotide
tcacccggcagagcctgagc

SEQ ID NO: 358 - DNA, 20 bp, Artificial Sequence
  Synthetic Polynucleotide
tacgtgcacagggacatcaa

SEQ ID NO: 359 - DNA, 20 bp, Artificial Sequence
  Synthetic Polynucleotide
cctgcggcagaggctgaggc

SEQ ID NO: 360 - DNA, 20 bp, Artificial Sequence
  Synthetic Polynucleotide
cttccaggatgagaactacc

SEQ ID NO: 361 - DNA, 20 bp, Artificial Sequence
  Synthetic Polynucleotide
agtgtgactggtgggcgctg

SEQ ID NO: 362 - DNA, 20 bp, Artificial Sequence
  Synthetic Polynucleotide
cactgctgagcaagtttggg

SEQ ID NO: 363 - DNA, 20 bp, Artificial Sequence
  Synthetic Polynucleotide
cggcggagacctatggcaag

SEQ ID NO: 364 - DNA, 20 bp, Artificial Sequence
  Synthetic Polynucleotide
aagcagacgggccaggtgta

SEQ ID NO: 365 - DNA, 20 bp, Artificial Sequence
  Synthetic Polynucleotide
gtgtgactggtgggcgctgg

SEQ ID NO: 366 - DNA, 20 bp, Artificial Sequence
  Synthetic Polynucleotide
ctttcggccaggctgaggcc

SEQ ID NO: 367 - DNA, 20 bp, Artificial Sequence
  Synthetic Polynucleotide
gcttctacctggcggagatt

SEQ ID NO: 368 - DNA, 20 bp, Artificial Sequence
  Synthetic Polynucleotide
acgggccaggtgtatgccat

SEQ ID NO: 369 - DNA, 20 bp, Artificial Sequence
  Synthetic Polynucleotide
cctggcggagattgtcatgg

SEQ ID NO: 370 - DNA, 20 bp, Artificial Sequence
  Synthetic Polynucleotide
ctgctgagcaagtttgggga

SEQ ID NO: 371 - DNA, 20 bp, Artificial Sequence
  Synthetic Polynucleotide
acatcaaacccgacaacatc

SEQ ID NO: 372 - DNA, 20 bp, Artificial Sequence
  Synthetic Polynucleotide
cctgacgtggatgggcaaac

SEQ ID NO: 373 - DNA, 20 bp, Artificial Sequence
  Synthetic Polynucleotide
gcagcaagccgggccgtccg

SEQ ID NO: 374 - DNA, 20 bp, Artificial Sequence
  Synthetic Polynucleotide
cgcgcttctacctggcggag

SEQ ID NO: 375 - DNA, 20 bp, Artificial Sequence
  Synthetic Polynucleotide
aggatgagaactacctgtac

SEQ ID NO: 376 - DNA, 20 bp, Artificial Sequence
  Synthetic Polynucleotide
gttctatgggcagacgccct

SEQ ID NO: 377 - DNA, 20 bp, Artificial Sequence
  Synthetic Polynucleotide
ctgctggaccgctgtggcca

SEQ ID NO: 378 - DNA, 20 bp, Artificial Sequence
  Synthetic Polynucleotide
aacttcgccagtcaactacg

SEQ ID NO: 379 - DNA, 20 bp, Artificial Sequence
  Synthetic Polynucleotide
gcggctgtccctgcggcaga

SEQ ID NO: 380 - DNA, 20 bp, Artificial Sequence
  Synthetic Polynucleotide
ctggcctatcggaggcgctt

SEQ ID NO: 381 - DNA, 20 bp, Artificial Sequence
  Synthetic Polynucleotide
ctgttcgccgttgttctgtc

SEQ ID NO: 382 - DNA, 20 bp, Artificial Sequence
  Synthetic Polynucleotide
acctgctgacactgctgagc

SEQ ID NO: 383 - DNA, 20 bp, Artificial Sequence
  Synthetic Polynucleotide
gtgtatgccatgaagatcat

SEQ ID NO: 384 - DNA, 20 bp, Artificial Sequence
  Synthetic Polynucleotide
ccctgacgtggatgggcaaa

SEQ ID NO: 385 - DNA, 20 bp, Artificial Sequence
  Synthetic Polynucleotide
gctgacactgctgagcaagt

SEQ ID NO: 386 - DNA, 20 bp, Artificial Sequence
  Synthetic Polynucleotide
ctacgtgcacagggacatca

SEQ ID NO: 387 - DNA, 20 bp, Artificial Sequence
  Synthetic Polynucleotide
cccgacattcctcggtattt

SEQ ID NO: 388 - DNA, 20 bp, Artificial Sequence
  Synthetic Polynucleotide
gggcgctgggtgtattcgcc

SEQ ID NO: 389 - DNA, 20 bp, Artificial Sequence
  Synthetic Polynucleotide
ttcctcggtatttattgtct

SEQ ID NO: 390 - DNA, 20 bp, Artificial Sequence
  Synthetic Polynucleotide
gtgaagatgaagcagacggg

SEQ ID NO: 391 - DNA, 20 bp, Artificial Sequence
  Synthetic Polynucleotide
gatggagttgctgcaggcag

SEQ ID NO: 392 - DNA, 20 bp, Artificial Sequence
  Synthetic Polynucleotide
ccctgcggcagaggctgagg

SEQ ID NO: 393 - DNA, 20 bp, Artificial Sequence
  Synthetic Polynucleotide
agcaggcgacttccggacac

SEQ ID NO: 394 - DNA, 20 bp, Artificial Sequence
  Synthetic Polynucleotide
ctgcttgagccacacgtgca

SEQ ID NO: 395 - DNA, 20 bp, Artificial Sequence
  Synthetic Polynucleotide
attcctcggtatttattgtc

SEQ ID NO: 396 - DNA, 20 bp, Artificial Sequence
  Synthetic Polynucleotide
ctggcggagattgtcatggc

SEQ ID NO: 397 - DNA, 20 bp, Artificial Sequence
  Synthetic Polynucleotide
gggatggtctccgggacagc

SEQ ID NO: 398 - DNA, 20 bp, Artificial Sequence
  Synthetic Polynucleotide
actacctgtacctggtcatg

SEQ ID NO: 399 - DNA, 20 bp, Artificial Sequence
  Synthetic Polynucleotide
gagtgtgactggtgggcgct

SEQ ID NO: 400 - DNA, 20 bp, Artificial Sequence
  Synthetic Polynucleotide
acgttttggatgcactgaga

SEQ ID NO: 401 - DNA, 20 bp, Artificial Sequence
  Synthetic Polynucleotide
aggccctccatctgcccaaa

SEQ ID NO: 402 - DNA, 20 bp, Artificial Sequence
  Synthetic Polynucleotide
gctacgtgcacagggacatc

SEQ ID NO: 403 - DNA, 20 bp, Artificial Sequence
  Synthetic Polynucleotide
gggacctgctgacactgctg

SEQ ID NO: 404 - DNA, 20 bp, Artificial Sequence
  Synthetic Polynucleotide
acctggcggagattgtcatg

SEQ ID NO: 405 - DNA, 20 bp, Artificial Sequence
  Synthetic Polynucleotide
catgaagatcatgaacaagt

SEQ ID NO: 406 - DNA, 20 bp, Artificial Sequence
  Synthetic Polynucleotide
cccttctacgcggattccac

SEQ ID NO: 407 - DNA, 20 bp, Artificial Sequence
  Synthetic Polynucleotide
ggcggagattgtcatggcca

SEQ ID NO: 408 - DNA, 20 bp, Artificial Sequence
  Synthetic Polynucleotide
cggtatttattgtctgtccc

SEQ ID NO: 409 - DNA, 20 bp, Artificial Sequence
  Synthetic Polynucleotide
tgagcaagtttggggagcgg

SEQ ID NO: 410 - DNA, 20 bp, Artificial Sequence
  Synthetic Polynucleotide
acccggcagagcctgagccg

SEQ ID NO: 411 - DNA, 20 bp, Artificial Sequence
  Synthetic Polynucleotide
tctggcgccgcccaggagcc

SEQ ID NO: 412 - DNA, 20 bp, Artificial Sequence
  Synthetic Polynucleotide
ctgcctgcttactcgggaaa

SEQ ID NO: 413 - DNA, 20 bp, Artificial Sequence
  Synthetic Polynucleotide
catcaaacccgacaacatcc

SEQ ID NO: 414 - DNA, 20 bp, Artificial Sequence
  Synthetic Polynucleotide
tccaggatgagaactacctg

SEQ ID NO: 415 - DNA, 20 bp, Artificial Sequence
  Synthetic Polynucleotide
tatgaaatgttctatgggca

SEQ ID NO: 416 - DNA, 20 bp, Artificial Sequence
  Synthetic Polynucleotide
gccttccaggatgagaacta

SEQ ID NO: 417 - DNA, 20 bp, Artificial Sequence
  Synthetic Polynucleotide
ggctacgtgcacagggacat

SEQ ID NO: 418 - DNA, 20 bp, Artificial Sequence
  Synthetic Polynucleotide
agtcctgtgatccgggcccg

SEQ ID NO: 419 - DNA, 20 bp, Artificial Sequence
  Synthetic Polynucleotide
acctatggcaagatcgtcca

SEQ ID NO: 420 - DNA, 20 bp, Artificial Sequence
  Synthetic Polynucleotide
ggtggcccacgccggccaac

SEQ ID NO: 421 - DNA, 20 bp, Artificial Sequence
  Synthetic Polynucleotide
cacgttttggatgcactgag

SEQ ID NO: 422 - DNA, 20 bp, Artificial Sequence
  Synthetic Polynucleotide
accgacacatgcaacttcga

SEQ ID NO: 423 - DNA, 20 bp, Artificial Sequence
  Synthetic Polynucleotide
gccgggccgtccgtgttcca

SEQ ID NO: 424 - DNA, 20 bp, Artificial Sequence
  Synthetic Polynucleotide
ctcacttgcgctgctctcgg

SEQ ID NO: 425 - DNA, 20 bp, Artificial Sequence
  Synthetic Polynucleotide
cacgtccggcagttgcagga

SEQ ID NO: 426 - DNA, 20 bp, Artificial Sequence
  Synthetic Polynucleotide
ggagagggacgtgttggtga

SEQ ID NO: 427 - DNA, 20 bp, Artificial Sequence
  Synthetic Polynucleotide
ggatggagttgctgcaggca

SEQ ID NO: 428 - DNA, 20 bp, Artificial Sequence
  Synthetic Polynucleotide
tggtggcccacgccggccaa

SEQ ID NO: 429 - DNA, 20 bp, Artificial Sequence
  Synthetic Polynucleotide
actacgcgaggcagaggctc

SEQ ID NO: 430 - DNA, 20 bp, Artificial Sequence
  Synthetic Polynucleotide
ttctatgggcagacgccctt

SEQ ID NO: 431 - DNA, 20 bp, Artificial Sequence
  Synthetic Polynucleotide
cctcggtatttattgtctgt

SEQ ID NO: 432 - DNA, 20 bp, Artificial Sequence
  Synthetic Polynucleotide
tcggtatttattgtctgtcc

SEQ ID NO: 433 - DNA, 20 bp, Artificial Sequence
  Synthetic Polynucleotide
cgggacctagaggcacacgt

SEQ ID NO: 434 - DNA, 20 bp, Artificial Sequence
  Synthetic Polynucleotide
ccgttgttctgtctcgtgcc

SEQ ID NO: 435 - DNA, 20 bp, Artificial Sequence
  Synthetic Polynucleotide
gaactacctgtacctggtca

SEQ ID NO: 436 - DNA, 20 bp, Artificial Sequence
  Synthetic Polynucleotide
gaggtgctcacccggcagag

SEQ ID NO: 437 - DNA, 20 bp, Artificial Sequence
  Synthetic Polynucleotide
tctacctggcggagattgtc

SEQ ID NO: 438 - DNA, 20 bp, Artificial Sequence
  Synthetic Polynucleotide
gcgcttctacctggcggaga

SEQ ID NO: 439 - DNA, 20 bp, Artificial Sequence
  Synthetic Polynucleotide
gtcaactacgcgaggcagag

SEQ ID NO: 440 - DNA, 20 bp, Artificial Sequence
  Synthetic Polynucleotide
ggccctccatctgcccaaag

SEQ ID NO: 441 - DNA, 20 bp, Artificial Sequence
  Synthetic Polynucleotide
ccaggatgagaactacctgt

SEQ ID NO: 442 - DNA, 20 bp, Artificial Sequence
  Synthetic Polynucleotide
acactgctgagcaagtttgg

SEQ ID NO: 443 - DNA, 20 bp, Artificial Sequence
  Synthetic Polynucleotide
cacagggacatcaaacccga

SEQ ID NO: 444 - DNA, 20 bp, Artificial Sequence
  Synthetic Polynucleotide
gcctgggaaggcagcaagcc

SEQ ID NO: 445 - DNA, 20 bp, Artificial Sequence
  Synthetic Polynucleotide
caggatgagaactacctgta

SEQ ID NO: 446 - DNA, 20 bp, Artificial Sequence
  Synthetic Polynucleotide
gccctgacgtggatgggcaa

SEQ ID NO: 447 - DNA, 20 bp, Artificial Sequence
  Synthetic Polynucleotide
gatgagaactacctgtacct

SEQ ID NO: 448 - DNA, 20 bp, Artificial Sequence
  Synthetic Polynucleotide
ctcggtatttattgtctgtc

SEQ ID NO: 449 - DNA, 20 bp, Artificial Sequence
  Synthetic Polynucleotide
ttcgcctatgaaatgttcta

SEQ ID NO: 450 - DNA, 20 bp, Artificial Sequence
  Synthetic Polynucleotide
cctcgcgaataaaaggccct

SEQ ID NO: 451 - DNA, 20 bp, Artificial Sequence
  Synthetic Polynucleotide
gccagtcaactacgcgaggc

SEQ ID NO: 452 - DNA, 20 bp, Artificial Sequence
  Synthetic Polynucleotide
acccgacaacatcctgctgg

SEQ ID NO: 453 - DNA, 20 bp, Artificial Sequence
  Synthetic Polynucleotide
gaacttcgccagtcaactac

SEQ ID NO: 454 - DNA, 20 bp, Artificial Sequence
  Synthetic Polynucleotide
ggagatggaggccatccgca

SEQ ID NO: 455 - DNA, 20 bp, Artificial Sequence
  Synthetic Polynucleotide
ttctacctggcggagattgt

SEQ ID NO: 456 - DNA, 20 bp, Artificial Sequence
  Synthetic Polynucleotide
tgggcgctgggtgtattcgc

SEQ ID NO: 457 - DNA, 20 bp, Artificial Sequence
  Synthetic Polynucleotide
ccttccaggatgagaactac

SEQ ID NO: 458 - DNA, 20 bp, Artificial Sequence
  Synthetic Polynucleotide
cacccggcagagcctgagcc

SEQ ID NO: 459 - DNA, 20 bp, Artificial Sequence
  Synthetic Polynucleotide
agggacgtgttggtgaatgg

SEQ ID NO: 460 - DNA, 20 bp, Artificial Sequence
  Synthetic Polynucleotide
gagaactacctgtacctggt

SEQ ID NO: 461 - DNA, 20 bp, Artificial Sequence
  Synthetic Polynucleotide
cgtggatgggcaaactgcag

SEQ ID NO: 462 - DNA, 20 bp, Artificial Sequence
  Synthetic Polynucleotide
gccatgaagatcatgaacaa

SEQ ID NO: 463 - DNA, 20 bp, Artificial Sequence
  Synthetic Polynucleotide
acgtgcacagggacatcaaa

SEQ ID NO: 464 - DNA, 20 bp, Artificial Sequence
  Synthetic Polynucleotide
acaacatcctgctggaccgc

SEQ ID NO: 465 - DNA, 20 bp, Artificial Sequence
  Synthetic Polynucleotide
agattgtcatggccatagac

SEQ ID NO: 466 - DNA, 20 bp, Artificial Sequence
  Synthetic Polynucleotide
ggaggcgctttccctgctcc

SEQ ID NO: 467 - DNA, 20 bp, Artificial Sequence
  Synthetic Polynucleotide
ccagtcaactacgcgaggca

SEQ ID NO: 468 - DNA, 20 bp, Artificial Sequence
  Synthetic Polynucleotide
gggagatggaggccatccgc

SEQ ID NO: 469 - DNA, 20 bp, Artificial Sequence
  Synthetic Polynucleotide
gggtgtattcgcctatgaaa

SEQ ID NO: 470 - DNA, 20 bp, Artificial Sequence
  Synthetic Polynucleotide
cctccgactcgctgacaggc

SEQ ID NO: 471 - DNA, 20 bp, Artificial Sequence
  Synthetic Polynucleotide
ggatgaaacagctgaagtgg

SEQ ID NO: 472 - DNA, 20 bp, Artificial Sequence
  Synthetic Polynucleotide
ggcagttgcaggagcggatg

SEQ ID NO: 473 - DNA, 20 bp, Artificial Sequence
  Synthetic Polynucleotide
cgcgctccctgaaccctaga

SEQ ID NO: 474 - DNA, 20 bp, Artificial Sequence
  Synthetic Polynucleotide
aggagagggacgtgttggtg

SEQ ID NO: 475 - DNA, 20 bp, Artificial Sequence
  Synthetic Polynucleotide
aggcgctttccctgctcctg

SEQ ID NO: 476 - DNA, 20 bp, Artificial Sequence
  Synthetic Polynucleotide
cttctacctggcggagattg

SEQ ID NO: 477 - DNA, 20 bp, Artificial Sequence
  Synthetic Polynucleotide
gacatcaaacccgacaacat

SEQ ID NO: 478 - DNA, 20 bp, Artificial Sequence
  Synthetic Polynucleotide
gcagttgcaggagcggatgg

SEQ ID NO: 479 - DNA, 20 bp, Artificial Sequence
  Synthetic Polynucleotide
ggctgaggccgaggtgacgc

SEQ ID NO: 480 - DNA, 20 bp, Artificial Sequence
  Synthetic Polynucleotide
ctcgcgaataaaaggccctc

SEQ ID NO: 481 - DNA, 20 bp, Artificial Sequence
  Synthetic Polynucleotide
gagcacctctctctgccgct

SEQ ID NO: 482 - DNA, 20 bp, Artificial Sequence
  Synthetic Polynucleotide
cccaatccacgttttggatg

SEQ ID NO: 483 - DNA, 20 bp, Artificial Sequence
  Synthetic Polynucleotide
aaggccctccatctgcccaa

SEQ ID NO: 484 - DNA, 20 bp, Artificial Sequence
  Synthetic Polynucleotide
tcaactacgcgaggcagagg

SEQ ID NO: 485 - DNA, 20 bp, Artificial Sequence
  Synthetic Polynucleotide
cctgttcgccgttgttctgt

SEQ ID NO: 486 - DNA, 20 bp, Artificial Sequence
  Synthetic Polynucleotide
tcaaacccgacaacatcctg

SEQ ID NO: 487 - DNA, 20 bp, Artificial Sequence
  Synthetic Polynucleotide
tgcggcagaggctgaggccg

SEQ ID NO: 488 - DNA, 20 bp, Artificial Sequence
  Synthetic Polynucleotide
aacccgacaacatcctgctg

SEQ ID NO: 489 - DNA, 20 bp, Artificial Sequence
  Synthetic Polynucleotide
caaggagcacctctctctgc

SEQ ID NO: 490 - DNA, 20 bp, Artificial Sequence
  Synthetic Polynucleotide
cccacacccatggaactgga

SEQ ID NO: 491 - DNA, 20 bp, Artificial Sequence
  Synthetic Polynucleotide
ttctttcggccaggctgagg

SEQ ID NO: 492 - DNA, 20 bp, Artificial Sequence
  Synthetic Polynucleotide
ccctcgcgaataaaaggccc

SEQ ID NO: 493 - DNA, 20 bp, Artificial Sequence
  Synthetic Polynucleotide
gccacatccgcctggccgac

SEQ ID NO: 494 - DNA, 20 bp, Artificial Sequence
  Synthetic Polynucleotide
ggcctgggaaggcagcaagc

SEQ ID NO: 495 - DNA, 20 bp, Artificial Sequence
  Synthetic Polynucleotide
cgacaacatcctgctggacc

SEQ ID NO: 496 - DNA, 20 bp, Artificial Sequence
  Synthetic Polynucleotide
ctcgactgggatggtctccg

SEQ ID NO: 497 - DNA, 20 bp, Artificial Sequence
  Synthetic Polynucleotide
agtcaactacgcgaggcaga

SEQ ID NO: 498 - DNA, 20 bp, Artificial Sequence
  Synthetic Polynucleotide
ttccaggatgagaactacct

SEQ ID NO: 499 - DNA, 20 bp, Artificial Sequence
  Synthetic Polynucleotide
aagatcgtccactacaagga

SEQ ID NO: 500 - DNA, 20 bp, Artificial Sequence
  Synthetic Polynucleotide
ccaatccacgttttggatgc

SEQ ID NO: 501 - DNA, 20 bp, Artificial Sequence
  Synthetic Polynucleotide
ccgacaacatcctgctggac

SEQ ID NO: 502 - DNA, 20 bp, Artificial Sequence
  Synthetic Polynucleotide
gaggtgacgctgcgggagct

SEQ ID NO: 503 - DNA, 20 bp, Artificial Sequence
  Synthetic Polynucleotide
cctcacttgcgctgctctcg

SEQ ID NO: 504 - DNA, 20 bp, Artificial Sequence
  Synthetic Polynucleotide
tatcggaggcgctttccctg

SEQ ID NO: 505 - DNA, 20 bp, Artificial Sequence
  Synthetic Polynucleotide
ggtgtattcgcctatgaaat

SEQ ID NO: 506 - DNA, 20 bp, Artificial Sequence
  Synthetic Polynucleotide
ggccacatccgcctggccga

SEQ ID NO: 507 - DNA, 20 bp, Artificial Sequence
  Synthetic Polynucleotide
gacttccggacacatccctt

SEQ ID NO: 508 - DNA, 20 bp, Artificial Sequence
  Synthetic Polynucleotide
gcaagatcgtccactacaag

SEQ ID NO: 509 - DNA, 20 bp, Artificial Sequence
  Synthetic Polynucleotide
ccacatccgcctggccgact

SEQ ID NO: 510 - DNA, 20 bp, Artificial Sequence
  Synthetic Polynucleotide
tctttcggccaggctgaggc

SEQ ID NO: 511 - DNA, 20 bp, Artificial Sequence
  Synthetic Polynucleotide
gctgaggccgaggtgacgct

SEQ ID NO: 512 - DNA, 20 bp, Artificial Sequence
  Synthetic Polynucleotide
agcacctctctctgccgctg

SEQ ID NO: 513 - DNA, 20 bp, Artificial Sequence
  Synthetic Polynucleotide
gcacctctctctgccgctgg

SEQ ID NO: 514 - DNA, 20 bp, Artificial Sequence
  Synthetic Polynucleotide
ggcaagatcgtccactacaa

SEQ ID NO: 515 - DNA, 20 bp, Artificial Sequence
  Synthetic Polynucleotide
ctatcggaggcgctttccct

SEQ ID NO: 516 - DNA, 20 bp, Artificial Sequence
  Synthetic Polynucleotide
cctatcggaggcgctttccc